(19) United States
(12) Patent Application Publication
(10) Pub. No.: US 2023/0241128 A1
(43) Pub. Date: Aug. 3, 2023

(54) METHODS FOR DIAGNOSING AND TREATING METABOLIC DISEASES

(71) Applicant: Microbiota I – Center (Magic) Limited, Shatin, N.T. (CN)

(72) Inventors: Siew Chien NG, Hong Kong, SAR (CN); Ka Leung Francis Chan, Tai Po, New (CN); Tao Zuo, Qingzhou (CN)

(21) Appl. No.: 17/922,960

(22) PCT Filed: May 4, 2021
(86) PCT No.: PCT/CN2021/091804

§ 371 Date: Nov. 2, 2022

Related U.S. Application Data
(60) Provisional application No. 63/020,181, filed on May 5, 2020.
(60) Provisional application No. 63/091,645, filed on Oct. 14, 2020.

(51) Int. Cl. A61K 35/74; A61P 3/04; A61P 3/10; G01N 33/50

(52) U.S. Cl. CPC A61K 35/74; A61P 3/04; A61P 3/10; G01N 33/5091

(57) ABSTRACT

Methods are provided for treating metabolic diseases by way of modulating recipients' gastrointestinal tract microorganism profile such as by fecal microbiota transplantation (FMT) treatment. Also provided are methods for assessing a patient's risk of developing obesity and/or related metabolic diseases. Further provided are kits and compositions for use in these methods.

### RELATED APPLICATIONS

This application claims priority to U.S. Provisional Patent Application No. 63/020,181, filed May 5, 2020, and U.S. Provisional Patent Application No. 63/091,645, filed Oct. 14, 2020, the contents of each of the above are hereby incorporated by reference in the entirety for all purposes.

### BACKGROUND OF THE INVENTION

As living standards continue to improve globally, the number of individuals who are overweight or even obese is also rapidly increasing. Because of the serious health risks directly associated with excess body weight, this trend of an ever increasing proportion of the general population being overweight has led to a notably higher incidence of many diseases including various metabolic diseases especially diabetes, heart disease, hypertension, and stroke. Obesity and type 2 diabetes mellitus (T2DM) are global public health challenges. For example, in the United States the percentage of obese individuals in the general population has recently exceeded 40%, and the World Health Organization (WHO) estimates that by 2030 the number of people living with diabetes will exceed 350 million worldwide.

Due to the rising incidence of obesity-related diseases especially metabolic diseases, their serious health implications, as well as their profound economic consequences, there exists an urgent need for new and effective means to treat individuals who are either already overweight or obese or at risk of becoming overweight or obese in order to help them reduce their bodyweight to, or maintain their bodyweight at, a lower and more healthful level, thus achieve or maintain normal blood glucose level, cholesterol level (including low-density lipoprotein cholesterol (LDL-C) and high-density lipoprotein cholesterol (HDL-C) levels), and triglyceride level, and ultimately reduce or eliminate their risk of later suffering from serious illnesses such as diabetes and cardiovascular disease. Controlling obesity is thus of critical importance as it is associated with increased risk of comorbidities and complications including T2DM, cerebrovascular incidents, and coronary artery diseases. The present invention fulfills this and other related needs by providing new methods for assessing a patient's risk of developing obesity and related metabolic disease as well as new methods and compositions that can effectively regulate a patient's bodyweight and/or treating related metabolic diseases including T2DM.

### BRIEF SUMMARY OF THE INVENTION

The invention relates to novel methods and compositions useful for treating a metabolic disease such as diabetes as well as for assessing a patient's likelihood of developing a metabolic disease. In particular, the present inventors have discovered that certain microorganism species, especially certain virus and bacteria species, are present at distinctly altered levels in the gastrointestinal (GI) tract of individuals depending on whether or not they have or are at heightened risk of developing a metabolic disease. Health benefits associated with bodyweight reduction such as improved blood glucose, triglyceride, and/or cholesterol level(s) and therefore reduced risks of serious medical conditions such as heart disease, hypertension, stroke, and diabetes can be achieved by modulating the level of pertinent microorganisms in patients' gut, for example, by fecal microbiota transplantation (FMT) treatment or oral administration of beneficial viral and/or bacterial species. These findings also provide new methods for treating a metabolic disease. Thus, in the first aspect, the present invention provides a method for reducing the risk of a metabolic disease or treating a metabolic disease in a subject, comprising administering to the subject a composition comprising an effective amount of one or more of the microbial species selected from the group consisting of Diachasmimorpha longicaudata entomopoxvirus, Megavirus, Oenococcus phage, Saudi moumouvirus, Clostridium botulinum C phage, Emiliania huxleyi virus, and Lausannevirus. In some embodiments, the composition further comprises one or more of the microbial species selected from the group consisting of Gokushovirus, Bacillus phage, Escherichia phage, Streptococcus phage, and Microvirus. In some embodiments, the composition further comprises Candida dubliniensis. In some embodiments, the composition comprises a low abundance of crAssphage or does not contain any crAssphage. In some embodiments, the metabolic disease is obesity, type-1 diabetes, or type-2 diabetes. In some embodiments, the method increases high-density lipoprotein cholesterol (HDL-C) level in the subject. In some embodiments, the method decreases low-density liproptoen cholesterol (LDL-C) level in the subject. In some embodiments, the method decreases blood glucose level in the subject.

In a second aspect, the disclosure provides a method for increasing high-density lipoprotein cholesterol (HDL-C) level, decreasing low-density lipoprotein cholesterol (LDL-C) level, and/or decreasing blood glucose level in a subject, comprising administering to the subject a composition comprising an effective amount of one or more of the microbial species selected from the group consisting Gokushovirus, Bacillus phage, Escherichia phage, Streptococcus phage, Microvirus, and Candida dubliniesis. In some embodiments of this aspect, the composition comprises Candida dubliniesis.

In some embodiments of the methods described herein, the administering step comprises fecal microbiota transplantation (FMT). In some embodiments, prior to the step of administering, the method comprises identifying a donor subject for the FMT, comprising: (a) analyzing a fecal sample obtained from a candidate subject to detect the presence of the one or more of the microbial species; and (b) determining the candidate subject as the donor subject when the presence of the one or more of the microbial species is detected in the fecal sample. The method can further comprise, prior to step (a), the step of obtaining the fecal sample from a candidate subject. In some embodiments, a fecal sample used in the FMT is obtained from a stool bank. A fecal sample used in the FMT can be administered to the small intestine, the ileum, and/or the large intestine of the subject. In some embodiments, a fecal sample used in the FMT is administered via direct transfer to the GI track. In some embodiments, a fecal sample used in the FMT is formulated for oral administration. For example, the fecal sample is administered before food intake or together with food intake.

In a third aspect, the disclosure provides, a method for determining the risk of a metabolic disease in a subject, comprising detecting, in a biological sample obtained from the subject, the presence of one or more microbial species selected from the group consisting of Bacteroides phage, Pectobacterium phage, Achromobacter phage, Azobacteroides phage, and crAssphage, wherein the presence of the one or more microbial species indicates that the subject is at risk for the metabolic disease. In some embodiments, the abundance of a microbial species selected from the group consisting of Bacteroides phage, Pectobacterium phage, Achromobacter phage, Azobacteroides phage, and crAssphage is at least 50 reads per kilobase of nucleic acid, per million mapped reads (RPKM) (e.g., at least 55, 60, 65, 70, 75, 80, 85, 90, 95, or 100 RPKM). In some embodiments of this aspect, the abundance of the one or more microbial species is higher than the abundance of one or more microbial species selected from the group consisting of Diachasmimorpha longicaudata entomopoxvirus, Megavirus, Oenococcus phage, Saudi moumouvirus, Clostridium botulinum C phage, Emiliania huxleyi virus, and Lausannevirus. In certain embodiments, the abundance of a virus selected from the group consisting of Bacteroides phage, Pectobacterium phage, Achromobacter phage, Azobacteroides phage, and crAssphage is at least 2-fold of the abundance of a virus selected from the group consisting of Diachasmimorpha longicaudata entomopoxvirus, Megavirus, Oenococcus phage, Saudi moumouvirus, Clostridium botulinum C phage, Emiliania huxleyi virus, and Lausannevirus indicates that the subject is at risk for the metabolic disease. In methods described herein, the abundance is determined using metagenomics sequencing or quantitative polymerase chain reaction (qPCR).

In a fourth aspect, the present invention provides a novel method for reducing the risk of a metabolic disease or treating a metabolic disease. The method includes the step of administering to a subject in need thereof a composition comprising an effective amount of one or more of the microbial species selected from the group consisting of Bacillus phage, Bacillus cereus, Bifidobacterium breve, Blautia spp., species under Lachnoclostridium, and viruses named in Table 9.

In some embodiments, the metabolic disease is obesity, pre-diabetes, or type-2 diabetes. In some embodiments, the administering step comprises oral administration or direct delivery to the small intestine, ileum, or large intestine of the subject. In some embodiments, the administering step comprises fecal microbiota transplantation (FMT), for example, the FMT may comprise administration to the subject a composition comprising processed donor fecal material. In some embodiments, the composition comprises no detectable amount of any virus in Table 7 or 8, e.g., no detectable amount of Ugandan cassava brown streak virus. In some embodiments, the treatment results in increased high-density lipoprotein cholesterol (HDL-C) level, decreased low-density lipoprotein cholesterol (LDL-C) level is, and/or decreased blood glucose level in the subject. In some embodiments, bodyweight is reduced in the subject upon receiving the treatment.

In a fifth aspect, the present invention provides a kit for reducing the risk of a metabolic disease or treating a metabolic disease, which includes a first container containing a first a composition comprising an effective amount of a first microbial species selected from the group consisting of Bacillus phage, Bacillus cereus, Bifidobacterium breve, Blautia spp., species under Lachnoclostridium, and viruses in Table 9, and a second container containing a second composition comprising an effective amount of a second (different from the first) microbial species selected from the group consisting of Bacillus phage, Bacillus cereus, Bifidobacterium breve, Blautia spp., species under Lachnoclostridium, and viruses in Table 9.

In some embodiments, either or both of the first and second compositions comprise processed donor fecal material for FMT. In some embodiments, either or both of the first and second compositions are formulated for oral administration. In some embodiments, the kit further includes a third container containing a third composition comprising an effective amount of an antiviral agent inhibiting the viruses in Tables 7 and 8, for example, the antiviral agent inhibits Ugandan cassava brown streak virus.

In a sixth aspect, a method is provided for assessing risk of developing a metabolic disease including obesity among two subjects. The method includes these steps: (1) determining, in a stool sample from a first subject, the level or relative abundance of one or more of the viral species in Tables 7 and 8; (2) detecting the level or relative abundance from step (1) being higher than the level or relative abundance of the same virial species in a stool sample from a second subject; and (3) determining the first subject as having a higher risk of developing a metabolic disease than the second subject. In some embodiments, the one or more viral species comprise Ugandan cassava brown streak virus.

In a seventh aspect, a kit is provided for assessing the likelihood of developing a metabolic disease including obesity in a subject, comprising reagents for detecting one or more of the virial species in Tables 7 and 8. In some embodiments, the reagents comprise a set of oligonucleotide primers for amplification of a polynucleotide sequence from any one of the virial species in Tables 7 and 8. In some embodiments, the one or more viral species comprise Ugandan cassava brown streak virus. In some embodiments, the amplification is PCR, such as quantitative PCR.

In an eighth aspect, methods are provided for determining the risk for obesity and/or type 2 diabetes in a subject, including an obese subject. One method is provided for determining risk for obesity and/or type 2 diabetes risk in an obese test subject, comprising: (a) quantitatively determining the relative abundance of viral species selected from Table 10, Table 13, or Table 16 in a stool sample taken from the test subject; (b) quantitatively determining the relative abundance of viral species selected from Table 10, Table 13, or Table 16 in a stool sample taken from a reference cohort comprising obese subjects, obese with type 2 diabetes subjects, and lean controls; (c) generating decision trees by random forest model using data obtained from (b); (d) running the relative abundance obtained from (a) down the decision trees from (b) to generate a risk score; and (e) determining the test subject with a score greater than 0.5 as having an increased risk for obesity and/or type 2 diabetes, and determining the test subject with a score no greater than 0.5 as having no increased risk for obesity and/or type 2 diabetes.

Another method is provided for determining obesity risk in a test subject, comprising: (1) obtaining from a cohort of obese subjects and lean controls a set of training data by determine the age of subjects and relative abundance of viral species Staphylococcus virus, Phormidium phage, and Costridium virus in stool samples; (2) determining the relative abundance of the viral species in a stool sample taken from the test subject whose risk of obesity is to be determined; (3) comparing the relative abundance of the viral species from step (2) with the training data using random forest model; (4) generating decision trees by random forest from the training data and running the relative abundance from step (2) down the decision trees to generated a risk score; and (5) determining the test subject with a risk score greater than 0.5 as at increased risk for obesity and determining the test subject with a risk score no greater than 0.5 as at no increased risk for obesity. In some embodiments, the viral species further comprise Hepatitis C virus and/or Catovirus.

A further method is provided for determining risk of obesity with type 2 diabetes in a test subject, comprising: (1) obtaining from a cohort of obese with type 2 diabetes subjects and lean controls a set of training data by determine the age of subjects and relative abundance of viral species Achromobacter phage, Oenococcus phage, and Geobacillus phage in stool samples; (2) determining the relative abundance of the viral species in a stool sample taken from the test subject whose risk of obesity with type 2 diabetes is to be determined; (3) comparing the relative abundance of the viral species from step (2) with the training data using random forest model; (4) generating decision trees by random forest from the training data and running the relative abundance from step (2) down the decision trees to generated a risk score; and (5) determining the test subject with a risk score greater than 0.5 as at increased risk for obesity with type 2 diabetes and determining the test subject with a risk score no greater than 0.5 as at no increased risk for obesity with type 2 diabetes. In some embodiments, the viral species further comprise one or more of Mycoplasma phage, Klosneuvirus, and Fowl aviadenovirus.

An additional method is provided for determining type 2 diabetes risk in an obese test subject, comprising: (1) obtaining from a cohort of obese with type 2 diabetes subjects and obese controls a set of training data by determine the age of subjects and relative abundance of viral species Oenococcus phage and Bradyrhizobium phage in stool samples; (2) determining the relative abundance of the viral species in a stool sample taken from the test subject whose type 2 diabetes risk is to be determined; (3) comparing the relative abundance of the viral species from step (2) with the training data using random forest model; (4) generating decision trees by random forest from the training data and running the relative abundance from step (2) down the decision trees to generated a risk score; and (5) determining the test subject with a risk score greater than 0.5 as at increased risk for type 2 diabetes and determining the test subject with a risk score no greater than 0.5 as at no increased risk for type 2 diabetes. In some embodiments, the viral species further comprise one or more of Phormidium phage, Heliothis zea nudivirus, and Achromobacter phage.

### BRIEF DESCRIPTION OF THE DRAWINGS

FIGS. 1A and 1B: The alpha diversity of gut virome in obesity and T2DM subjects. (A) Chao1 (richness) and (B) Shannon (diversity) were significantly reduced in obese with T2DM subjects. Statistical significance was determined by Wilcoxon rank sum test. P value *<0.05; **<0.01.

FIGS. 2A-2D: Gut viral-types is different in obesity, obesity with T2DM and control. (A) Proportions of the 4 viral-types between groups. Statistical significance was determined by Fisher's exact test, p=0.0014. (B) Chao1 (richness) and (C) Shannon (diversity) index between viral-types. Statistical significance was determined by Wilcoxon rank sum test. P value *<0.05; **<0.01; ***<0.001. (D) Relative abundance of the differential viral species between viral-types. Differences in abundance were detected using Lefse (Linear discriminant analysis Effect Size). Only the differential species with largest effect size were selected for visualization.

FIGS. 3A-3D: Virome enterotypes in health and its correlation with HDL-cholesterol. (A) Two virome enterotypes were identified. Virome enterotype clustering was based on partition around medoids (PAM) algorithm and principal cooridinates analysis (PCoA) on the viral community structures of all subjects. (B) Differentially enriched viral species between Virome Enterotypes 1 and 2. Discriminant species were determined by LefSE analysis with FDR correction. Only those taxa with adjusted p values<0.05 and effect size >2 are plotted. (C) Comparison of the gut virome a diverisity indices (diversity, richness, and evenness) between Virome Enterotypes 1 and 2. Between-group comparisons and statistical significance were determined by t test, ****p<0.0001. (D) Concentration of high-density lipoprotein-cholesterol (HDL-C) in blood with respect to Virome Enterotype. Between-group comparison and statistical significance were determined by t test, *p<0.05.

FIGS. 4A-4E: Overview of the study populations and their gut mycobiome. (A) Geographical distribution of the studied populations. Yunnan province and Hong Kong (China) were the sampling regions. In Hong Kong, all recruited subjects were urban residents and ethnically Chinese Han. In Yunnan, the rural populations corresponding to the ethnic groups Han, Zang, Miao, Bai, Dai, and Hani resided in different rural districts circumferential of Kunming (the provincial capital of Yunnan province, an urban city). All enrolled urban ethnic groups in Yunnan, including Han, Zang, Miao, Bai, Dai, and Hani, cohabited in Kunming. (B) The number of subjects recruited in this study. A total of 942 Chinese were sampled (n=61, Hong Kong subjects; and n=881, Yunnan subjects). (C) An overview of study design, including mycobiome and bacteriome profilings, metadata questionnaire investigation, and blood biochemical measurements. (D) Variations in gut mycobiome composition at the family level across all study subjects, plotted according to the relative abundance of predominant gut fungi. (E) Family-level gut mycobiome compositions plotted according to geographic region (Hong Kong versus Yunnan), ethnicity (Han, Zang, Miao, Bai, Dai, and Hani), and residency (rural versus urban).

FIGS. 5A and 5B: Variations in the gut bacterial microbiome across study populations. (A) Variations in the gut bacterial microbiome (bacteriome) at the phylum level across all study subjects, plotted according to the relative abundance of the gut bacteria phyla. (B) Phylum-level gut bacteriome compositions plotted with respect to geographical region (Hong Kong versus Yunnan), ethnicity (Han, Zang, Miao, Bai, Dai, and Hani), and residency (rural versus urban).

FIGS. 6A-6C: Identified mycobiome covariates and their effect sizes in gut myocbiome variation. (A) The effect of size of metadata variables in human gut mycobiome variation. Mycobiome covariates were identified via envfit (vegan) and those with statistical significance (FDR adjusted p<0.05) were colored based on metadata category in FIG c; p<0.01**. (B) Pie chart shows the fraction of microbial variation explained by all captured metadata variables. (C) Combined effect size of mycobiome covariates pooled in predefined categories with covariate distance-based selection.

FIGS. 7A and 7B: Dietary differences across populations and their effect in mycobiome variations. (A) Differences in consumption structure of dietary components across different Chinese populations (Hong Kong versus Yunnan populations, six ethnic groups, and rural versus urban residents). Correlation of dietary components with each population was calculated with pairwise Chi-square test and Crammer's V correlation estimation. Only significant correlations with FDR adjusted p<0.05 were shown with color intensified according to correlation coefficient. (B) The effect size of dietary components in mycobiome composition variation. Dietary variables are sorted according to their effect size. Only those with statistical significance (FDR adjusted p<0.05) were plotted.

FIGS. 8A-8C: Variations in the α diversity of human gut mycobiome according to geography, ethnicity and rural versus urban residency. The fecal fungal diversity (A) and richness (B) (as measured in Simpson diversity and Chao1 richness index respectively) were plotted and compared across all ethnic subgroups, and between rural and urban subjects with respect to each ethnic group. For box plots, the boxes extend from the 1st to 3rd quartile (25^{th} to 75^{th} percentile), with the median depicted by a horizontal line. Across-population comparisons were conducted between the base mean of group of interest and that of all groups, statistical significance was determined by wilcox rank sum test with Holm-Bonferrroni adjustment of p values, ^{#}p<0.05, ^{##}p<0.01, ^{###}p<0.001, ^{####}p<0.0001. Statistical significance between rural and urban populations for each individual ethnic group was determined by Mann-Whitney test, *p<0.05, **p<0.01. (C) Presence-absence heatmap of fungal species in the gut mycobiota of different populations.

FIG. 9: The β diversity of mycobiomes with respect to urbanisation, geography, and ethnicity. The β diversities of mycobiomes were calculated as the Aitchison distance between individual mycobiomes. The mycobiome species compositional data were CLR transformed. Between-group comparisons and statistical significance were determined by t test, ****p<0.0001.

FIGS. 10A-10C: Variations in the mycobiome composition according to geography, ethnicity and rural versus urban residency. The mycobiomes were analyzed and plotted via principal component analysis (PCA) based upon the Aitchison distance between species-level mycobiome compositions. The mycobiome compositional data was centered log-ratio (CLR) transformed prior to PCA analysis. (A) Mycobiome variations with respect to rural versus urban residency. Comparison of subject mycobiome distributions on PC1 between rural and urban subjects and statistical significance were determined by t test, ****p<0.0001. (B) Mycobiome variations between Hong Kong and Yunnan subjects. Comparison of subject mycobiome distributions on PC1 between Hong Kong and Yunnan and statistical significance were determined by t test, **p<0.01. (C) Mycobiome variations across ethnicity. Comparison of subject mycobiome distributions on PC1 between ethnicities and statistical significance were determined by one-way anova with Tukey's HSD test, and Holm-Bonferrroni p adjustment, ^{#}p<0.05, ^{##}p<0.01. For box plots, the boxes extend from the 1st to 3rd quartile (25^{th} to 75^{th} percentile), with the median depicted by a horizontal line.

FIGS. 11A and 11B: Urbanisation significantly shifted the mycobiome configuration of all ethnic groups in Yunnan. (A) db-RDA analysis was conducted for each ethnicity in Yunnan. Capscale test was used to determine statistical significance, **p<0.01, ***p<0.001. (B) species presence heatmap of the differential fungal taxa between rural and urban mycobiomes. Differentially enriched fungal taxa between Yunnan rural and urban mycobiomes were determined by LefSE analysis with FDR correction (only those differential taxa with adjusted p values<0.05 and effect size >2 are shown). Taxa color-coated in red denote taxa enriched in urban subjects, while those color-coated in green denote taxa enriched in rural subjects.

FIG. 12: Phylogenetic illustration of the discriminant fungal taxa between the gut mycobiomes of the Hong Kong and Yunnan populations. Differentially enriched fungal taxa between Hong Kong and Yunnan mycobiomes were determined by LefSE analysis with FDR correction (only those differential taxa with adjusted p values<0.05 and effect size >2 are shown). Taxa color-coated in red denote taxa enriched in Hong Kong subjects, while those color-coated in green denote taxa enriched in Yunnan subjects.

FIGS. 13A and 13B: Variations in gut mycobiome across Yunnan populations, according to population district residency. The mycobiomes were analyzed and plotted via principal component analysis (PCA) based upon the Aitchison distance between species-level mycobiome compositions. (A) Mycobiome variations as a function of district residency in Yunnan, viewed in PCA plot. (B) Population mycobiome vatiation on PC1 were plotted and statistically tested via one-way anova and Tukey's HSD test, with Holm-Bonferrroni p adjustment, *p<0.05, ***p<0.001, ****p<0.0001. For box plots, the boxes extend from the 1st to 3rd quartile (25^{th} to 75^{th} percentile), with the median depicted by a horizontal line.

FIGS. 14A-14E: Ethnicity-specific fungal species in the Yunnan populations. Differentially present fungal species associated with specific ethnic groups in Yunnan populations were determined by MaAsLin2 analysis with Holm-Bonferrroni adjustment of p values, ****p<0.0001. (A and B) fungal species identified as highly present inethnic Hani; (C-E) fungal species identified highly present in ethnic Zang. For box plots, the boxes extend from the 1st to 3rd quartile (25^{th} to 75^{th} percentile), with the median depicted by a horizontal line.

FIGS. 15A and 15B: Correlation between gut fungal species and subject blood biochemical parameters. (A) Correlations between CLR transformed species abundance and blood biochemical measurements were calculated through Pearson correlation test. Correlation coefficient was calculated, whilst statistical significance was determined for all pairwise comparisons. Only statistically significant correlations were shown. (B) The relative abundance of Candida dubliniensis in the gut mycobiome of obese versus lean individuals. Statistical significance was determined by Mann-Whitney test, *p<0.05. For box plots, the boxes extend from the 1st to 3rd quartile (25^{th} to 75^{th} percentile), with the median depicted by a horizontal line.

FIGS. 16A and 16B: Correlations between gut mycobiome and bacteriome. (A) linear regression and correlation between the richness (Chao1) of gut mycobiome and bacteriome. Pearson correlation test was performed for coefficient estimation with statistical significance determination. (B) Correlations among gut fungal and bacterial taxa. Correlations between taxa were calculated through SpiecEasi correlation test. Correlation coefficient was calculated, whilst statistical significance was pairwise determined for all comparisons. Only statistically significant correlations with |correlation coefficient|>0.2 were plotted. The correlation network was visualized via Cytoscape. The size of node, corresponding to microbial species, was proportional to the number of significant inter-species correlations.

FIG. 17. Alterations of gut virome in obese subjects compared with lean controls. (A) Chao1 richness and (B) Shannon diversity for gut virome between obese subjects and lean controls at contig level. (C) Principal Coordinates Analysis (PCoA) of Bray-Curtis distance showing the stratification of obese subjects from lean controls by gut virome after adjusted covariant of T2DM. Statistical significance was determined by Wilcoxon rank sum test.

FIG. 18. Gut viral taxonomic distribution in obese subjects and lean controls. (A) Relative abundance of gut viral orders. HK: Hong Kong, HK: KunMing. (B) Relative abundance of the differential viral species between obese subjects and lean controls. Differences in abundance were detected using Microbiome Multivariable Association with Linear Models (MaAslin2) and corrected for confounders including age, gender, alcohol intake, smoking, T2DM and cohort. Log transformation of the relative abundance were shown in the boxplot.

FIG. 19. Alterations of gut virome in ObT2, Ob compared with lean controls. (A) Chao1 richness and (B) Shannon diversity for gut virome between ObT2, Ob and lean controls at contig level. Wilcoxon rank sum test was used to determine statistically significant difference between groups. (C) Principal Coordinates Analysis (PCoA) of Bray-Curtis distance showing the stratification of ObT2 from lean controls and Ob by gut virome. (D) Relative abundance of the differential viral species between ObT2 and lean controls. Differences in abundance were detected using MaAslin2 and corrected for confounders including age, gender, alcohol intake, smoking and cohort. The log transformation of fold change for relative abundance compared to mean relative abundance of lean control subjects were shown in the heatmap. Ob: obese subjects without T2DM; ObT2: obese subjects with T2DM.

FIG. 20. Decreased number of inter-kingdom ecological correlations between gut virome and bacteriome in obese subjects compared with lean controls. Correlation coefficients were estimated and corrected for compositional effects using the SparCC algorithm. Only the taxa with relative abundance >1e⁻⁴ were selected for SparCC calculation. A subset of correlations with coefficient strengths of >0.5 or <−0.5 and p value adjusted by false discovery rate (FDR)<0.05 were regards significant and selected to visualization. The viral species were classified by family level in columns and bacteria species were classified by phylum level in rows.

FIG. 21. ROC of the machine learning model 1 trained to predict obesity based on relative abundance of Staphylococcus virus, Phormidium phage, Costridium virus (red), Staphylococcus virus, Phormidium phage, Costridium virus, age (orange), Staphylococcus virus, Phormidium phage, Costridium virus, age, Hepatitis C virus (green) and Staphylococcus virus, Phormidium phage, Costridium virus, age, Hepatitis C virus, Catovirus (blue)

FIG. 22. Risk score of a 34-year-old female subject compared to obese subjects and lean controls using 6 markers: Staphylococcus virus, Phormidium phage, Costridium virus, age, Hepatitis C virus and Catovirus. The score of the subject was 0.733 using Model 1 and therefore the subject was deemed to have a higher risk for obesity. This subject had BMI 41.5 (obese).

FIG. 23. ROC of the machine learning model 2 trained to predict obesity with type 2 diabetes based on relative abundance of Achromobacter phage, Oenococcus phage, Geobacillus phage (red), Achromobacter phage, Oenococcus phage, Geobacillus phage, Mycoplasma phage (orange), Achromobacter phage, Oenococcus phage, Geobacillus phage, Mycoplasma phage, Klosneuvirus (green) and Achromobacter phage, Oenococcus phage, Geobacillus phage, Mycoplasma phage, Klosneuvirus, Fowl aviadenovirus (blue)

FIG. 24. Risk score of a 57-year-old male subject compared with obese subjects with T2DM (n=29) and lean controls (n=40) using 6 markers: Achromobacter phage, Oenococcus phage, Geobacillus phage, Mycoplasma phage, Klosneuvirus and Fowl aviadenovirus. The score of the subject was 0.637, and therefore the subject was deemed to have a medium risk of obesity combined with T2DM. The subject has a BMI of 35.6 and was diagnosed with type 2 diabetes

FIG. 25. ROC of the machine learning model 3 trained to predict type 2 diabetes in subjects with known obesity based on relative abundance of age, Oenococcus phage, Bradyrhizobium phage (red); age, Oenococcus phage, Bradyrhizobium phage, Phormidium phage (orange); age, Oenococcus phage, Bradyrhizobium phage, Phormidium phage, Heliothis zea nudivirus (green); and age, Oenococcus phage, Bradyrhizobium phage, Phormidium phage, Heliothis zea nudivirus, Achromobacter phage (blue)

FIG. 26. Risk score of a 46-year-old male subject compared with obese subjects with T2DM (n=29) and obese subject (n=21) using 6 markers: age, Oenococcus phage, Bradyrhizobium phage, Phormidium phage, Heliothis zea nudivirus and Achromobacter phage. The score of the subject was 0.864, and therefore the subject was deemed to have a higher risk of obesity combined with T2DM. This subject had a BMI of 37 and was diagnosed with T2DM.

FIG. 27. Alterations of gut virome in obese subjects compared with lean controls in Hong Kong (HK) and KunMing (KM). (A) Chao1 richness and (B) Shannon diversity for gut virome between obese subjects and lean controls at contig level. (C) Principal Coordinates Analysis (PCoA) of Bray-Curtis distance showing the stratification of obese subjects from lean controls by gut virome after adjusted covariant of T2DM. Statistical significance was determined by Wilcoxon rank sum test. p value <0.05 were regards significant.

FIG. 28. No significant association between gut viral alpha diversity, gender and age. (A) Chao1 richness and (B) Shannon diversity correlate with age in obese subjects and lean controls. Statistical significance was determined by linear regression. (C) Chao1 richness and (D) Shannon diversity between genders in obese subjects and lean controls. Statistical significance was determined by Wilcoxon rank sum test. p value <0.05 were regards significant.

FIG. 29. Alterations of gut virome in ObT2, Ob compared with lean controls in Hong Kong (HK) and KunMing (KM). (A) Chao1 richness and (B) Shannon diversity for gut virome. Kruskal-Wallis test and Wilcoxon rank sum text was used to determine statistically significant difference between groups. Ob: obese subjects without T2DM; ObT2: obese subjects with T2DM.

FIG. 30. Association between medications and gut viral alpha diversity in ObT2. (A) Chao1 richness and (B) Shannon diversity between medication users and non-users. Medication includes Metformin, Non-Steroidal Anti-Inflammatory Drugs (NASIDs), Proton-pump inhibitors (PPIs), Statin and Sulfonylureas (SUs). Statistical significance was determined by Wilcoxon rank sum test. p value <0.05 were regards significant. ObT2: obese subjects with T2DM. Y: medication users. N: Non-medication users.

FIG. 31. Random forest features sorting by MeanDecreaseAccuracy in prediction models. (A) Ob vs Lean controls. (B) ObT2 vs Lean controls. (C) ObT2 vs Ob. Feature includes relative abundance of gut viral species and metadata includes age, gender, alcohol intake, smoking, hypertension. Ob: obese subjects without T2DM; ObT2: obese subjects with T2DM.

FIG. 32. Decreased number of significant inter-kingdom ecological correlations between gut virome and bacteriome in Ob and ObT2 compared with lean controls. Correlation coefficients were estimated and corrected for compositional effects using the SparCC algorithm. Only the taxa with relative abundance >1e-4 were selected for SparCC calculation. A subset of correlations with coefficient strengths of >0.5 or <−0.5 and p value adjusted by false discovery rate (FDR)<0.05 were regards significant and selected to visualization. The viral species were classified by family level in columns and bacteria species were classified by phylum level in rows. Ob: obese subjects without T2DM; ObT2: obese subjects with T2DM.

### DEFINITIONS

The term “fecal microbiota transplantation (FMT)” or “stool transplant” refers to a medical procedure during which fecal matter containing live fecal microorganisms (bacteria, fungi, viruses, and the like) obtained from a healthy individual is transferred into the gastrointestinal tract of a recipient to restore healthy gut microflora that has been disrupted or destroyed by any one of a variety of medical conditions, for example, excess body weight or obesity and its related disorders. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing processed (e.g., dried and frozen or lyophilized) fecal material.

The term “inhibiting” or “inhibition,” as used herein, refers to any detectable negative effect on a target biological process, such as RNA/protein expression of a target gene, the biological activity of a target protein, cellular signal transduction, cell proliferation, and the like. Typically, an inhibition is reflected in a decrease of at least 10%, 20%, 30%, 40%, 50%, 60%, 70%, 80%, 90% or greater in the target process (e.g., growth or proliferation of a microorganism of certain species, for example, one or more of Bacteroides phage, Pectobacterium phage, Achromobacter phage, Azobacteroides phage, crAssphage and the viral species shown in Table 7 or 8), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” “reduction,” “decrease,” “decreasing,” “lower,” and “less” are used in a similar fashion in this disclosure to refer to decreases to different levels (e.g., at least 10%, 20%, 30%, 40%, 50%, 60%, 70%, 80%, 90% or greater decrease compared to a control level, i.e., the level before suppression) up to complete elimination of a target biological process or signal. On the other hand, terms such as “activate,” “activating,” “activation,” “increase,” “increasing,” “promote,” “promoting,” “enhance,” “enhancing,” “enhancement,” “higher,” and “more” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level (before activation), for example, the control level of one or more of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and the viral species shown in Table 9) in a target process or signal. In contrast, the term “substantially the same” or “substantially lack of change” indicates little to no change in quantity from a comparison basis (such as a standard control value), typically within ±10% of the comparison basis, or within ±5%, 4%, 3%, 2%, 1%, or even less variation from the comparison basis.

The term “anti-bacterial/viral agent” refers to any substance that is capable of inhibiting, suppressing, or preventing the growth or proliferation of bacterial/viral species, respectively, especially those of shown in Table 1, 7, or 8. Known agents with anti-bacterial or anti-viral activity include generic inhibitors such as various antibiotics that generally suppress the proliferation of a broad spectrum of bacterial species as well as agents such as antisense oligonucleotides, small inhibitory RNAs, and the like that can inhibit the proliferation of specific bacterial or viral species. The term “anti-bacterial/viral agent” is similarly defined to encompass both agents with broad spectrum activity of killing virtually all species of bacteria or viruses and agents that specifically suppress proliferation of target bacteria/virus species. Such specific anti-bacterial/viral agent may be short polynucleotide in nature (e.g., a small inhibitory RNA, microRNA, miniRNA, lncRNA, or an antisense oligonucleotide) that is capable of disrupting the expression of a key gene in the life cycle of a target bacterial or viral species and is therefore capable of specifically suppressing or eliminating the species only without substantially affecting other closely related bacterial or viral species.

“Percentage relative abundance,” when used in the context of describing the presence of a particular viral or bacterial species (e.g., any one of those shown in of Tables 1, 2, and 7-9) in relation to all viral or bacterial species present in the same environment, refers to the relative amount of the viral or bacterial species out of the amount of all viral or bacterial species as expressed in a percentage form. For instance, the percentage relative abundance of one particular bacterial species can be determined by comparing the quantity of DNA specific for this species (e.g., determined by quantitative polymerase chain reaction) in one given sample with the quantity of all bacterial DNA (e.g., determined by quantitative polymerase chain reaction (PCR) and sequencing based on the 16s rRNA sequence) in the same sample.

“Absolute abundance,” when used in the context of describing the presence of a particular viral or bacterial species (e.g., any one of those shown in of Tables 1, 2, and 7-9) in the feces, refers to the amount of DNA derived from the viral or bacterial species out of the amount of all DNA in a fecal sample. For instance, the absolute abundance of one bacterium can be determined by comparing the quantity of DNA specific for this bacterial species (e.g., determined by quantitative PCR) in one given sample with the quantity of all fecal DNA in the same sample.

“Total bacterial/viral load” of a fecal sample, as used herein, refers to the amount of all bacterial or viral DNA, respectively, out of the amount of all DNA in the fecal sample. For instance, the absolute abundance of bacteria can be determined by comparing the quantity of bacteria-specific DNA (e.g., 16s rRNA determined by quantitative PCR) in one given sample with the quantity of all fecal DNA in the same sample.

As used herein, the term “metabolic disease” refers to a disease, disorder, or syndrome that is related to a subject's metabolism, such as breaking down carbohydrates, proteins, and fats in food to release energy, and converting chemicals into other substances and transporting them inside cells for energy utilization and/or storage. Some symptoms of a metabolic disease include high blood glucose, low high density lipoprotein cholesterol (HDL-C), high low density lipoprotein cholesterol (LDL-C), high serum triglycerides, high fasting insulin levels, elevated fasting plasma glucose, abdominal (central) obesity, and elevated blood pressure. Metabolic diseases also include diseases where the subjects have difficulties digesting and/or absorbing certain foods, as well as diseases where the subjects have allergic reactions towards certain foods. Metabolic diseases in a subject can be caused by a number of factors, such as, but not limited to, environmental conditions, personal and/or lifestyle choices, and/or genetic makeups in the subject. Metabolic diseases increase the risk of developing other diseases, such as cardiovascular disease and hypertension. In the present disclosure, metabolic diseases include, but are not limited to, obesity, type-1 diabetes, and type-2 diabetes.

The term “overweight” is used to describe a subject of excessive body weight and having a body mass index (BMI) greater than 25. Encompassed with this term is “obese” or “obesity,” which describes a condition in which the suffer has a BMI greater than 30.

The term “treat” or “treating,” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition, including facilitation of patient recovery from the condition.

As used herein, the term “prevent” or “preventing” includes providing prophylaxis with respect to the occurrence or recurrence of a disease or medical condition in a subject that may be predisposed to the disease/condition but has not yet been diagnosed with the disease or condition.

As used herein, the term “pharmaceutical composition” refers to a medicinal or pharmaceutical formulation that contains an active ingredient as well as excipients and diluents to enable the active ingredient suitable for the method of administration. The pharmaceutical composition of the present invention includes pharmaceutically acceptable components that are compatible with the microbial species in the composition.

The term “effective amount,” as used herein, refers to an amount of a substance that produces a desired effect (e.g., an inhibitory or suppressive effect on the growth or proliferation of one or more detrimental viral species (e.g., the viral species shown in Table 1, 7, or 8) for which the substance (e.g., an anti-viral agent) is used or administered. The effects include the prevention, inhibition, or delaying of any pertinent biological process during viral proliferation to any detectable extent. The exact amount will depend on the nature of the substance (the active agent), the manner of use/administration, and the purpose of the application, and will be ascertainable by one skilled in the art using known techniques as well as those described herein. In another context, when an “effective amount” of one or more beneficial or desirable viral or bacterial species (e.g., those listed in Table 2 or Table 9) are artificially introduced into a composition intended to be introduced into the gastrointestinal tract of a patient, e.g., to be used in FMT, it is meant that the amount of the pertinent viral species being introduced is sufficient to confer to the recipient health benefits such as reduced recovery time or reduced needs for therapeutic intervention for a pertinent disorder such as excessive body weight or obesity or metabolic disease, including but not limited to medication (such as an appetite suppressant) and any of the variety of therapies such as behavior and communication therapy, educational therapy, family therapy, speech or physical therapy, and the like.

As used herein, the term “about” denotes a range of value that is +/−10% of a specified value. For instance, “about 10” denotes the value range of 9 to 11 (10+/−1).

### DETAILED DESCRIPTION OF THE INVENTION

### I. Introduction

The invention provides novel methods for achieving weight loss and treating or preventing metabolic diseases in individuals by modifying their bacteria and/or virus profile in their gastrointestinal tract as well as for assessing the likelihood of developing obesity and/or metabolic diseases in individuals by way of fecal microbiota transplantation (FMT) treatment. During their studies, the present inventors discovered that the presence and relative abundance of certain viral and/or bacterial species alter significantly in the gastrointestinal tract of overweight especially obese individuals as well as those have developed a metabolic disease such as type 2 diabetes. For example, the presence and abundance of viral species shown in Table 1, 7, or 8 is found to be at an elevated level in the gastrointestinal tract of those who suffer from obesity and/or a metabolic disease such as type 2 diabetes. On the other hand, the level or relative abundance of certain viral species (such as those shown in Table 2 or 9) in individuals' stool samples has been observed to correlate with a reduced risk of developing obesity and/or metabolic diseases. Thus, the results of this study provide useful tools for facilitating weight loss efforts in overweight/obese individuals, for reducing risk for a metabolic disease or treating a metabolic disease in patients as well as for assessing the risk for obesity and/or for a metabolic disease such as type 2 diabetes among individuals.

### II. FMT Donor/Recipient Selection and Preparation

Overweight individuals suffer from a disrupted state of GI tract microflora are considered as recipients for FMT treatment in order to restore the normal healthy profile for microorganisms. As revealed by the present inventors, overweight or obese individuals, especially those who suffer from metabolic disease such as type 2 diabetes, tend to have a depressed level of viral species such as Bacillus phage, Bacillus cereus, Bifidobacterium breve, Blautia spp., a species under Lachnoclostridium, and those shown in Table 9 in their GI tract, a FMT donor whose fecal material contains an higher than average level of one or more of these viral species is favored as particularly advantageous for the purpose of a subsequent FMT therapy for bodyweight reduction and prevention or treatment of a metabolic disease. For example, a desirable donor may preferably have higher than about 0.01%, 0.02%, 0.05%, 0.10%, 0.20%, 0.40%, 0.50%, 0.60%. 0.80%, 1.0%, 2.0%, 3.0%, 4.0%, 5.0%, 6.0%, 7.0%, 8.0%, 8.5%, 9.0%, or higher of total virus in relative abundance for each of these viral species in his stool sample.

Similarly, for use of other preferable viral or bacterial species such as Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Escherichia phage, Streptococcus phage, Microvirus, and Candida dubliniesis in accordance with the methods of the present invention, a desirable donor may preferably have higher than about 0.01%, 0.02%, 0.05%, 0.10%, 0.20%, 0.40%, 0.50%, 0.60%. 0.80%, 1.0%, 2.0%, 3.0%, 4.0%, 5.0%, 6.0%, 7.0%, 8.0%, 8.5%, 9.0%, or higher of total virus or bacteria in relative abundance for each of these viral or bacterial species in his stool sample.

Fecal matter used in FMT is obtained from a healthy donor and then processed into appropriate forms for the intended means of delivery in the upcoming FMT procedure. While a healthy individual from the same family or household of the recipient often serves as donor, in practicing the present invention the donor microorganism profile is an important consideration and may favor the choice of an unrelated donor instead. The process of preparing donor material for transplant includes steps of drying, freezing or lyophilizing, and formulating or packaging, depending on the precise route of delivery to recipient, e.g., by oral ingestion or by rectal deposit.

Various methods have been reported in the literature for determining the levels of all viral or bacterial species in a sample, for example, amplification (e.g., by PCR) and sequencing of bacterial polynucleotide sequence taking advantage of the sequence similarity in the commonly shared 16s rRNA sequence. On the other hand, the level of any given bacterial species may be determined by amplification and sequencing of its unique genomic sequence. A percentage abundance is often used as a parameter to indicate the relative level of a bacterial species in a given environment.

### III. Treatment Methods by Modulating Viral or Bacterial Level

The discovery by the present inventors reveals the direct correlation between an individual's risk of developing obesity or a metabolic disease such as type 2 diabetes and the presence and relative abundance of certain viral or bacterial species (e.g., those shown in Table 1, 2, 7, 8, or 9) in the individual's GI tract. This revelation enables different methods for treating overweight/obese individuals for weight loss, especially for treating those who have already developed obesity, to reduce their chances of further developing a metabolic disease such as type 2 diabetes, by adjusting or modulating the level of these viral species as well as certain related bacterial and viral species in these individuals' GI tract via, e.g., a subsequent FMT procedure or an alternative means, to deliver to the patients' GI tract an effective amount of one or more of viral or bacterial species such as Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., a species under Lachnoclostridium, and those shown in Table 9.

When a proposed FMT donor whose stool is tested and found to contain an insufficient level of one or more of the beneficial viral or bacterial species such as those shown in Table 2 or 9 or named above (e.g., each is less than about 0.01%, 0.05%, 0.10%, 0.20%, 0.40%, 0.50%, 0.80%, 1.0%, 2.0%, 3.0%, 4.0%, 5.0%, 6.0%, 7.0%, or 8.0% of total virus or bacteria in the stool sample), the proposed donor is deemed as an unsuitable donor for FMT intended to treat overweight/obese individuals for the purpose of successful reduction of risk for developing a metabolic disease. Otherwise he may be disqualified as a donor in favor of anther individual whose stool sample exhibits a more favorable bacterial and/or viral profile, and his fecal material should not be immediately used for FMT due to the lack of prospect of conferring such beneficial health effects unless the stool material is adequately modified. In these cases of expected lack of weight loss benefits from FMT treatment can be readily improved in view of the inventors' discovery, for example, one or more of the viral or bacterial species such as those shown in Table 2 or 9 or named in the previous paragraph may be introduced from an exogenous source into a donor fecal material so that the level of the viral or bacterial species in the fecal material is increased (e.g., to reach at least about 0.01%, 0.02%, 0.05%, 0.10%, 0.20%, 0.40%, 0.50%, 0.60%. 0.80%, 1.0%, 2.0%, 3.0%, 4.0%, 5.0%, 6.0%, 7.0%, 8.0%, 8.5%, 9.0%, or 10% of total virus or bacteria in the fecal material) before it is processed for use in FMT for the treatment of overweight or obese individuals for the purpose of bodyweight reduction and treatment/prevention of a metabolic disease.

As an alternative, the beneficial viral or bacterial species (e.g., one or more of those shown in Table 2 or 9 as well as others named above and herein) may be obtained from a virus or bacteria culture in a sufficient quantity and then formulated into a suitable composition, which is without any fecal material taken from a donor, for delivery into the gut of an overweight/obese patient or a patient diagnosed with a metabolic disease. Similar to FMT, such composition can be introduced into a patient by oral, nasal, or rectal administration.

Immediately upon completion of the step of introducing an effective amount of the desired viral or bacterial species into a patient's GI tract (e.g., via an FMT procedure), the recipient may be further monitored by continuous testing of the level or relative abundance of the viral or bacterial species in the stool samples on a daily basis for up to 5 days post-procedure while the patient's bodyweight as well as the general health status of the patient are also being monitored in order to assess treatment outcome and the corresponding levels of relevant virus or bacteria in the recipient's GI tract: the level of virus or bacterial species (e.g., one or more of those shown in Table 2 or 9 or those named above) may be monitored in connection with observation of health benefits achieved in association with bodyweight reduction and prevention of progression of a metabolic disease such as improvement in blood glucose, cholesterol, and triglyceride levels.

### IV. Assessing Risk for Obesity and/or Metabolic Disease and Corresponding Treatment

The present inventors also discovered that the altered level of certain virus species can indicate the prospect or likelihood of an individual later develop a metabolic disease, including from obesity to type 2 diabetes: they revealed the correlation between increased level of certain viral species (e.g., those shown in Table 1, 7, or 8) or decreased level of other viral species (e.g., those shown in Table 2 or 9) in individuals' stool samples and the likelihood of later developing obesity and/or a metabolic disease in these patients. Further, the level or relative abundance of certain virial species have been revealed to indicate an individual's prospect or likelihood for later developing a metabolic disease (including obesity and type 2 diabetes) when properly calculated using certain specified mathematic tools.

For example, when stool samples taken from two or more individuals, the level or relative abundance of viral species in Table 1, 2, 7, 8, or 9 and others named above and herein in the samples may be determined, for example, by PCR especially quantitative PCR. For the viral species listed in Table 1, 7, or 8 and others named above and herein, a lower level found in a patient's stool sample indicates a lower likelihood for the patient to later develop obesity or a metabolic disease; conversely, a higher level indicates a higher risk for obesity or metabolic diseases in the individual. On the other hand, for the viral species listed in Table 2 or 9 and others named above and herein, a higher level found in a patient's stool sample indicates a lower likelihood for the patient to later develop obesity or a metabolic disease; conversely, a lower level indicates a higher risk for obesity or metabolic diseases in the individual. In the event that the level of multiple species (e.g., those listed in Table 1, 2, 7, 8, or 9) are measured and compared, the determination of the likelihood of weight loss success is made based on the indication from the majority of the pertinent viral species measured.

Once the assessment for obesity/metabolic disease is made, for example, an individual especially an overweight or obese individual is deemed to have an increased risk for later developing a metabolic disease, appropriate treatment steps can be taken as a measure prevent/treat or reduce the risk of metabolic disease such as type 2 diabetes. To achieve this goal several measures can be taken, for example, the patient may be given compositions that comprise an effective amount of one or more of the viral species listed in Table 2 or 9 or other viral or bacterial species named above either by FMT or by an alternative administration method, such that the viral and/or bacterial profiled in the patient's GI tract will be modified to one that is favorable for weight loss as well as preventing the onset or progression of metabolic diseases.

### Administration

A fecal sample containing one or more microbial species for use in an FMT procedure obtained from a donor subject can be processed and administered to a subject in need to prevent or treat a metabolic disease in the subject. In some embodiments, the fecal sample can be processed and formulated for oral administration. For example, the subject can ingest the processed fecal sample before food intake or together with food intake. In other examples, the processed fecal sample can be administered by direct transfer to the GI track. For example, the subject can undergo FMT where the processed fecal sample is delivered to the small intestine, the ileum, and/or the large intestine of the subject. In other embodiments, the processed fecal sample can also be formulated for local delivery by suppository, such as via rectal administration. In further embodiments, a processed sample containing one or more microbial species can also be delivered via nasal intubation.

The donor subject can be someone who is healthy and does not have a metabolic disease and/or is not at risk for developing a metabolic disease. For example, frozen or fresh stool can be freshly prepared on the day of administration using stool from a single donor subject or using stools from a mixture of multiple donor subjects. Fecal samples can be diluted with sterile saline (0.9%). This solution can then be blended and strained with filter. The resulting supernatant can then be used directly as fresh FMT solution or stored as frozen FMT solution to be used on another day.

The processed fecal sample can be formulated for oral delivery. The following is an example of capsulized, freeze-dried fecal microbiota. Processing is carried out under aerobic conditions. A fecal suspension is generated in normal saline without preservatives using a commercial blender. The slurry is centrifuged at 200 g for 10 minutes to remove debris. The separate fraction was centrifuged at 6,000×g for 15 min and re-suspended to one-half (0.5 mL) the original volume in trehalose (at 5% and 10% concentrations) in saline. The supernatant is lyophilized and stored at −80° C. Commercially available acid-resistant hypromellose capsules (DRCaps, Capsugel) are used. Double-encapsulated capsules are prepared by using a filled size 0 capsule packaged inside a size 00 capsule. Capsules are manually filled using a 24-hole filler (Capsugel) to a final concentration of about 10¹¹ cells/capsule. The capsules are stored at −80° C. in 50 mL conical tubes until needed. Once removed from the freezer, a 1 g silica gel canister (Dry Pak Industries, Encino, Calif.) is added to the container.

### Delivery

The fecal sample obtained from the donor subject can be processed, formulated, and packaged to be in an appropriate form in accordance with the delivery means in the FMT procedure, which may be by direct deposit in the recipient's lower gastrointestinal track (e.g., wet or semi-wet form) or by oral ingestion (e.g., frozen dried encapsulated). In some embodiments, the processed fecal sample can be formulated for FMT by direct transfer to the GI tract (e.g., via colonoscopy or via nasal intubation). In some embodiments, the processed fecal sample can be formulated for FMT by rectal deposit.

In some embodiments, the processed fecal sample can be stored as an aqueous solution or lyophilized powder preparation. A delivery vehicle is suitable for the route of delivery or administration. In some embodiments, the delivery vehicle is suitable for oral administration. In some embodiments, the delivery vehicle is suitable for direct transfer to the GI track. In some embodiments, the delivery vehicle further stabilizes the microbial species, and/or enhances the efficacy of the microbial species.

In some embodiments, the delivery vehicle is a buffer, such as phosphate buffered saline (PBS), Luria-Bertani Broth, phage buffer (100 mM NaCl, 100 mM Tris-HCl, 0.01% (w/v) Gelatin), or Tryptic Soy broth (TSB). In some embodiments, the delivery vehicle comprises food grade oils, and inorganic salts useful for adjusting the viscosity of the composition. Examples of pharmaceutically acceptable carriers are well known, and one skilled in the pharmaceutical art can easily select carriers suitable for particular routes of administration (Remington's Pharmaceutical Sciences, Mack Publishing Co., Easton, Pa., 1985). Suitable pharmaceutical carriers include, but are not limited to, sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. The final form may be sterile and may also be able to pass readily through an injection device such as a hollow needle. The proper viscosity may be achieved and maintained by the proper choice of solvents or excipients.

In some embodiments, the delivery vehicle comprises other agents, excipients, or stabilizers to improve properties of the composition, which do not reduce the effectiveness of the microbial species. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. Examples of emulsifying agents include tocopherol esters such as tocopheryl polyethylene glycol succinate and the like, PLURONIC®, emulsifiers based on polyoxy ethylene compounds, Span 80 and related compounds and other emulsifiers known in the art and approved for use in animals or human dosage forms. The compositions (such as pharmaceutical compositions) can be formulated so as to provide rapid, sustained or delayed release of the active ingredient after administration to an individual by employing procedures well known in the art.

In some embodiments, the processed fecal sample comprises a delivery vehicle suitable for oral administration. In some embodiments, the delivery vehicle is an aqueous medium, such as deionized water, mineral water, 5% sucrose solution, glycerol, dextran, polyethylene glycol, sorbitol, or such other formulations that maintain phage viability, and are non-toxic to animals, including lactating mammals and humans. In some embodiments, the composition is prepared by resuspending purified phage preparation in the aqueous medium.

### V. Kits and Compositions for Use in Treating Obesity or Metabolic Diseases

The present invention also provides novel kits and compositions that can be used for facilitation of patient weight loss/treating or preventing metabolic diseases or for assessing a patient's likelihood of later developing obesity and/or metabolic diseases. For example, a kit is provided that comprises a first container containing a first composition comprising an effective amount of one microbial species selected from the group consisting of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and viruses in Table 9, and a second container containing a second composition comprising an effective amount of another, different microbial species selected from the group consisting of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and viruses in Table 9. In some variations, the first and/or second composition may contain two of the bacterial or viral species of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and viruses in Table 9.

In some cases, the first and/or second composition may comprise a fecal material from a donor, which has been processed, formulated, and packaged to be in an appropriate form in accordance with the delivery means in the FMT procedure, which may be by direct deposit in the recipient's lower gastrointestinal track (e.g., wet or semi-wet form) or by oral ingestion (e.g., frozen, dried/lyophilized, encapsulated). Alternatively, the first and/or second composition may not contain any donor fecal material but is an artificially mix containing the preferred viral and/or bacterial species, such as one or more set forth in Table 2 or 9 or other viral or bacterial species named above and herein, at an appropriate ratio and quantity. The first and/or second composition may be formulated and packaged in accordance with the intended means of delivery to the patient, for example, by oral ingestion, nasal delivery, or rectal deposit.

Optionally, the second composition may be similarly formulated from donor fecal material or other non-fecal originated material for oral, nasal, or rectal delivery. Typically, the second composition contains a viral or bacterial species or a combination of viral and/or bacterial species different from that comprised in the first composition. The first and second compositions may or may not be formulated for the same delivery method or route.

The first and second compositions are typically kept separately in two different containers in the kit. In some cases, the first and second compositions may be combined in a single composition so that they can be administered to the patient together, for example, by oral or local delivery, at the same time.

Lastly, a kit is provided for the quantitative detection of one or more viral species such as the viral species set forth in Tables 1, 2, and 7-9 as well as others named herein. The kit comprises reagents for quantitative detection of each of the viral species, for example, such reagents may comprise a set of oligonucleotide primers for the amplification, such as polymerase chain reaction (PCR) especially quantitative PCR, of a polynucleotide sequence derived from, and preferably unique to, each one of the pertinent viral species (such as any one or more of the viral species set forth in Tables 1, 2, and 7-9 and others identified above and herein).

### EXAMPLES

The following examples are provided by way of illustration only and not by way of limitation. Those of skill in the art will readily recognize a variety of non-critical parameters that could be changed or modified to yield essentially the same or similar results.

### Example I

### Cross-Sectional Study on Obesity and Type 2 Diabetes in Hong Kong Study Cohort

A total of 131 subjects were recruited in Hong Kong. Subjects were grouped into three groups: (1) Lean, healthy control (BMI<23 Kg/m²) without type 2 diabetes, n=68; (2) Ob, obese (BMI≥28 Kg/m2) without type 2 diabetes, n=10; and (3) ObT2, obese (BMI≥28 Kg/m2) with type 2 diabetes, n=53. All subjects consented to donate fecal sample and to the questionnaire investigation, where written informed consents were obtained. Fecal samples from the study subjects were stored at −80° C. for downstream microbial analyses. The study was approved by The Joint Chinese University of Hong Kong, New Territories East Cluster Clinical Research Ethics Committee (The Joint CUHK-NTEC CREC, CREC Ref. No: 2016.407).

### Fecal Viral DNA Extraction and Sequencing

VLPs were enriched by using a protocol according to previously described methods. Approximately 200 mg of stool was suspended in 400 μl saline-magnesium buffer (0.1M NaCl, 0.008 M MgSO₄·7H₂O, 0.002% gelatin, 0.05 M Tris pH7.5) by vortexing for 10 min. Stool suspensions were then cleared by centrifugation at 2,000×g to remove debris and cells. Clarified suspensions were passed through one 0.45 μm followed by 0.22 μm filters to remove residual host and bacterial cells. Samples were treated with lysozyme (1 mg/ml at 37° C. for 30 min) followed by chloroform (0.2× volume at RT for 10 min) to degrade any remaining bacterial and host cell membranes. Non-virus protected DNA was degraded by treatment with 1 U Baseline zero DNase (Epicenter)) followed by heat inactivation of DNases at 65° C. for 10 min. VLPs were lysed (4% SDS plus 38 mg/ml Proteinase K at 56° C. for 20 min), treated with CTAB (2.5% CTAB plus 0.5 M NaCl at 65° C. for 10 min), and nucleic acid was extracted with Phenol:Chloroform:Isoamyl Alcohol pH 8.0 (Sigma). The aqueous fraction was washed once with an equal volume of chloroform, purified and concentrated on a column (DNA Clean & Concentrator TM 89-5, Zymo Research). VLP DNA was amplified for 1.5-2 h using Phi29 polymerase (GenomiPhi V2 kit, GE Healthcare) prior to sequencing. DNA libraries were constructed through the processes of end repairing, purification, and PCR amplification. After DNA libraries construction, DNA libraries were sequenced by Illumina Novaseq 6000 with paired-end 150 bp sequencing strategy by Novogene, Beijing, China.

### Quality Trimming of Raw Sequences

Shot-gun metagenomics reads were quality-filtered and dehost contamination were done by KneadData (v0.7.2). Java8 (v1.8.0_152-release), Bowtie2 (v2.3.4.3) and Trimmomatic (v0.39.1) were preinstalled to support KneadData running. We trimmed any leading or trailing N-bases and other bases that had Phred quality scores of 3 or below, cut each sequence read with a 4-base sliding window trimmer that required minimum average quality scores of 15, and removed any sequence reads that had 50 bases or fewer. We then cut adapter sequences in paired-end reads by checking for maximum mismatch count, simple and palindromic matches of 2, 10 and 30 bases, respectively, with a library of universal Illumina TruSeq3-PE-2.fa adapter sequences. Then the post—quality-trimmed metagenomic reads were pass to Bowtie2 for decontaminated host contaminations. We performing end-to-end Bowtie-2 alignment with “very-sensitive” preset options against an indexed database human genome (hg38), the reads without aliment to human genome were keep as clean reads.

### Virus Taxonomy Annotation

We assembly paired end VLPs reads into contigs by Megahit (v1.0.3) with default parameter. We only keep contigs with length larger than 1,000 bp and clustered the contigs at a 95% identity level using CD-HIT (v4.7) to generate a unique contig consortium. Open Reading Frame (ORF) were predicted and extracted from contigs using the Glimmer3 (v 3.02) and a minimum length threshold of 100 amino acids. The translated amino acid sequences of predicted ORFs from the VLP contigs were matched against the standard subset of the standalone entire UniProt TrEMBL database as of Feb. 11, 2019, that contained only virus and phage reference proteins, using blastx (e<10-5) provided by Diamond (v 0.9.24). Each contig was assigned taxonomy based on the most abundant taxa contained within that contig using a voting system as described previously for virus taxonomic assignment at different taxon levels. The voting system first annotated each ORF of a contig of interest with the best-hit virus taxonomy. It then compared all of the taxonomic assignments of the ORFs within the contig of interest, and annotated the contig with the majority ORF assignment. Contigs with less than one ORF per 10 kb were not assigned taxonomy as this suggests a contig of only limited similarity. Contigs without a majority ORF taxonomic assignment due to ties of multiple major taxa were assigned as having multiple possible taxonomic annotations. Because some contigs shared the same taxonomic identities, the contig table was collapsed by taxonomic identity, meaning the contig relative abundances were summed if they shared identity. In parallel, we blast the contigs to NCBI Refseq genome reads and remove any contig assign to cellular organisms. We then aligned the whole DNA sequencing reads (removed bacterial, fungal and archaea reads) to the unique contig consortium by Bowtie2 to get read counts table for each sample. The mapped read counts, contig lengths and total read counts were used to normalize the original read counts to RPKM (Reads Per Kilobase Million) and exported for downstream analysis.

### Statistical Methods

The virome and bacteriome abundance table were imported into R (v3.6.1). Alpha diversity was calculated with R package phyloseq (v1.28.0). Data process and visualization were performed by R packages (tidyverse v1.2.1, pheatmap v 1.0.12 and ggsignif v0.6.0). Two-tailed Wilcoxon Rank Sum test and Kruskal-Wallis test was used to determine statistically significant difference between groups. MaAsLin2 (multivariate association with linear models) was used to identify associations between clinical metadata and viral abundance while controlling for confounders, namely age, gender, alcohol and smoking. The viral-type of each fecal sample was analyzed with the partition around medoids (PAM) method using the relative abundance of viruses in each community.

Reduction of Virome Richness and Diversity in Obese Subjects with Type 2 Diabetes

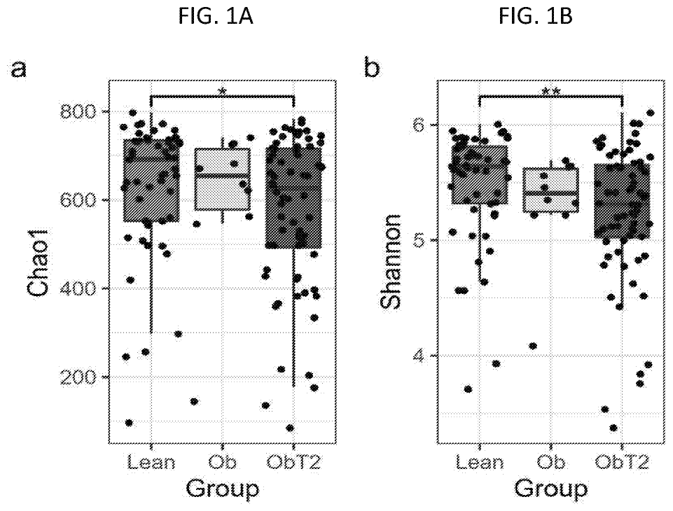

We found a significant reduction in viral richness (Chao1) and diversity (Shannon) for obese subjects with type 2 diabetes mellitus compared with lean subjects (Wilcoxon, p=0.045 and p=0.0052, respectively, FIGS. 1A and 1B). This result suggests a higher alpha diversity of viral microbiota might be beneficial for body weight reduction and type 2 diabetes control.

Viral Species Enriched or Depleted in Obese Subjects with Type 2 Diabetes

We next correlated the viral species abundance profile with the disease phenotype via MaAsLin2 to define the gut viral signatures which were associated with obesity and T2DM (type 2 diabetes mellitus). After correction for confounders (age, gender, alcohol and smoking), 11 viral species were identified to be significantly different in obese with T2DM compared to lean control subjects. Four of the 11 species (Bacteroides phage, Pectobacterium phage, Achromobacter phage, and Azobacteroides phage) were enriched (Table 1), whereas 7 species (Diachasmimorpha longicaudata entomopoxvirus, Megavirus, Oenococcus phage, Saudi moumouvirus, Clostridium botulinum C phage, Emiliania huxleyi virus, and Lausanne virus) were depleted in obesity with T2DM compared with lean control (Table 2).

TABLE 1
Viral species enriched in obese subjects with type 2 diabetes

| Viral Species | NCBI:txid* |
|---|---|
| Bacteroides phage | 2486354, 2486353, 2306278, 2303977, |
| | 2301731 |
| Pectobacterium phage | 2686475, 2662284, 2662283, 2652428, |
| | 2652426, 2608323, 2608319, 2608298, |
| | 2500578, 2500577, 2500576, 2489635, |
| | 2489634, 2489628, 2489627, 2489626, |
| | 2489618, 2489617, 2488835, 2320198 |
| Achromobacter phage | 2591403, 1610509, 1589748, 1589746, |
| | 1416009, 1416008 |
| Azobacteroides phage | 1920526 |

*Including but not limited to the listed txid for the strains.

TABLE 2
Viral species depleted in obese subjects with type 2 diabetes

| Viral Species | NCBI:txid* |
|---|---|
| Diachasmimorpha | 109981 |
| longicaudata | |
| entomopoxvirus | |
| Megavirus | 2109586, 1686770, 1686597, 1686596, |
| | 1686595, 1686594, 1643508, 1643305, |
| | 1643304, 1643303, 1643301, 1643300, |
| | 1643298, 1643296, 1242815, 1242814, |
| | 1235314, 1128143, 1128142, 1128141 |
| Oenococcus phage | 2201414, 1885654, 1885653, 1885652, |
| | 1885651, 1885650, 1885649, 1885648, |
| | 1885647, 1885646, 1885645, 1885644, |
| | 1885643, 1885642, 1885641, 1885640, |
| | 1435411, 1432848, 1432847, 264987 |
| Saudi moumouvirus | 1956188 |
| Clostridium | 12336 |
| botulinum C phage | |
| Emiliania huxleyi virus | 181208 |
| Lausannevirus | 999883 |

*Including but not limited to the listed txid for the strains.

Subjects with Obesity and Type 2 Diabetes Differ in Viral Types

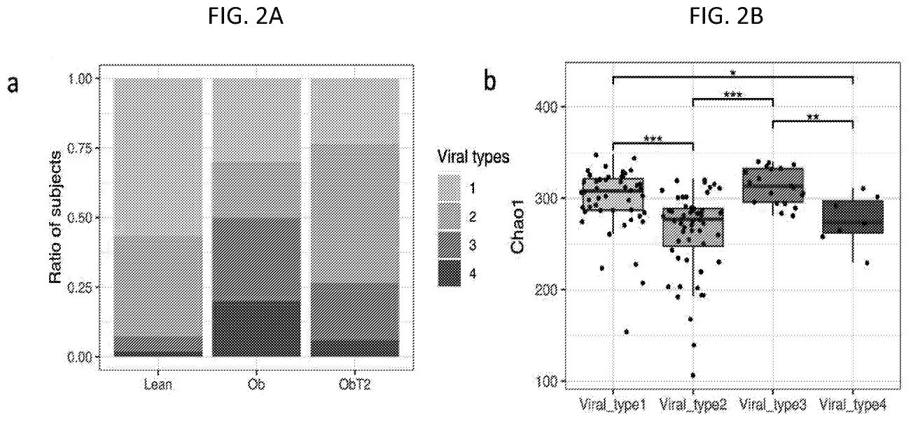

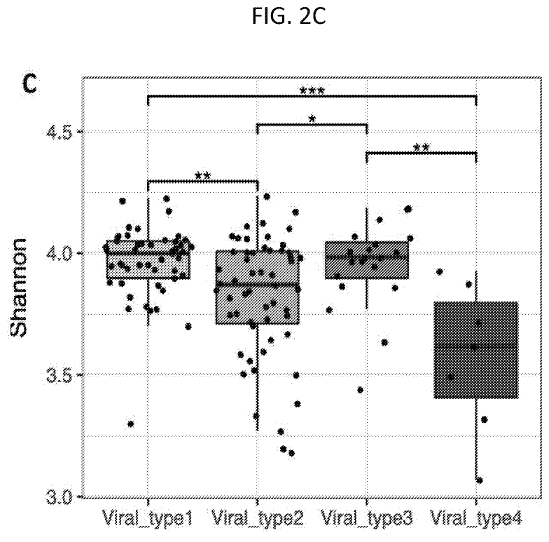

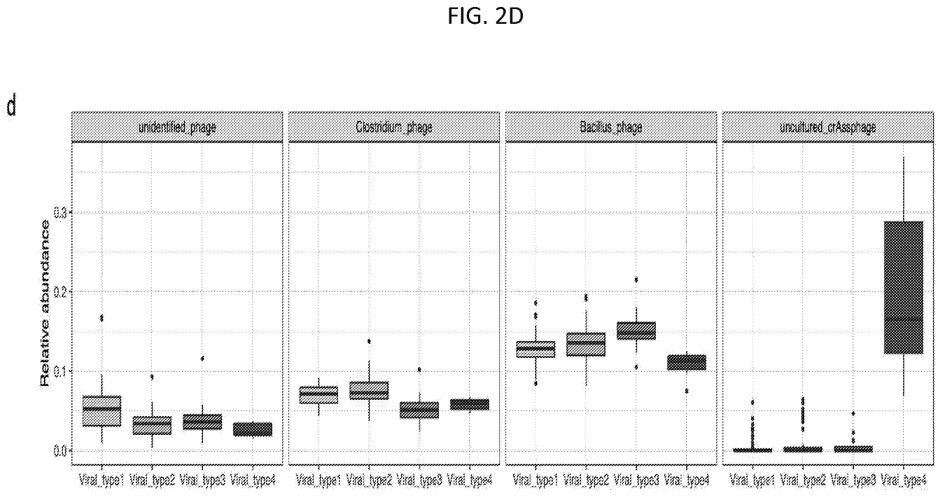

We calculated viral-types to explore difference in gut viral community between subjects. We obtained 4 viral-types for all subjects based on the silhouette index. We found that the proportion of subjects belonging to the Ob (obese), ObT2 (Obese and type 2 diabetes mellitus) and lean groups varied in viral-types (Fisher's exact test, p<0.001, FIG. 2A). Particularly, only 1 subject in viral type 4 belong to lean control group and the other 6 subjects were obese with or without T2D. Among the four viral-types, richness and shannon diversity were significantly reduced in viral-type 2 and viral-type 4 compared with the other two viral-types (FIGS. 2B and 2C). A significant increased abundance of uncultured crAssphage was observed in the viral-type 4 which mainly comprised of Ob and ObT2 subjects (FIG. 2D). This result suggests the obese and T2DM related gut virome alteration correlated with viral-type 4 compositions which is associated with a decrease in viral alpha diversity indices and increase unculture crAssphage.

Gut Virome a diversity Indices Correlate with Blood Parameters in Humans

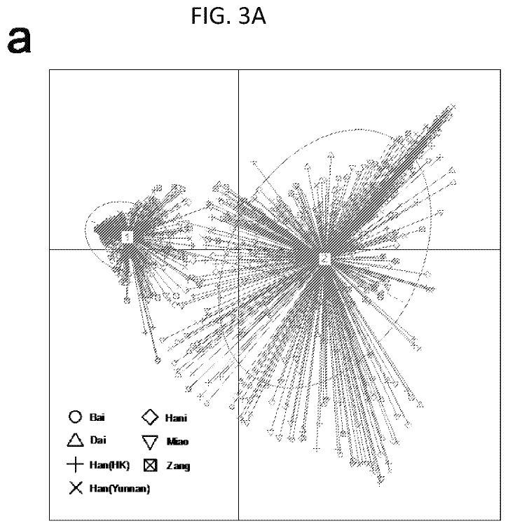

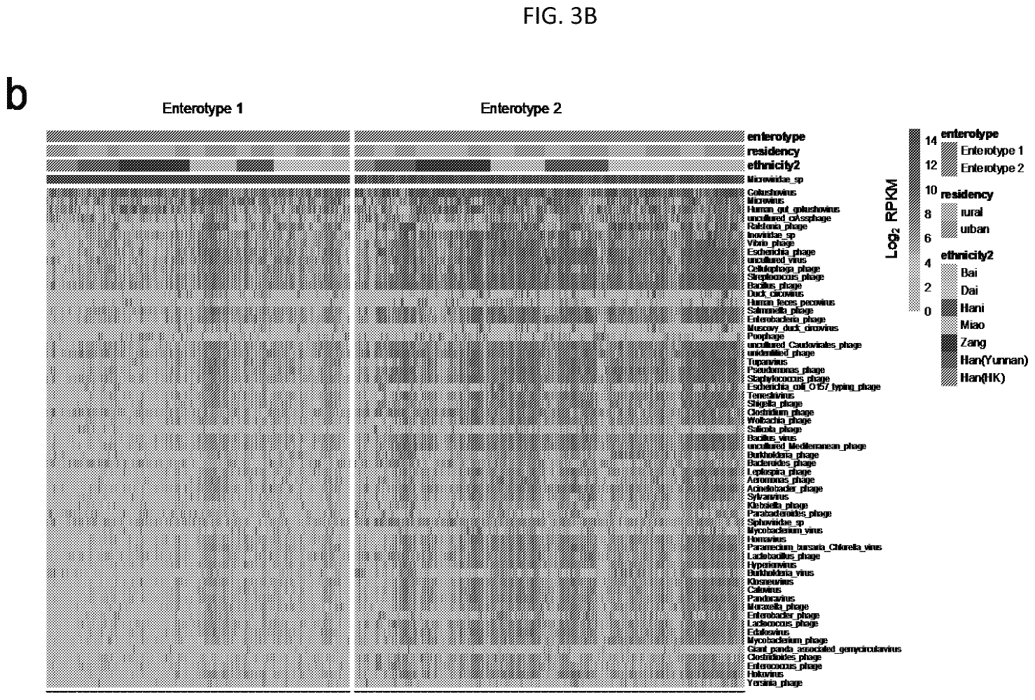

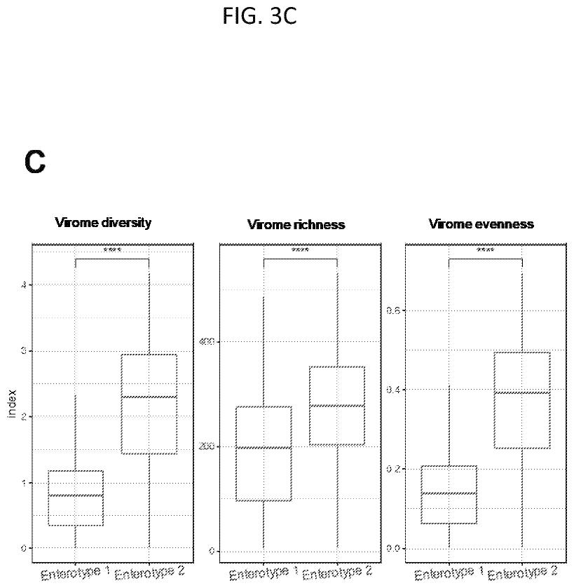

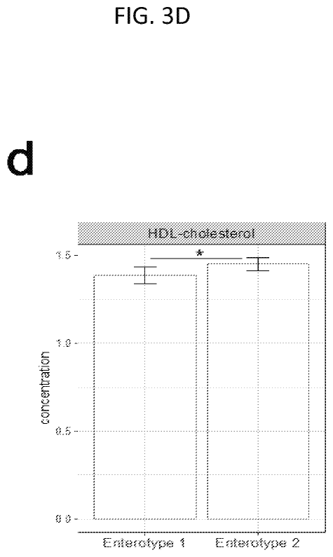

By applying partition around medoids (PAM) clustering algorithm on the virome composition profiles, all healthy subjects' viral communities converged into two clusters (referred to as gut virome enterotypes hereafter, FIG. 3A). Compared to virome enterotype1, enterotype2 was enriched in various viruses (Table 3 and FIG. 3B), leading to markedly higher virome diversity, richness and evenness for enterotype2 virome than enterotype1 virome (FIG. 3C). Subjects with virome enterotype2 showed significantly higher High-density lipoprotein cholesterol (HDL-Cholesterol, FIG. 3D), suggesting that gut enterotype2 virome (high virome a diversity and richness) may be protective against metabolic diseases associated with high blood cholesterol levels.

TABLE 3
Viral species highly present in virome enterotype2

| | Cut-off value | |
|---|---|---|
| | (Median abundance | |
| Species | (RPKM)) | NCBI:txid |
| Gokushovirus | 2904.145 | 2507516 |
| Microvirus | 236.485 | 10842 |
| Human_gut_gokushovirus | 213.9415 | 1986031 |
| uncultured_crAssphage | 61.16615 | 1211417 |
| Ralstonia_phage | 52.8797 | 247080 |
| Inoviridae_sp | 116.124 | 2219103 |
| Escherichia_phage | 283.313 | 2663325 |
| Cellulophaga_phage | 134.1725 | 1327992 |
| Streptococcus_phage | 277.984 | 2607969 |
| Bacillus_phage | 402.7845 | 2663324 |
| Salmonella_phage | 147.3585 | 1813783 |
| Enterobacteria_phage | 116.7785 | 115985 |
| Tupanvirus | 54.13305 | 2126984 |
| Pseudomonas_phage | 97.05995 | 2679904 |
| Staphylococcus_phage | 120.7475 | 2681608 |
| Terrestrivirus | 36.93835 | 2487775 |
| Shigella_phage | 38.9355 | 55884 |
| Clostridium_phage | 70.3971 | 1162306 |
| Wolbachia_phage | 28.7505 | 112596 |
| Bacillus_virus | 61.0145 | 1406782 |
| uncultured_Mediterranean_ | 55.27245 | 1868660 |
| phage | | |
| Leptospira_phage | 15.6752 | 1334243 |
| Aeromonas_phage | 12.11985 | 2653964 |
| Acinetobacter_phage | 36.4598 | 2690230 |
| Sylvanvirus | 16.3076 | 2487774 |
| Klebsiella_phage | 8.388675 | 2681196 |
| Homavirus | 22.1253 | 2487769 |
| Paramecium_bursaria_ | 26.10795 | 240265 |
| Chlorella_virus | | |
| Lactobacillus_phage | 35.62705 | 2510944 |
| Hyperionvirus | 22.0782 | 2487770 |
| Klosneuvirus | 24.65845 | 1977640 |
| Catovirus | 17.37155 | 1977631 |
| Pandoravirus | 21.128 | 2107707 |
| Moraxella_phage | 16.1367 | 1647532 |
| Lactococcus_phage | 25.0317 | 1262538 |
| Edafosvirus | 23.47485 | 2487765 |
| Mycobacterium_phage | 23.94215 | 1506716 |
| Clostridioides_phage | 13.95605 | 2069614 |
| Enterobacter_phage | 1.83769 | 1455074 |
| Enterococcus_phage | 15.7872 | 673832 |
| Burkholderia_phage | 18.25635 | 279280 |
| Bacteroides_phage | 30.91055 | 2301731 |
| Hokovirus | 17.22985 | 1977638 |
| Siphoviridae_sp | 34.82875 | 2170413 |
| Escherichia_coli_O157_ | 1.904395 | 1508671 |
| typing_phage | | |
| Yersinia_phage | 1.038575 | 1195074 |
| Parabacteroides_phage | 2.04224 | 1655644 |
| Burkholderia_virus | 5.64205 | 335797 |
| Mycobacterium_virus | 0.8171955 | 1194642 |
| Human_feces_pecovirus | 0.201434 | 1820160 |
| Poophage | 0.760416 | 1926504 |
| Salicola_phage | 0.8040775 | 754067 |
| Duck_circovirus | 0.1444295 | 324685 |
| Muscovy_duck_circovirus | 0.06655395 | 257468 |

### Correlation Between Gut Fungi and Blood Parameters

We clinically profiled the blood biochemical parameters and correlated them with the gut mycobiome profile. Among all significant fungus-blood parameter correlations, Candida dubliniensis exhibited the strongest inverse correlation with blood glucose. Furthermore, we also found that Candida dubliniensis showed a positive correlation with high-density lipoprotein cholesterol (HDL-C) and an inverse correlation with low-density lipoprotein cholesterol (LDL-C). This data suggests that Candida dubliniensis may have a role associated with protection against metabolic diseases.

### Example II. Gut Virome and Mycobiome Across Six Ethnicities in Urban and Rural China

### Cohort Description and Study Subjects

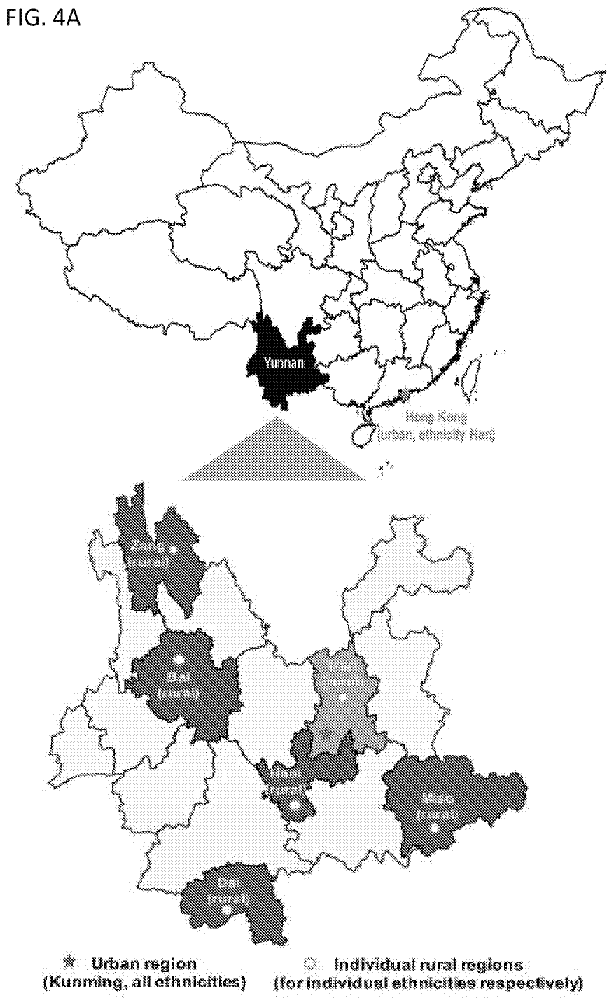

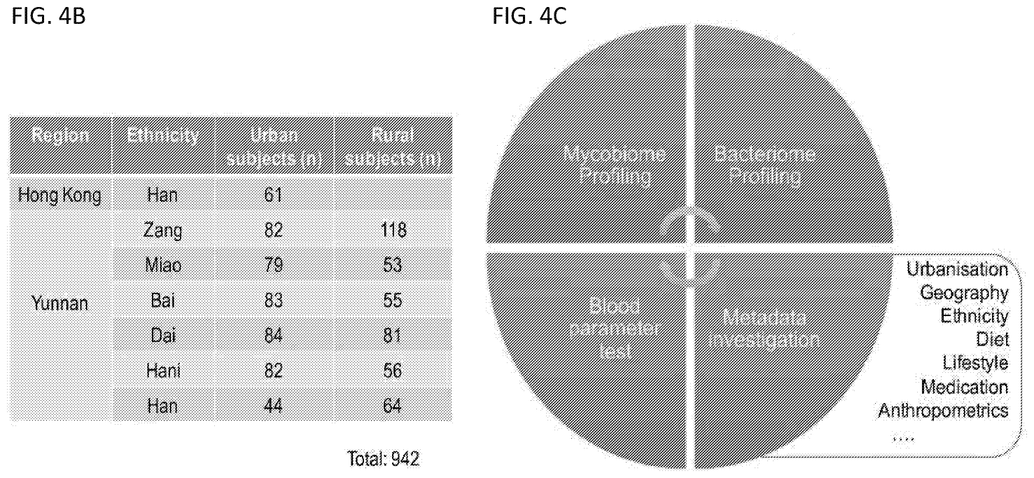

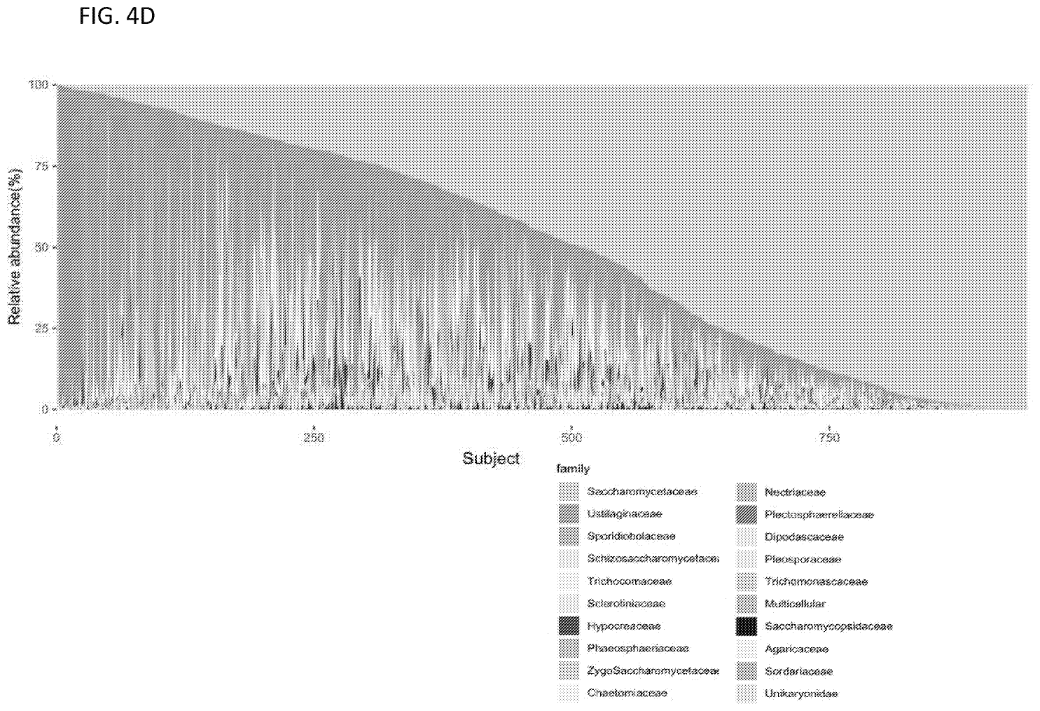

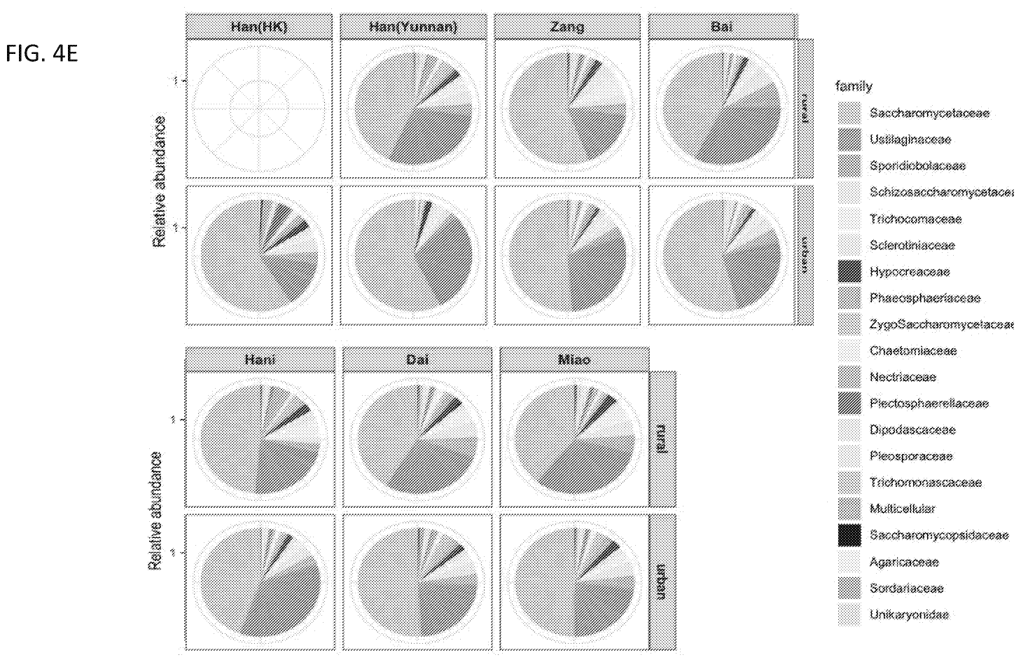

A total of 942 healthy Chinese from Hong Kong (n=61, all ethnically Han and urban residents) and Yunnan province (n=881, subjects were enrolled from ethnicities Han, Zang, Miao, Bai, Dai, and Hani; rural and urban residents included for each ethnic group) were recruited (FIGS. 4A and 4B, Table 4). The study was approved by The Joint Chinese University of Hong Kong, New Territories East Cluster Clinical Research Ethics Committee (The Joint CUHK-NTEC CREC, CREC Ref. No: 2016.407) and by the Institutional Review Board (IRB) and Research Ethics Committee of the First Affiliated Hospital of Kunming Medical School (Ref. No: 2017. L.14). An additional obese cohort (49 obese subjects, body mass index, BMI≥28.0 kg/m²; 49 lean subjects, BMI 18.5-22.9 kg/m²) was included. All subjects consented to providing fecal samples, and completed environmental and dietary questionnaires. Written informed consents were obtained from all subjects. Fecal samples from the study subjects were stored at −80° C. for mycobiome and bacterial microbiome (bacteriome) analyses. Clinical data were obtained by medical practitioners. Dietary questionnaire investigation was conducted by a dedicated dietitian. Dietary questionnaire was designed for Chinese populations, consisting of conventional Chinese foods, ranging from staple foods, side dishes (various types of cooked meats and vegetables), fruits, beverages (Chinese/herbal tea, coffee), and ethnic minority foods in Yunnan (insects, flowers and various types of mushrooms). Intake of these food categories in the recent 3 months was documented as binary. A majority of the study subjects also consented to blood tests for blood glucose and fasting cholesterol measurements.

TABLE 4

| | | | | | | | | | Population |
|---|---|---|---|---|---|---|---|---|---|
| | | | | | | | | GDP/ | density |
| | | | | latitude | | Area | | population | (People/ |
| Sampling | Inhabiting | rural/ | altitude | and | Population | size(Square | GDP(million, | (RMB/ | square |
| region | ethnicity | urban | (meter) | longtitude | size | kilometer) | RMB) | person) | kilometer) |
| Hong Kong | Han | urban | 1.3 | 22°12′5 | 7483000 | 1107 | 2845.3 | 380235.20 | 6759.7 |
| | | | | 4.0″N | | | | | |
| | | | | 113°54′ | | | | | |
| | | | | 2.05″E | | | | | |
| Kunming | Han, | urban | 1890 | 25°02′2 | 6432000 | 21473 | 520.7 | 80954.60 | 299.5 |
| (Yunnan) | Zang, | | | 9.9″N | | | | | |
| | Miao, Bai, | | | 102°41′ | | | | | |
| | Hani, Dai | | | 35.7″E | | | | | |
| Village Duoyijia | Han | rural | 1710 | 25°27′2 | 149000 | 993 | 6.5 | 43624.16 | 150.1 |
| (Fumin, Yunnan) | | | | 9.4″N | | | | | |
| | | | | 102°41′ | | | | | |
| | | | | 24.5″E | | | | | |
| Town Jiantang | Zang | rural | 3290 | 27°49′0 | 154100 | 11613 | 11.1 | 72031.15 | 13.3 |
| (Shangri-La, | | | | 1.4″N | | | | | |
| Yunnan) | | | | 99°42′1 | | | | | |
| | | | | 5.3″E | | | | | |
| Town Fengyu | Bai | rural | 2220 | 25°59′2 | 275800 | 2875 | 5.9 | 21392.31 | 95.9 |
| (Eryuan, Dali, | | | | 6.5″N | | | | | |
| Yunnan) | | | | 99°55′4 | | | | | |
| | | | | 0.3″E | | | | | |
| Town Manlai | Hani | rural | 680 | 23°39′3 | 200000 | 2858 | 7.2 | 36000.00 | 70.0 |
| (Yuxi, | | | | 3.6″N | | | | | |
| Yuanjiang, | | | | 101°51′ | | | | | |
| Yunnan) | | | | 40.9″E | | | | | |
| Dai Autonomous | Dai | rural | 540 | 21°50′5 | 440000 | 6958 | 19.2 | 43636.36 | 63.2 |
| district | | | | 8.9″N | | | | | |
| (Xishuangbanna, | | | | 100°55′ | | | | | |
| Yunnan) | | | | 37.8″E | | | | | |
| Village Heishan | Miao | rural | 1330 | 23°33′2 | 109600 | 3064 | 20.8 | 189781.02 | 35.8 |
| (Wenshan, | | | | 3.7″N | | | | | |
| Yunnan) | | | | 103°36′ | | | | | |
| | | | | 06.5″E | | | | | |

Stool sample collection followed a standardized operation procedure (SOP) for all sites. Samples from urban areas were stored within 1 hour of collection at −80° C. freezer and those collected from rural areas were immediately stored on dry ice and transported to the laboratory within 8 hours in one batch, followed by storage in −80° C. freezer. All stool samples from Hong Kong and Yunnan were finally transported to the Center for Microbiome Research (Shenzhen Research Institute, The Chinese University of Hong Kong, Shenzhen, China), where DNA extraction was extracted by four trained laboratory staff simultaneously. PERMANOVA (adonis) showed no significant influence of sample processing by different staff on mycobiome variations.

### Fungi-Enriched Fecal DNA Extraction and DNA Sequencing

Fecal DNA was extracted using Maxwell® RSC PureFood GMO and Authentication Kit (Promega) with modifications to increase the yield of fungal DNA. Approximately 100 mg from each stool sample was prewashed with 1 ml ddH₂O and pelleted by centrifugation at 13,000×g for 1 min. The pellet was resuspended in 800 μL TE buffer (pH 7.5), supplemented with 1.6 μl 2-mercaptoethanol and 500 U lyticase (Sigma) digesting cell walls of fungi, and incubated at 37° C. for 60 min, which increase the lysis efficacy of fungal cell. The sample was then centrifuged at 13,000×g for 2 min and the supernatant was discarded. After this pretreatment, DNA was subsequently extracted from the pellet using a Maxwell® RSC PureFood GMO and Authentication Kit (Promega) following manufacturer's instructions. Briefly, 1 ml of CTAB buffer was added to the pellet and vortexed for 30 s, then the solution heated at 95° C. for 5 min. After that, samples were vortexed thoroughly with beads (Biospec, 0.5 mm for fungi and 0.1 mm for bacteria, 1:1) at maximum speed for 15 min. Following this, 40 μl proteinase K and 20 μl RNase A were added and the mixture Incubated at 70° C. for 10 min. The supernatant was then obtained by centrifuging at 13,000×g for 5 min and placed in a Maxwell® RSC instrument for DNA extraction. The extracted fecal DNA was used for ultra-deep metagenomics sequencing via Ilumina Novoseq 6000 (Novogen, Beijing, China). An average of 52±6.3 million reads (12G clean data) per sample were obtained.

### Quality Filtering Metagenome Sequence Data

Raw sequence reads were filtered and quality-trimmed using Trimmomatic v0.36 25 as follows: 1) Trimming low quality base (quality score <20); 2) Removing reads shorter than 50 bp; 3) removing sequences less than 50 bp long; 4) Tracing and cutting off sequencing adapters. Contaminating human reads were filtering using Kneaddata (Reference database: GRCh38 p12) with default parameters. Accession codes: Sequence data have been deposited to the NCBI Sequence Read Archive under BioProject accession number PRJNA588513.

### Profiling the Bacterial and Fungi Microbiome

Profiling of bacterial microbiome (bacteriome) was performed via MetaPhlAn2 by mapping reads to clade-specific markers26 and annotation of species pangenomes through Bowtie2 27. Profiling of mycobiome was performed via HumanMycobiomeScan.

Clinical Metadata and Covariation with Mycobiome

We collected subject clinical metadata including anthropometric features, ethnicity information, geography, rural versus urban residency, medication history, dietary habit, lifestyle, and bowel habits. All metadata variables were classified into the following 6 categories: urbanization (rural/urban residency, bath/shower frequency, duration of time residing in urban cities, education level, travel frequency, stress at work, convenience food consumption), geography (Hong Kong versus Yunnan residency), ethnicity (six ethnic groups), medication (western medicine, Chinese medicine, prebiotics/probiotics, antibiotics), dietary habit (frequency of intake of fiber-rich vegetables, meat, and wild foods), and general metadata (age, gender anthropometric parameters, breastfeeding, bowel habit, stool consistency, animal contact). Covariates of mycobiome variation were identified by calculating the association between continuous or categorical phenotypes and species-level community ordination with envfit function in the vegan R package (999 permutations; false discovery rate30 FDR<5%). This function performs manova and linear correlations for categorical and continuous variables, respectively. Their combined effect size when pooled into the broader predefined categories was estimated with the bioenv function 31 in the same package, which selects the combination of covariates with strongest correlation to mycobiome variation (correlation between Gower distances of covariates and mycrobiome Bray-Curtis dissimilarity). To identify significant food-covariate associations, pairwise chi-square test with Crammer's V estimation and multiple-comparison adjustment (FDR) were performed. MaAsLin2 R package were used to identify food-fungi correlations with 5% significance level (after multiple testing correction). Distance-based Redundancy Analysis (db-RDA) analysis was performed in R to delineate the effect of urbanisation on gut mycobiome configuration across different ethnic groups.

### Microbiome Bioinformatic Analyses

Relative abundance compositional data for gut fungi and bacteria were imported into R v3.5.1. Alpha diversity metrics (Simpson and Shannon diversity, Chao1 richness) were calculated using the phyloseq package (v1.26.0). Centered log-ratio (CLR) transformation was applied to the microbiome relative-abundance compositional data. Given an observation vector of D taxa in a sample, x=[x1, x2, . . . xD], the clr transformation for the sample was obtained as follows:

$${X}_{clr} = [ log ( x 1 / G ( x ) ) , log ( x 2 / G ( x ) ) … log ( xD / G ( x ) ) ] ,$$

$$G ( x ) = \sqrt[D]{x 1 \cdot x 2 … xD} ,$$

G (x) is the geometric mean of x. Beta diversity analysis and Principal Component Analysis (PCA) were performed based on Aitchison distance of the microbial community composition. Heatmaps were generated using the pheatmap package (v1.0.10). Pearson (or Spearman) correlations and P values were calculated using cor and cor.test functions in R and visualized using the ggplot2. Correlations between microbial taxa were calculated via SpeciEasi based on inverse covariance selection method glasso, assuming a sparse data matrix, and the ϕ and ρ metrics. Inter-taxa correlation networked was viewed by Cytoscape v3.7.1.

### LEfSE Linear Discriminant Analysis and MaAsLin2 Analysis

To compare differences in the configuration of gut mycobiomes between Hong Kong and Yunnan subjects as well as the configuration of gut mycobiomes between rural and urban subjects in Yunnan, LefSE analyses were performed on the Huttenhower lab Galaxy server. MaAsLin2 analysis was performed on the mycobiome compositions to identify ethnicity-specific fungal taxa.

### Gut Mycobiome Variations

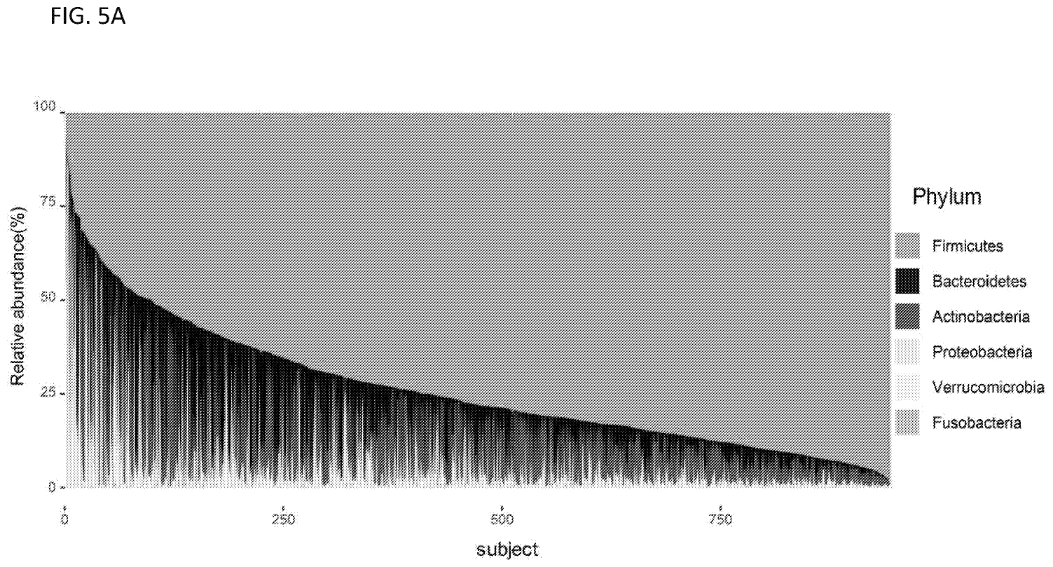

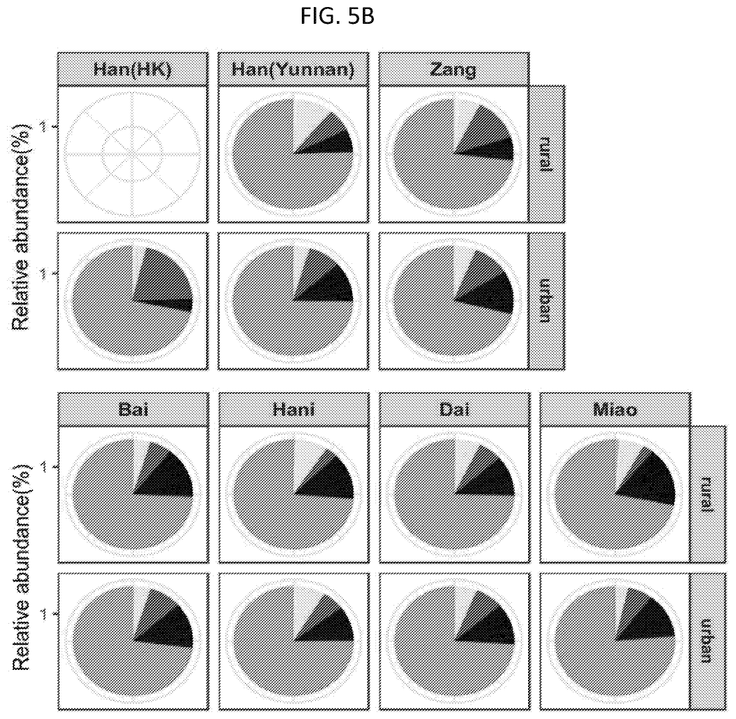

The overall gut mycobiome composition formed a continuum across all profiled individuals, and was predominated by the families Saccharomycetaceae and Ustilaginaceae (FIG. 4D). The gut mycobiome of the Hong Kong population was significantly different from that of the Yunnan populations (permutational multivariate analysis of variance [PERMANOVA], p<0.001) and was characterized by an expansion of Saccharomycetaceae and a lack of Ustilaginaceae (LDA effect sizes 4.79 and −4.84, with FDR adjusted p values 0.0195 and 0.0002, respectively, FIG. 4E). Likewise, the gut bacterial microbiome were heterogenous across populations (FIGS. 5A and 5B). The Hong Kong population harboured a higher relative abundance of the phylum Actinobacteria compared to the Yunnan populatiosn (LefSE analysis with FDR adjusted p<0.001). These results indicate that the human gut mycobiome, akin to the bacterial microbiome, is highly variable across populations.

### Identifying Core Mycobiome Covariates

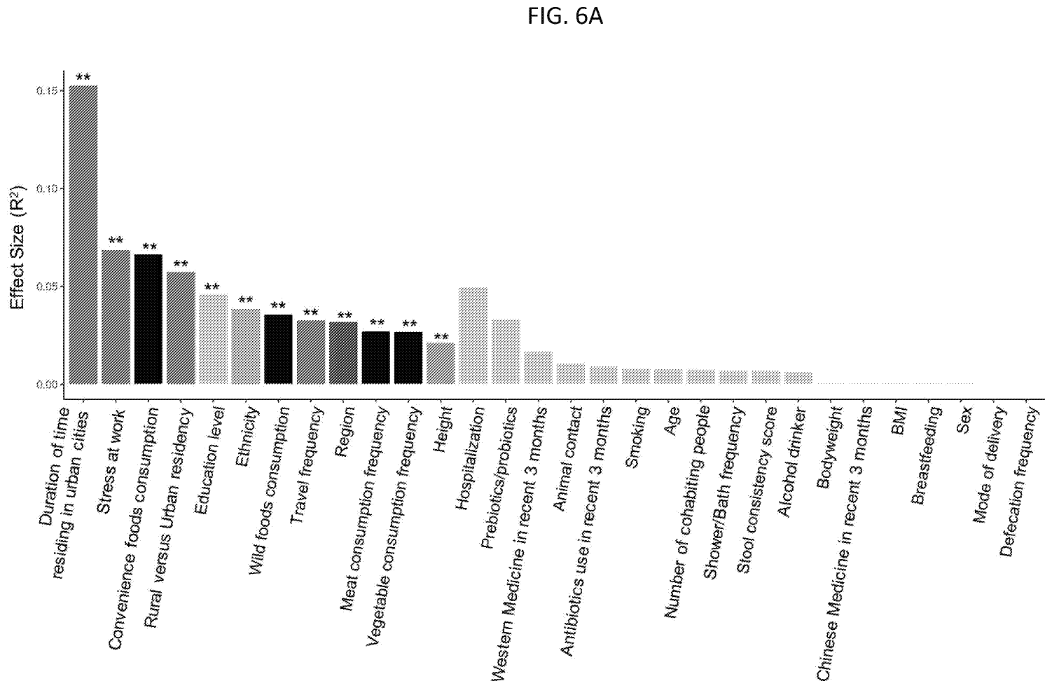

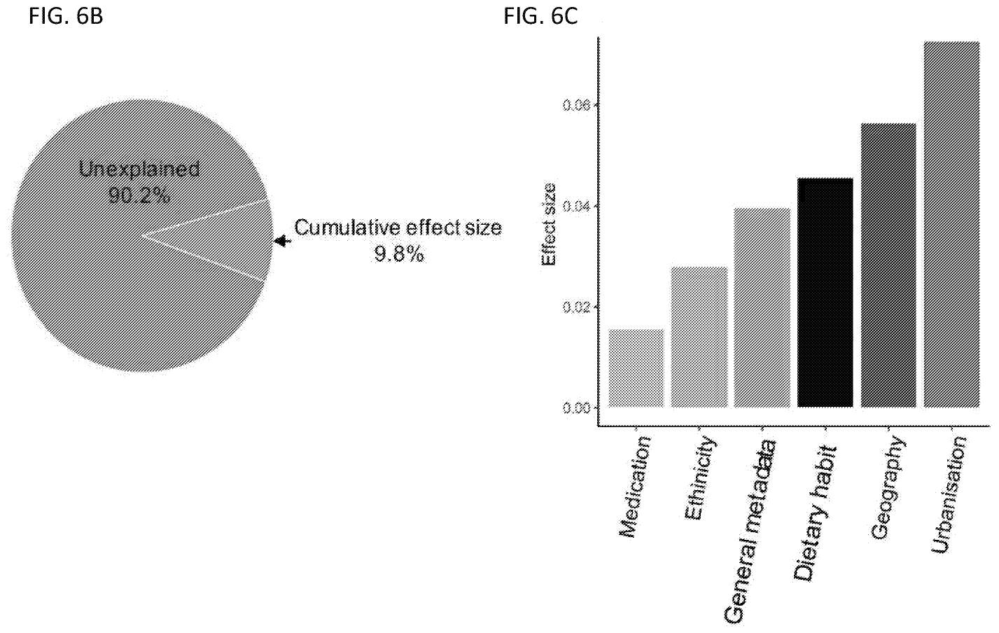

Based on subject phenotyping, we tested 33 metadata variables to identify gut mycobiome covariates. A total of 12 factors were found to correlate significantly (false discovery rate (FDR)<5%) with the overall mycobiome community variation (FIG. 6A). Among them, urbanization-related factors, including duration of time residing in urban city and stress at work, were the top two covariates of gut mycobiome. We then grouped all metadata variables to 6 predefined categories (urbanization, geography, ethnicity, dietary habit, medication, and general metadata, see Methods) and assessed the combined effect size of each category. Urbanization had the largest explanatory power on mycobiome composition, accounting for 7.3% of mycobiome variation (FIG. 6C). Geography, dietary habit, ethnicity and medication followed with decreasing combined correlation with mycobiome variation. After removing co-linear variables, these metadata variables combined explained 9.8% of species abundance variations (FIG. 6B), suggesting additional contribution from unknown factors, stochastic effects, and/or biotic interactions9. A small proportion of the studied subjects had a medication history in recent 3 months (2.1% for western drugs, 4.8% for Chinese medicine, 3.1% for prebiotic or probiotic supplements, and 1.3% for antibiotics exposure), giving rise to insignificant effects for each of these medication factors and a modest combined effect size of 1.6% for medication on population mycobiome variation (FIGS. 6A and 6C). In addition, stool consistency score, defecation frequency, mode of delivery and breastfeeding, all common bacterial microbiome covariates, did not show significant effects on mycobiome variation (FIG. 6A).

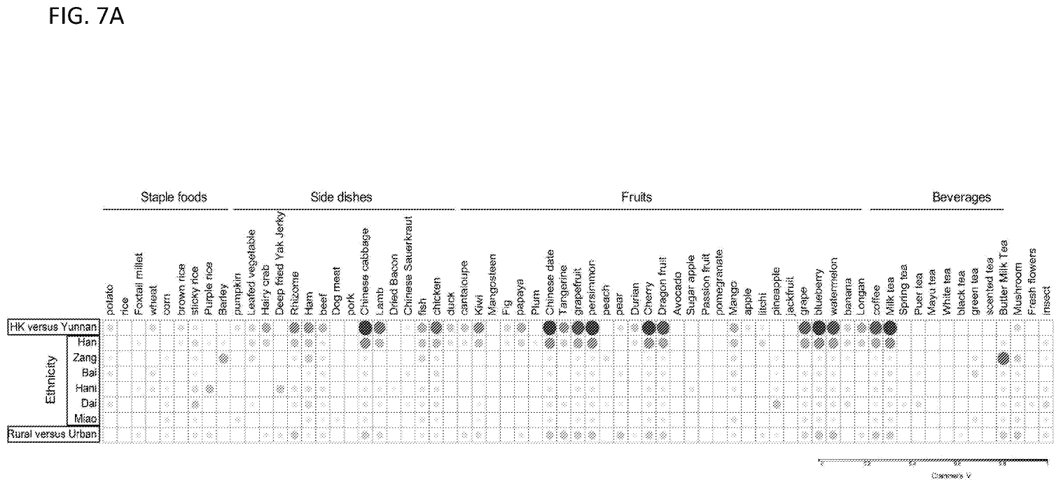

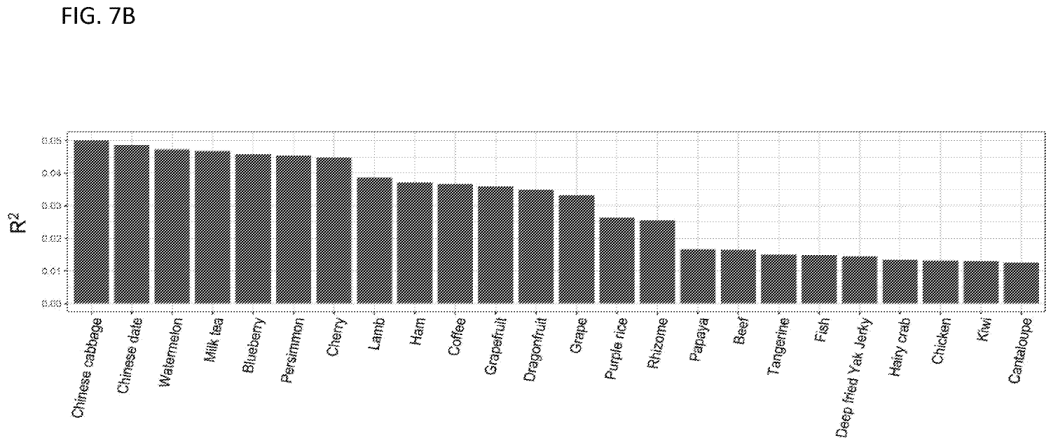

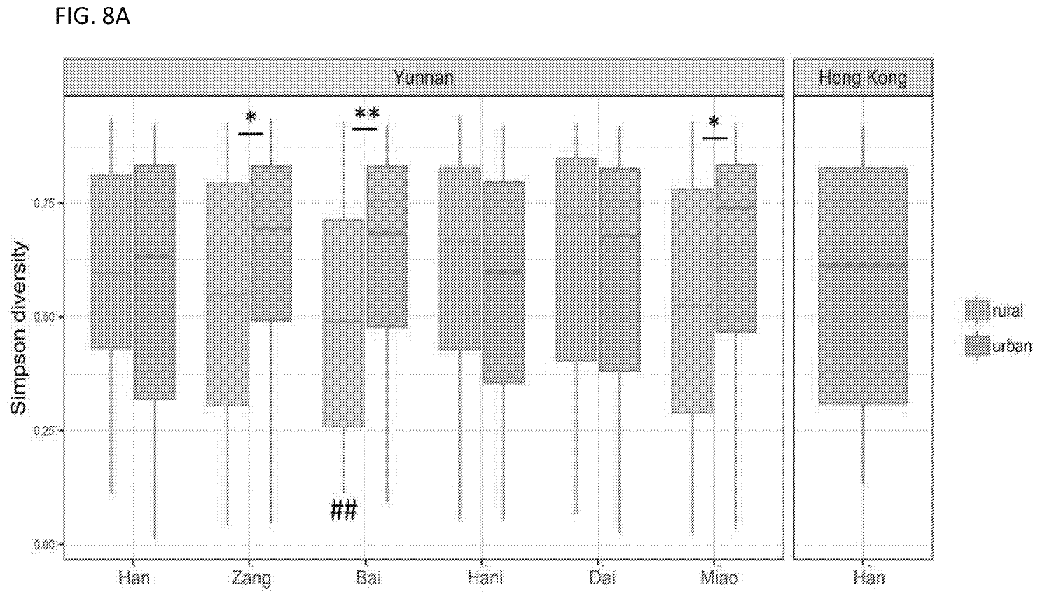

Dietary habit factors, including frequency of meat and vegetables, and wild food consumption (commonly seen in Yunnan), significantly impacted mycobiome composition variation (FIG. 6A). We conducted detailed diet recording and interrogated the effect of each dietary component on mycobiome variation. A total of 67 food choices (dietary components), characteristic of atypical Chinese foods comprising staple foods, side dishes (mostly cooked meats and vegetables), fruits and beverages, were included. Among the investigated populations, the Hong Kong versus the Yunnan population showed the most discriminatory dietary structure and differed significantly in consumption of side dishes and fruits (FIG. 7A). Butter milk tea and barley, purple rice and deep fried yak jerky, and sticky rice were characteristics of the habitual diet of the ethnicities Zang, Hani and Dai, respectively. 24 dietary components showed marked influence on mycobiome variations (FDR<5%, FIG. 7B). MaAsLin2 (Multivariate Association with Linear Models) analysis identified 28 food-fungal species correlations, among which blueberry and butter milk tea showed the largest number of food-fungal correlations.

### Diversity of Gut Mycobiome Across Populations

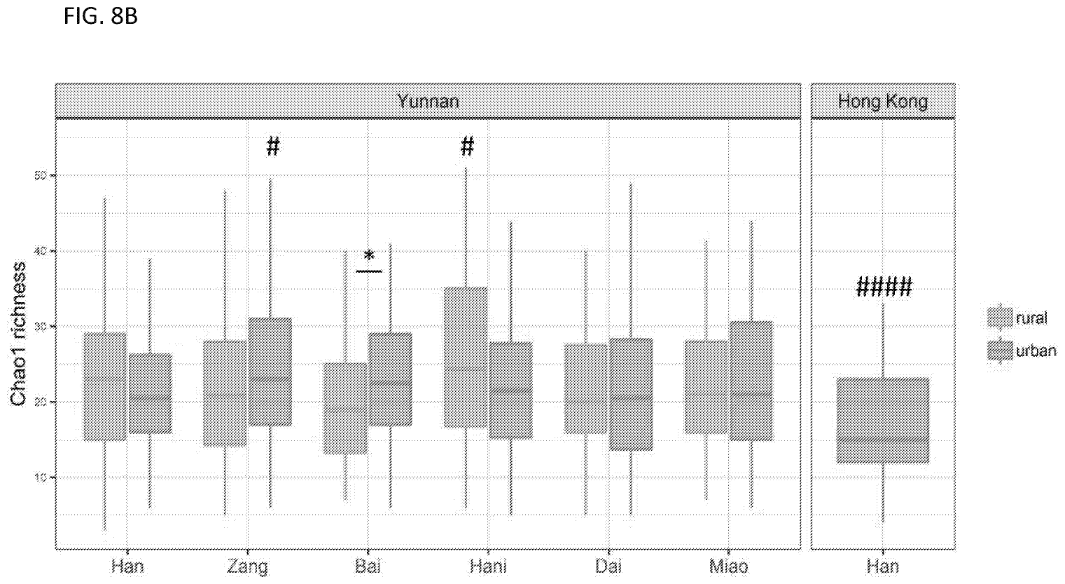

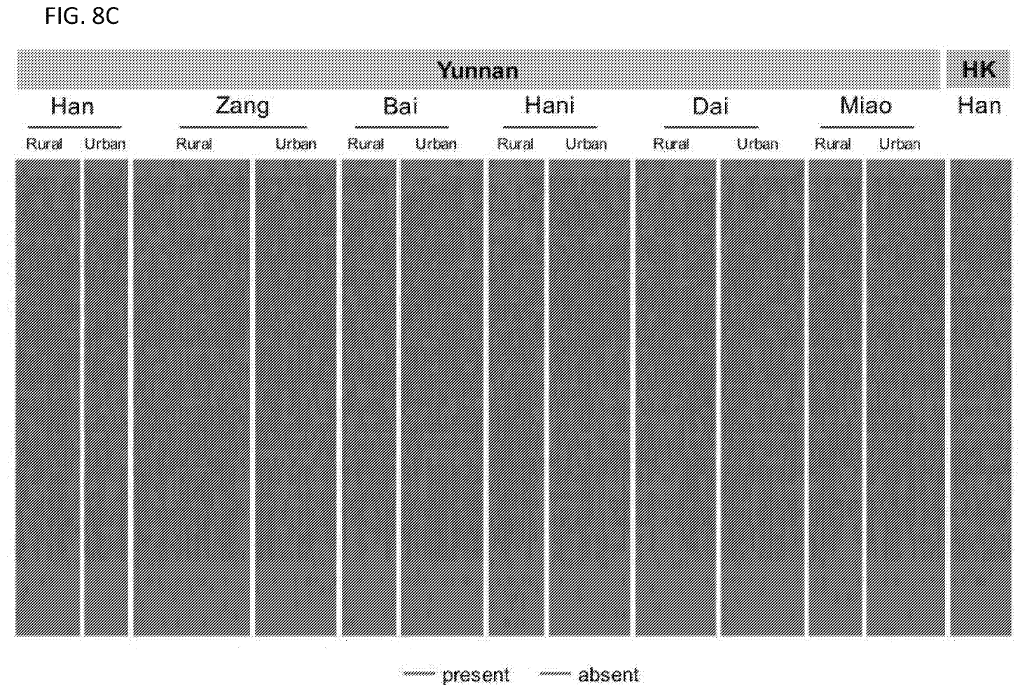

Urbanisation has been associated with a decrease in bacterial microbiome diversity, which were central to the increase of chronic diseases worldwide. We therefore examined the a diversity (diversity and richness) of the gut mycobiome and found that fungal community diversity (Simpson diversity index) was significantly increased in urban residents of ethnic groups Zang, Bai and Miao when compared with their rural counterparts (Mann-Whitney test, p values<0.05, <0.01 and <0.05, respectively; FIG. 8A). The rural Bai population showed the lowest gut mycobiome diversity amongst all studied Chinese populations (one-way anova, Holm-Bonferrroni adjusted p<0.01, FIG. 8A). In addition, there was a significant increase in the mycobiome species richness of urban Bai residents compared with their rural counterparts (Chao1 richness, Mann-Whitney test, p<0.01, FIG. 8B). Whilst rural Hani and urban Zang populations showed higher mycobiome richness compared to other Chinese populations (one way anova, Holm-Bonferrroni adjusted p<0.05, FIG. 8B), the Hong Kong population showed the lowest mycobiome richness (p<0.0001, FIG. 8B), with a marked depletion of multiple fungal species (FIG. 8C). Our findings underscored the importance of geography and environmental factors in shaping the α diversity of the gut mycobiome, and highlighted that urbanization was associated with an altered gut mycobiome diversity and the effect may be population- and ethnicity-dependent, in line with findings reported for the gut bacterial microbiome.

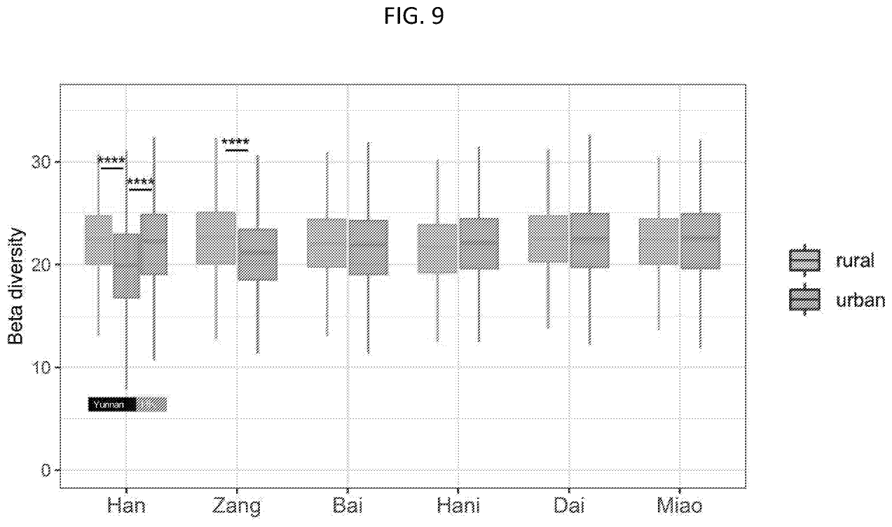

In Yunnan, the urban mycobiome of Han and Zang ethnic groups displayed significantly lower inter-individual mycobiome dissimilarity (beta-diversity) compared to their respective rural counterpart (t test, both p<0.0001, FIG. 9), suggesting that urbanisation homogenized the gut mycobiome composition amongst these groups given the multi-ethnicity residential feature in Kunming (urban city of Yunnan, China). However, the inter-individual mycobiome dissimilarity among the Han Hong Kong population (highly urbanized city, China) was higher than that of the urban Han population in Yunnan (t test, p<0.0001, FIG. 9), which may be related to higher standards of hygiene and sanitation thereby limiting inter-individual microbial transmission.

### Urbanization-, Geography- and Ethnicity-Specific Variations in Gut Mycobiome

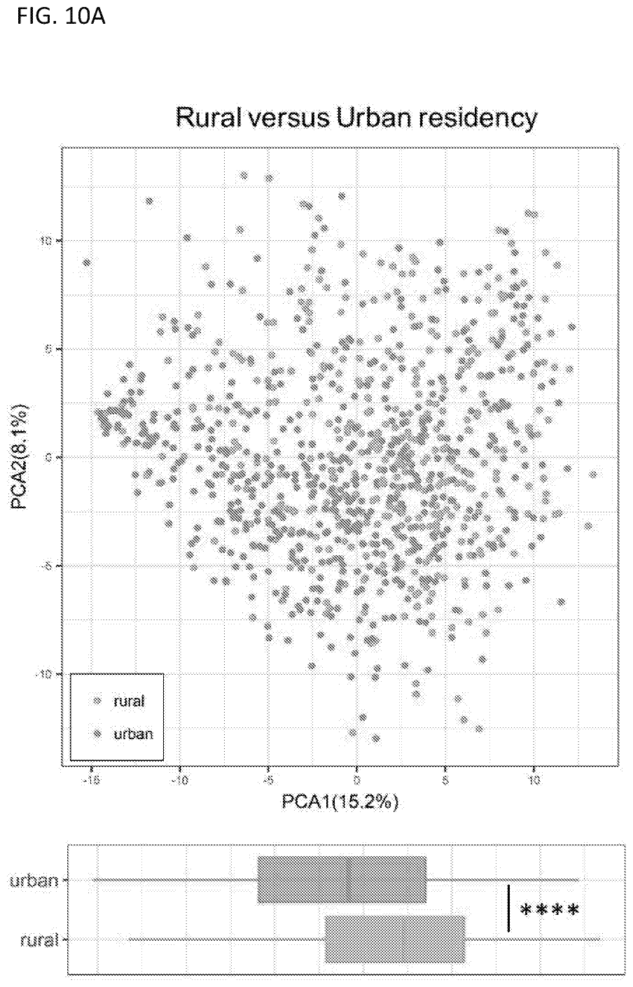

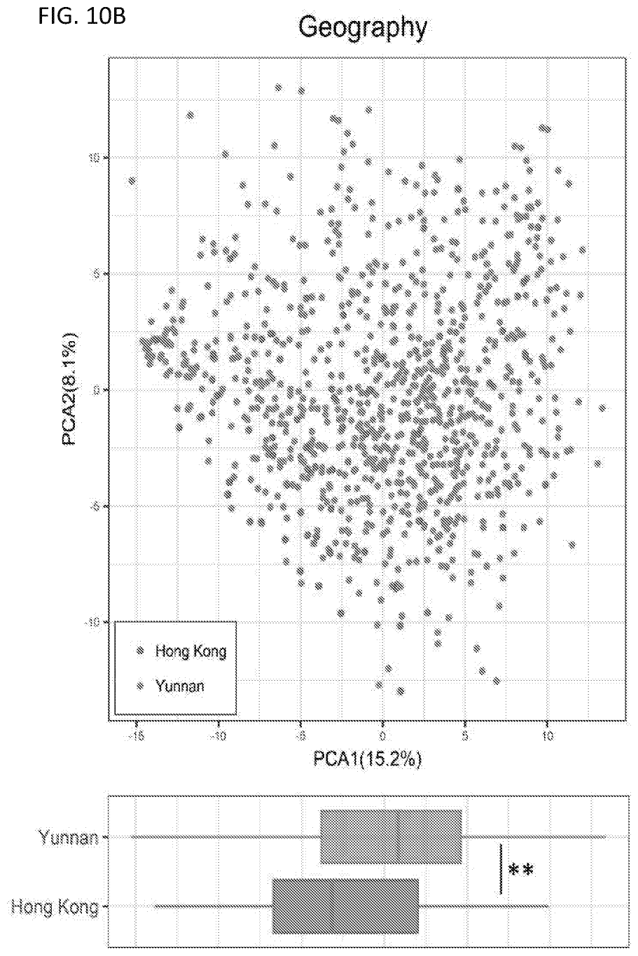

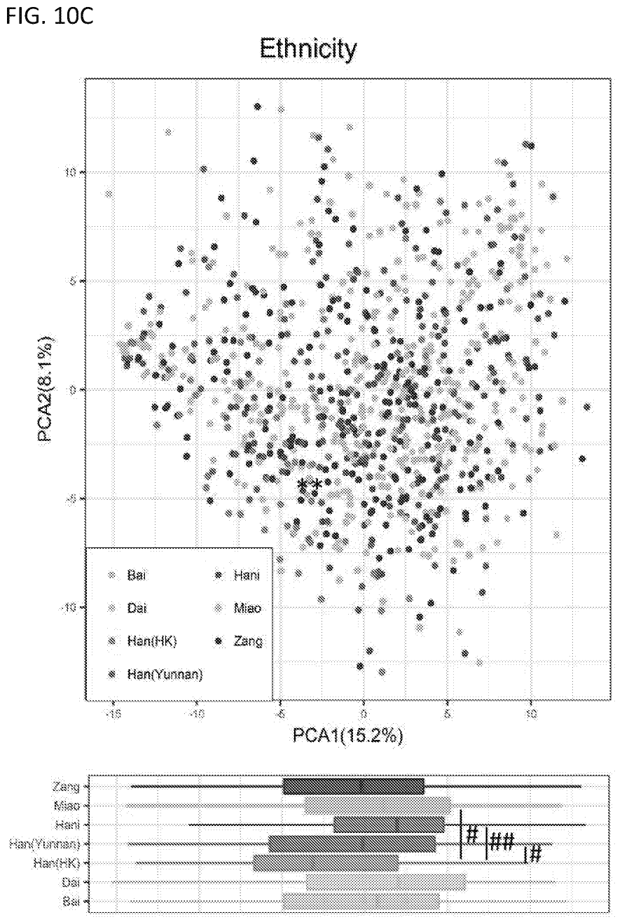

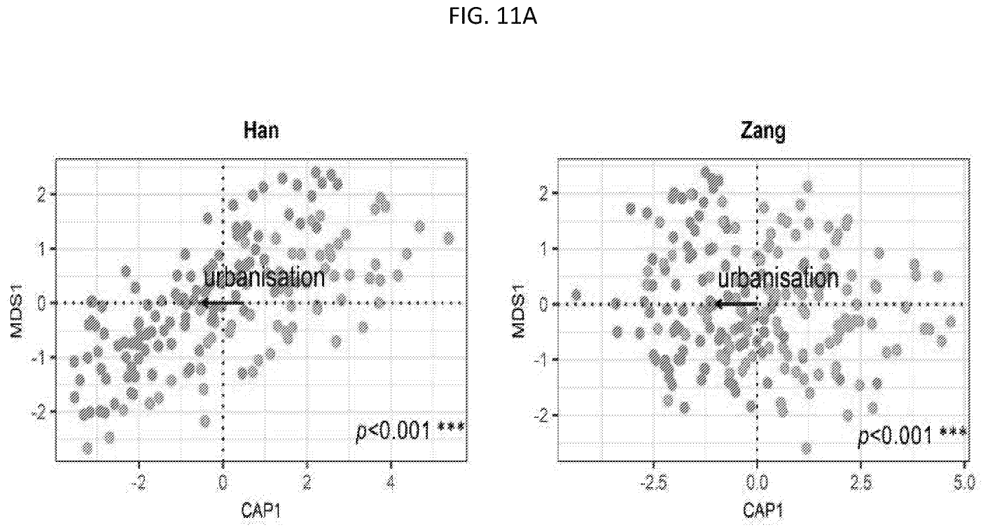

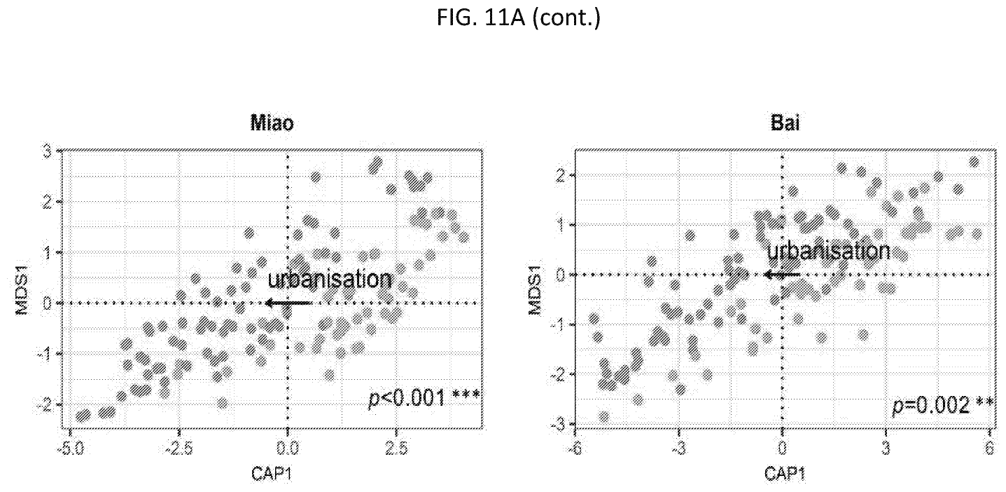

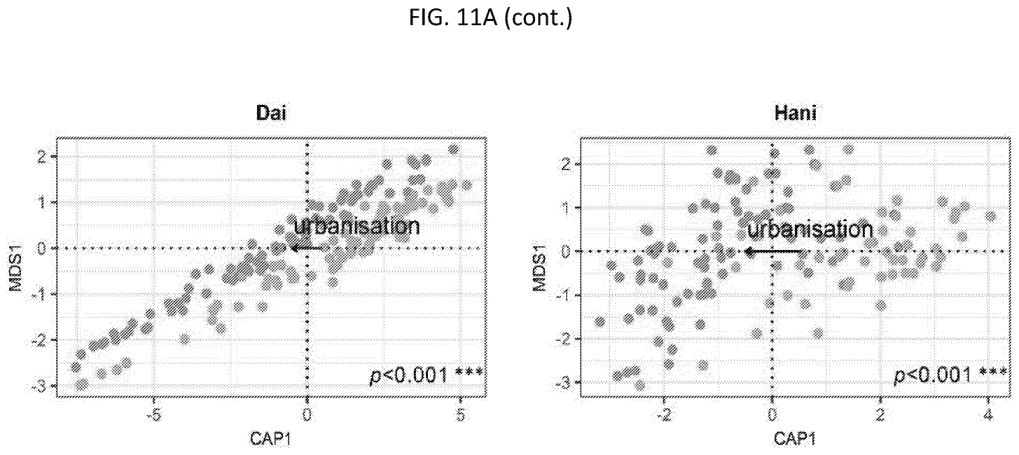

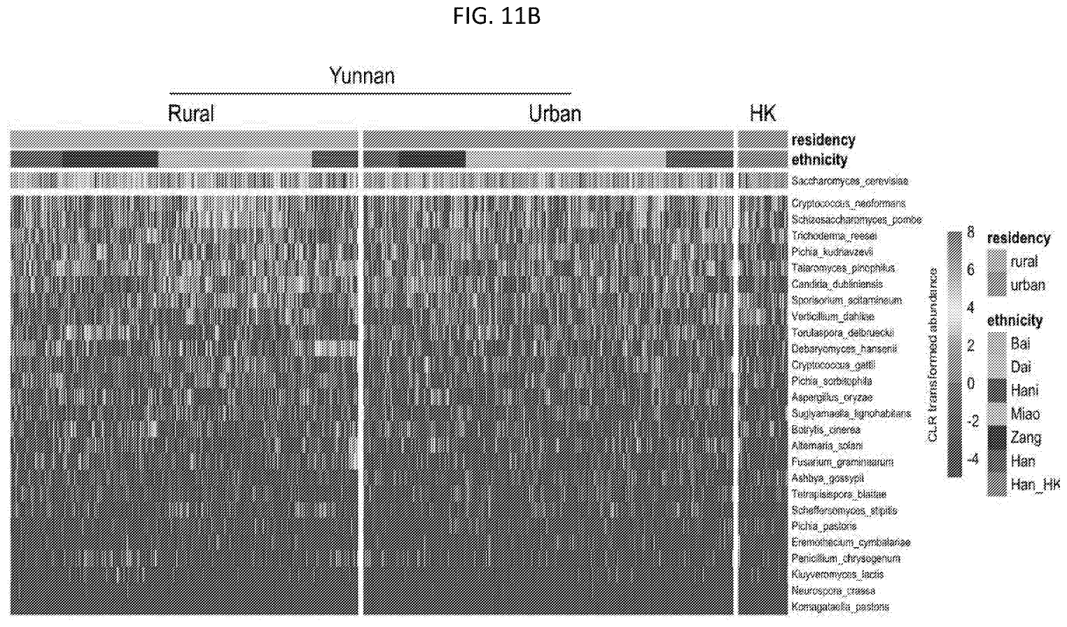

To determine the variation of gut mycobiome with respect to rural versus urban residency, geography, and ethnicity, we performed principal component analysis (PCA) on the fungal species-level community profiles. Rural versus urban residency significantly contributed to population gut mycobiome variations (t test on the dispersion of rural versus urban mycobiomes on the axis PC1, p<0.0001, FIG. 10A), which was further corroborated by the observation that urban residence significantly shifted the mycobiome configuration for all six ethnic groups in Yunnan (Distance-based redundancy analysis, Capscale test, all p<0.01, FIG. 11A). To identify gut fungal taxa associated with urbanisation, we performed LefSE (Linear discriminant analysis Effect Size) analysis on the fecal mycobiomes between the rural and urban populations in Yunnan. Saccharomyces cerevisiae was the only species enriched in urban subjects whereas 26 fungal species were enriched in rural subjects (FIG. 11B). The universally high presentation of Saccharomyces cerevisiae in the Hong Kong population and relatively higher representation of Saccharomyces cerevisiae in urban than rural Yunnan populations (FIG. 11B) suggests that Saccharomyces cerevisiae in the human gut may a characteristic of urban living.

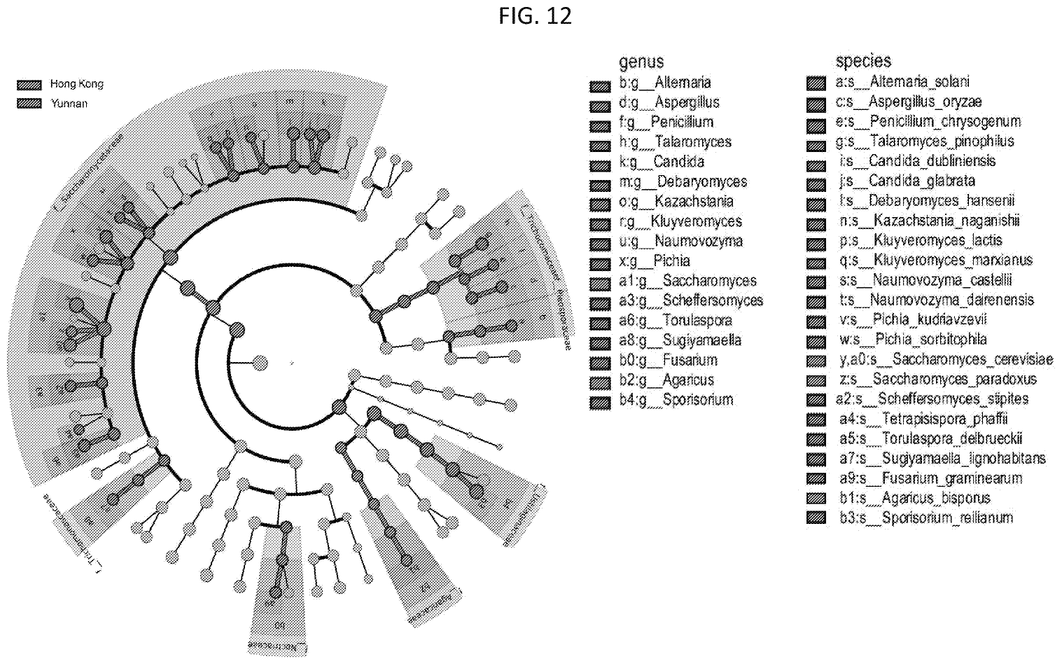

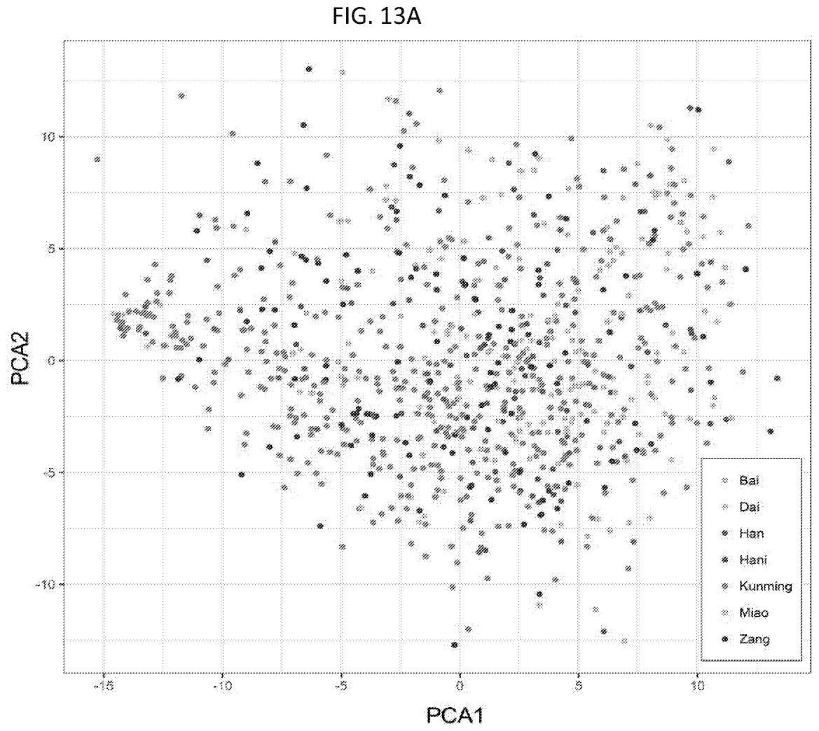

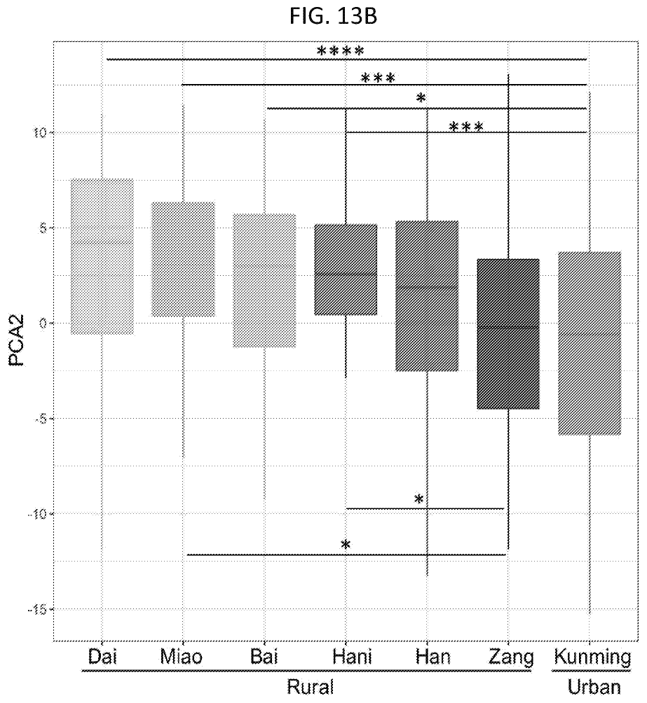

With regard to geography, the gut mycobiome of Hong Kong residents were highly variable and significantly separated from the mycobiomes of Yunnan residents (as reflected along the PC1 axis, t test, p<0.01, FIG. 10B). The gut mycobiome of the ethnic groups Miao, Hani and Han in Yunnan differed significantly from those of the gut mycobiome of Han in Hong Kong (one-way anova with Tukey's HSD test on PC1, all Holm-Bonferrroni adjusted p<0.05, FIG. 10C). To determine the compositional difference between Yunnan and Hong Kong populations, we conducted LefSE analysis and found that the Hong Kong population was enriched for the species Saccharomyces cerevisiae, Saccharomyces paradoxus, and Agaricus bisporus, whereas Yunnan populations were enriched for more diverse fungal species from multiple genera (all LefSE effect sizes >2 and FDR adjusted p<0.05, FIG. 12). We hypothesized that living in different areas within Yunnan (the urban city Kunming versus rural districts) could also affect the gut mycobiome composition. Residence in Kunming (urban) versus various rural districts accounted for 1.9% of the gut mycobiome variation among Yunnan populations (PERMANOVA test, p<0.001). Subjects from urban Yunnan showed the most distinct mycobiome configuration from other rural Yunnan groups (FIGS. 13A and 13B). These data further highlight the combined effects of urbanisation, geography, and ethnicity on influencing gut mycobiome composition.

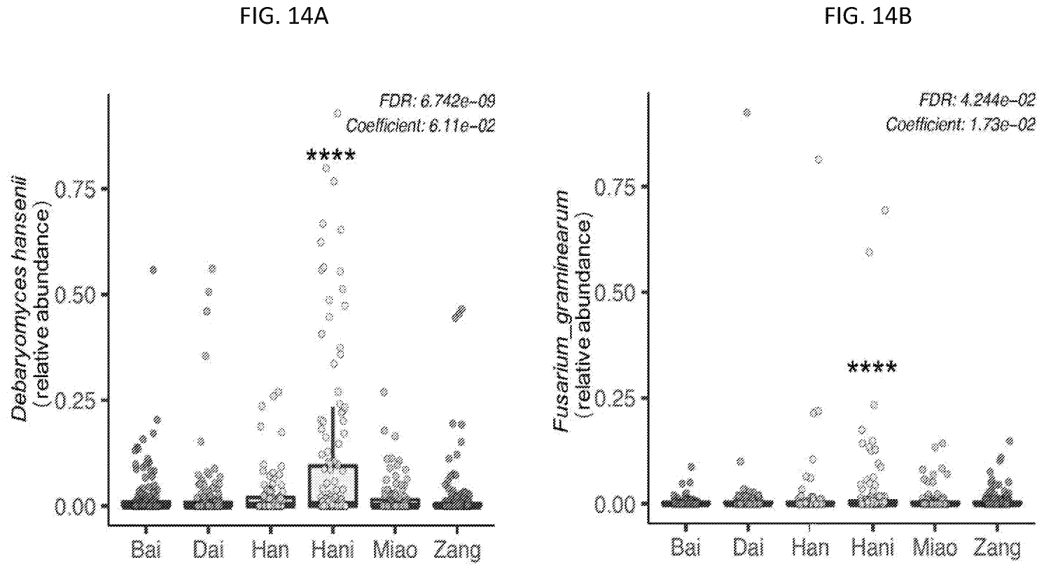

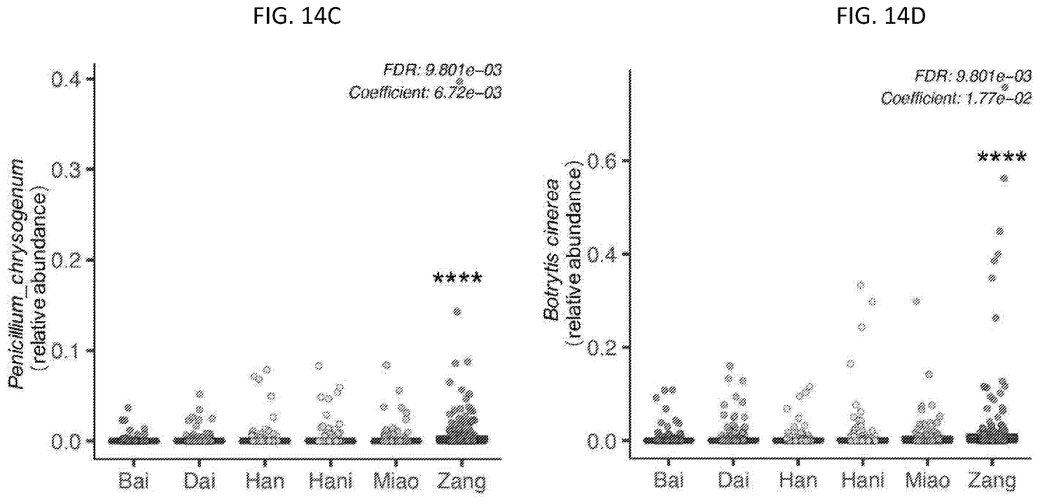

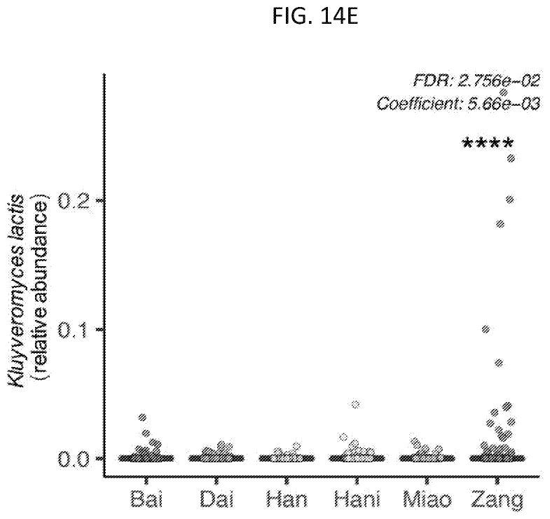

To determine ethnicity-specific fungal features, we performed MaAsLin2 analysis on the mycobiome compositions of the six ethnic groups from Yunnan (Table 5). The gut mycobiome of Zang and Hani differed remarkably from that of other ethnic groups. Botrytis cinerea (a plant pathogen), Penicillium chrysogenum and Kluyveromyces lactis (lactose converter) were overrepresented in Zang, whereas Debaryomyces hansenii and Fusarium graminearum (both plant pathogens) were enriched in Hani (all FDR adjusted p<0.05, FIGS. 14A-14E). Among these species, Botrytis cinerea and Penicillium chrysogenum correlated with consumption of Zang-specific dietary component, butter milk tea (Table 5 and FIG. 7A). Kluyveromyces lactis is a yeast capable of assimilating and metabolizing lactose, which is a component highly enriched in butter milk tea. Fusarium graminearum is a plant associated fungus in small grains, including rice and wheat. The habitual diet of Hani consisting of purple rice, sticky rice and wheat (FIG. 7A) coincided with the enrichment of Fusarium graminearum in Hani's gut mycobiomes. Altogether, our data highlight for the first time dietary components may impact the configuration and divergence of a population's gut mycobiome.

TABLE 5

| Dietary | | | | | | | |
|---|---|---|---|---|---|---|---|
| component | fungus | coef | stderr | N | N.not.0 | pval | qval |
| Chinese | Fusarium_graminearum | 0.097975569 | 0.011891 | 738 | 298 | 9.11E−16 | 1.37E−12 |
| cabbage | | | | | | | |
| brown rice | Lachancea_thermotolerans | 0.000850244 | 0.000112 | 738 | 179 | 1.25E−13 | 1.25E−10 |
| blueberry | Tetrapisispora_blattae | 0.018987995 | 0.002735 | 738 | 272 | 9.15E−12 | 6.89E−09 |
| Deep fried | Debaryomyces_hansenii | 0.020665279 | 0.003324 | 738 | 396 | 8.96E−10 | 5.40E−07 |
| Yak Jerky | | | | | | | |
| blueberry | Sugiyamaella_lignohabitans | 0.025006359 | 0.004324 | 738 | 370 | 1.12E−08 | 5.65E−06 |
| Cherry | Eremothecium_sinecaudum | 0.001965643 | 0.000343 | 738 | 214 | 1.46E−08 | 6.28E−06 |
| Butter Milk | Naumovozyma_castellii | 0.005014038 | 0.000886 | 738 | 285 | 2.24E−08 | 8.44E−06 |
| Tea | | | | | | | |
| cantaloupe | Verticillium_dahliae | 0.006386132 | 0.001273 | 738 | 441 | 6.76E−07 | 0.000227 |
| chicken | Debaryomyces_hansenii | 0.02384679 | 0.005074 | 738 | 396 | 3.16E−06 | 0.00088 |
| coffee | Saccharomyces_paradoxus | 0.001338922 | 0.000285 | 738 | 254 | 3.21E−06 | 0.00088 |
| brown rice | Tetrapisispora_blattae | 0.002012133 | 0.000434 | 738 | 272 | 4.26E−06 | 0.001071 |
| duck | Multicellular_Multicellular | 0.003574602 | 0.000785 | 738 | 267 | 6.21E−06 | 0.00144 |
| Fig | Sporisorium_scitamineum | 0.006535619 | 0.001443 | 738 | 499 | 7.01E−06 | 0.00151 |
| Longan | Verticillium_dahliae | 0.005519635 | 0.001302 | 738 | 441 | 2.54E−05 | 0.005105 |
| Butter Milk | Botrytis_cinerea | 0.009227271 | 0.002226 | 738 | 312 | 3.85E−05 | 0.007246 |
| Tea | | | | | | | |
| watermelon | Scheffersomyces_stipitis | 0.005718153 | 0.001396 | 738 | 274 | 4.69E−05 | 0.008316 |
| blueberry | Kazachstania_africana | 0.009857631 | 0.002452 | 738 | 241 | 6.46E−05 | 0.010257 |
| grapefruit | Zygosaccharomyces_parabailii | 0.009479301 | 0.002358 | 738 | 343 | 6.46E−05 | 0.010257 |
| Butter Milk | Penicillium_chrysogenum | 0.003380742 | 0.000861 | 738 | 166 | 9.51E−05 | 0.014334 |
| Tea | | | | | | | |
| Chinese | Candida_glabrata | 0.029962018 | 0.007686 | 738 | 213 | 0.000107 | 0.014606 |
| date | | | | | | | |
| Plum | Multicellular_Multicellular | 0.002916072 | 0.000748 | 738 | 267 | 0.000106 | 0.014606 |
| jackfruit | Eremothecium_cymbalariae | 0.000725819 | 0.000188 | 738 | 227 | 0.000124 | 0.016297 |
| grapefruit | Candida_glabrata | 0.011598114 | 0.003153 | 738 | 213 | 0.000254 | 0.031854 |
| blueberry | Kazachstania_naganishii | 0.003869982 | 0.001061 | 738 | 246 | 0.000284 | 0.0343 |
| Plum | Sclerotinia_sclerotiorum | 0.002703897 | 0.000747 | 738 | 296 | 0.000316 | 0.036668 |
| Durian | Lachancea_thermotolerans | 0.000434429 | 0.000121 | 738 | 179 | 0.000362 | 0.040447 |
| Ham | Naumovozyma_dairenensis | 0.001692468 | 0.000474 | 738 | 239 | 0.000382 | 0.041091 |
| papaya | Saccharomycopsis_fibuligera | 0.001520144 | 0.000431 | 738 | 345 | 0.000444 | 0.046201 |

### Correlation Between Gut Mycobiome and Blood Parameters

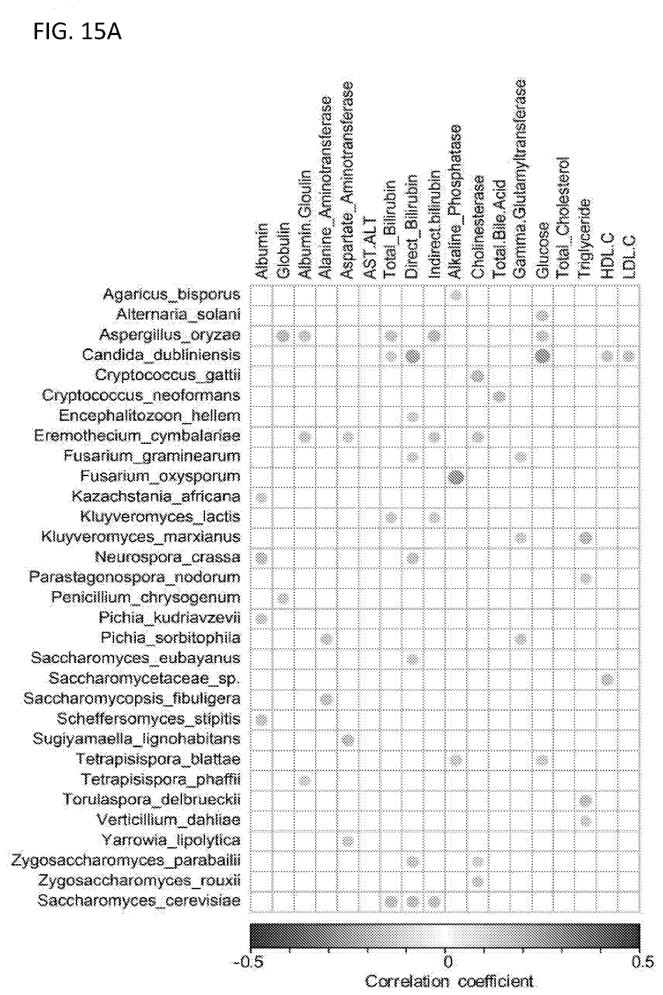

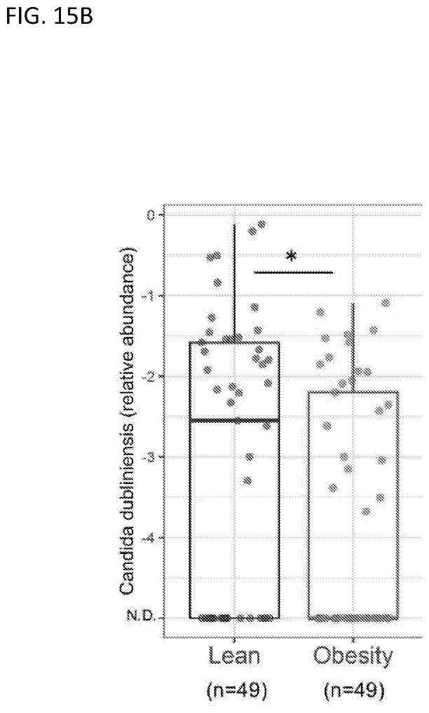

We clinically profiled the blood biochemical parameters and correlated them with the gut mycobiome profile (FIGS. 15A and 15B). Among all significant fungus-blood parameter correlations, Candida dubliniensis exhibited the strongest inverse correlation with blood glucose. Furthermore, we also found that Candida dubliniensis showed a positive correlation with high-density lipoprotein cholesterol (HDL-C) and an inverse correlation with low-density lipoprotein cholesterol (LDL-C) (FIGS. 15A and 15B). This data suggests that Candida dubliniensis have a putative role associated with protection from metabolic diseases.

### Trans-Kingdom Interactions Between Gut Fungi and Bacteria

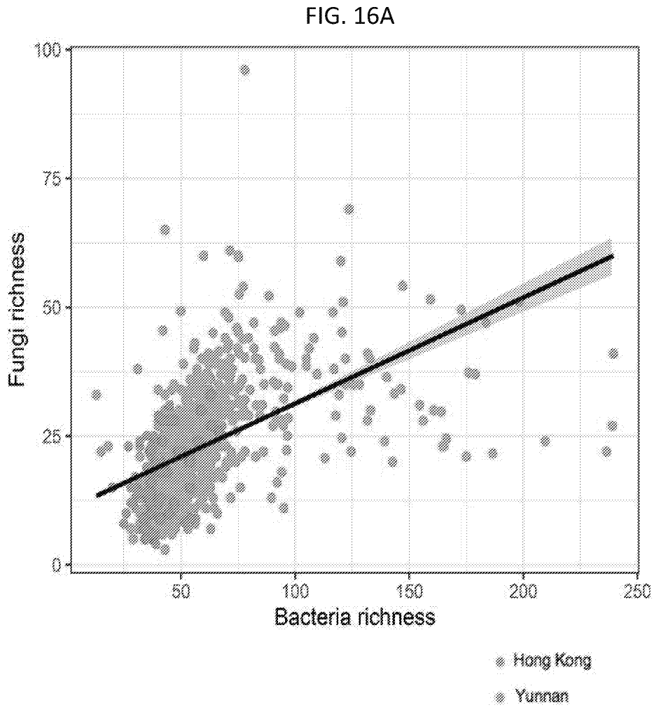

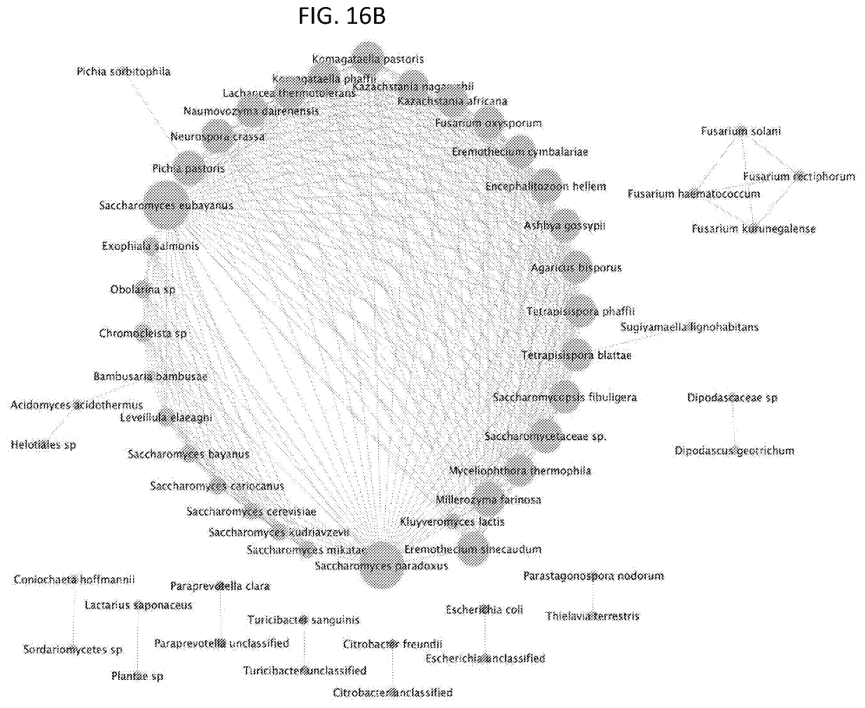

We next explored the trans-kingdom associations between the gut fungi and bacteria. A significant positive correlation was identified between fungi richness and bacteria richness (Pearson correlation Rho=0.509, p<−2.2e-16, FIG. 16A), indicating a mutualistic relationship between the fungal and bacterial species within the human gut ecosystem. At the species level, we analyzed trans-kingdom interactions across fungal and bacterial taxa based on their relative abundance profile. Through SpeciEasi correlation analysis, we observed strong within-kingdom positive correlations but rare transkingdom correlations between fungi and bacteria (FIG. 16B). The fungal species Saccharomyces eubayanus and Saccharomyces paradoxus were the most inter-connected fungal species. Interestingly, most inter-connected fungi were members from the same genus or family, such as the genera Saccharomyces and Fusarium (FIG. 16B). These data suggests that fungal members from the same genus or family may have similar niche- and nutrient-preference in the human gut.

We showed for the first time the impact of geography, ethnicity and urbanization on human gut mycobiome composition using population-based ultradeep shotgun metagenome sequencing. Host metadata and dietary factors exhibited substantial effects in mycobiome variations. Similar to other metadata-bacteriome association studies, the gut mycobiome covariates had a cumulative, non-redundant effect size of 9.8%. These data suggest the influence of additional, currently unknown covariates as well as intrinsic microbial ecological factors. Only a small proportion of the population had medication exposures in the recent 3 months resulting in no significant effects observed for each medicinal variable; however medication overall exerted an effect size of 1.5% on population mycobiome variations. Factors related to urbanization showed the strongest effect, followed by geography, dietary habit and ethnicity. Our study suggests that future investigations on human mycobiome should consider rural/urban, geographical, and ethnic effects. Consistent with the prevailing hypothesis that urbanisation is associated with depletion of gut bacteria, we found that individuals living in the highly urbanized region, Hong Kong, had a decreased richness of the gut mycobiome. Changes in environment and residency region are inevitable with increasing urbanisation and population immigration, which are often associated with risks for certain diseases, such as obesity, childhood allergies, diabetes mellitus, and inflammatory bowel disease. Given the pathogenesis of such diseases are related to alterations in the gut microbiome, further studies regarding the functional consequences of disparate mycobiome configurations merit in-depth investigation.

### REFERENCES

1. Deschasaux, M., et al. Depicting the composition of gut microbiota in a population with varied ethnic origins but shared geography. Nat Med 24, 1526 (2018).
2. Falony, G., et al. Population-level analysis of gut microbiome variation. Science 352, 560-564 (2016).
3. He, Y., et al. Regional variation limits applications of healthy gut microbiome reference ranges and disease models. Nat Med 24, 1532 (2018).
4. Lynch, S. V. & Pedersen, O. The human intestinal microbiome in health and disease. New Engl J Med 375, 2369-2379 (2016).
5. Yatsunenko, T., et al. Human gut microbiome viewed across age and geography. Nature 486, 222 (2012).
6. Zuo, T., Kamm, M. A., Colombel, J.-F. & Ng, S. C. Urbanization and the gut microbiota in health and inflammatory bowel disease. Nat Rev Gastro Hepat 15, 440
7. Blaser, M. J. The theory of disappearing microbiota and the epidemics of chronic diseases. Nat Rev Immunol 17, 461 (2017).
8. Vangay, P., et al. US immigration westernizes the human gut microbiome. Cell 175, 962-972. e910 (2018).
9. Faust, K. & Raes, J. Microbial interactions: from networks to models. Nat Rev Microbiol 10, 538-550 (2012).
10. Salminen, S., Gibson, G. R., McCartney, A. L. & Isolauri, E. Influence of mode of delivery on gut microbiota composition in seven year old children. Gut 53, 1388-1389 (2004).
11. Baumann-Dudenhoeffer, A. M., D'Souza, A. W., Tarr, P. I., Warner, B. B. & Dantas, G. Infant diet and maternal gestational weight gain predict early metabolic maturation of gut microbiomes. Nat Med 24, 1822-1829 (2018).
12. Sonnenburg, J. L. & Sonnenburg, E. D. Vulnerability of the industrialized microbiota. Science 366, eaaw9255 (2019).
13. Gaulke, C. A. & Sharpton, T. J. The influence of ethnicity and geography on human gut microbiome composition. Nat Med 24, 1495 (2018).
14. Rothschild, D., et al. Environment dominates over host genetics in shaping human gut microbiota. Nature 555, 210 (2018).
15. Martínez-Villaluenga, C., Cardelle-Cobas, A., Corzo, N., Olano, A. & Villamiel, M. Optimization of conditions for galactooligosaccharide synthesis during lactose hydrolysis by β-galactosidase from Kluyveromyces lactis (Lactozym 3000 L HP G). Food Chem 107, 258-264 (2008).
16. Dickson, R. C. & Barr, K. Characterization of lactose transport in Kluyveromyces lactis. J Bacteriol 154, 1245-1251 (1983).
17. Lee, J., et al. Genetic diversity and fitness of Fusarium graminearum populations from rice in Korea. Appl. Environ. Microbiol. 75, 3289-3295 (2009).
18. Kaplan, G. G. & Ng, S. C. Globalisation of inflammatory bowel disease: perspectives from the evolution of inflammatory bowel disease in the UK and China. The Lancet Gastroenterology & Hepatology 1, 307-316 (2016).
19. Cheema, A., Adeloye, D., Sidhu, S., Sridhar, D. & Chan, K. Y. Urbanization and prevalence of type 2 diabetes in Southern Asia: A systematic analysis. J Glob Health 4(2014).
20. Swinburn, B. A., et al. The global obesity pandemic: shaped by global drivers and local environments. The Lancet 378, 804-814 (2011).
21. Zuo, T. & Ng, S. C. The gut microbiota in the pathogenesis and therapeutics of inflammatory bowel disease. Frontiers in microbiology 9(2018).
22. Hartstra, A. V., Bouter, K. E. C., Bäckhed, F. & Nieuwdorp, M. Insights into the role of the microbiome in obesity and type 2 diabetes. Diabetes care 38, 159-165 (2015).
23. Ni, J., Wu, G. D., Albenberg, L. & Tomov, V. T. Gut microbiota and IBD: causation or correlation? Nat Rev Gastro Hepat 14, 573 (2017).
24. Turnbaugh, P. J., et al. An obesity-associated gut microbiome with increased capacity for energy harvest. Nature 444, 1027 (2006).
25. Bolger, A. M., Lohse, M. & Usadel, B. Trimmomatic: a flexible trimmer for Illumina sequence data. Bioinformatics 30, 2114-2120 (2014).
26. Segata, N., et al. Metagenomic biomarker discovery and explanation. Genome biology 12, R60 (2011).
27. Langmead, B. & Salzberg, S. L. Fast gapped-read alignment with Bowtie 2. Nature methods 9, 357 (2012).
28. Soverini, M., et al. HumanMycobiomeScan: a new bioinformatics tool for the characterization of the fungal fraction in metagenomic samples. BMC genomics 20, 496 (2019).
29. Oksanen, J., et al. Vegan: community ecology package. R package version 1.17-4. 2010
30. Benjamini, Y. & Hochberg, Y. Controlling the false discovery rate: a practical and powerful approach to multiple testing. Journal of the Royal statistical society: series B (Methodological) 57, 289-300 (1995).
31. Clarke, K. R. & Ainsworth, M. A method of linking multivariate community structure to environmental variables. Marine Ecology-Progress Series 92, 205-205 (1993).
32. Gloor, G. B., Macklaim, J. M., Pawlowsky-Glahn, V. & Egozcue, J. J. Microbiome datasets are compositional: and this is not optional. Frontiers in microbiology 8, 2224 (2017).
33. Aitchison, J. The statistical analysis of compositional data. Journal of the Royal Statistical Society: Series B (Methodological) 44, 139-160 (1982).
34. Kurtz, Z. D., et al. Sparse and compositionally robust inference of microbial ecological networks. PLoS computational biology 11(2015).
35. Lovell, D., Pawlowsky-Glahn, V., Egozcue, J. J., Marguerat, S. & Bähler, J. Proportionality: a valid alternative to correlation for relative data. PLoS computational biology 11(2015).
36. Erb, I. & Notredame, C. How should we measure proportionality on relative gene expression data? Theory in Biosciences 135, 21-36 (2016).
37. Shannon, P., et al. Cytoscape: a software environment for integrated models of biomolecular interaction networks. Genome Res 13, 2498-2504 (2003).
38. S C Ng, H Y Shi, N Hamidi, F E Underwood, W Tang, E I Benchimol, R Panaccione, S Ghosh, J C Y Wu, F K L Chan, J J Y Sung, and GG Kaplan, Worldwide incidence and prevalence of inflammatory bowel disease in the 21st century: a systematic review of population-based studies. Lancet, 12, 2017. 390 (10114): p. 2769-2778.
39. T Zuo, X J Lu, Y Zhang, C P Cheung, S Lam, F Zhang, W Tang, J Y L Ching, R Zhao, P K S Chan, J J Y Sung, J Yu, F K L Chan, Q Cao, J Q Sheng, and S C Ng. Gut mucosal virome alterations in ulcerative colitis. Gut, 07, 2019. 68 (7): p. 1169-1179.
40. T Zuo, S H Wong, CP Cheung, K Lam, R Lui, K Cheung, F Zhang, W Tang, J Y L Ching, J C Y Wu, P K S Chan, J J Y Sung, J Yu, F K L Chan, and S C Ng. Gut fungal dysbiosis correlates with reduced efficacy of fecal microbiota transplantation in Clostridium difficile infection. Nature Communications, 09, 2018. 9 (1): p. 3663.
41. C Y Lai, J Sung, F Cheng, W Tang, S H Wong, P K S Chan, M A Kamm, J J Y Sung, G Kaplan, F K L Chan, and S C Ng, Systematic review with meta-analysis: review of donor features, procedures and outcomes in 168 clinical studies of faecal microbiota transplantation. Alimentary Pharmacology and Therapeutics, 02, 2019. 49 (4): p. 354-363
42. T Zuo, S H Wong, K Lam, R Lui, K Cheung, W Tang, J Y L Ching, P K S Chan, M C W Chan, J C Y Wu, F K L Chan, J Yu, J J Y Sung, and S C Ng, Bacteriophage transfer during faecal microbiota transplantation in Clostridium difficile infection is associated with treatment outcome. Gut, 04, 2017. 67 (4): p. 634-643.
43. Norman, J. M. et al. Disease-Specific Alterations in the Enteric Virome in Inflammatory Bowel Disease. Cell 160, 447-460 (2015).
44. Zuo, T. et al. Bacteriophage transfer during faecal microbiota transplantation in Clostridium difficile infection is associated with treatment outcome. Gut 313952 (2017).

### Example III

### Background

Emerging data have highlighted the potential role of the gut microbiome in influencing systemic metabolism and the development of diabetes and obesity. Since not every obese subject has underlying T2DM, whether having both conditions are associated with more perturbed gut microbiota is not clear. It was reported that obese subjects without T2DM (Ob) had more severe bacterial microbiome variation than obese subjects with T2DM (ObT2) compared to the lean controls. These results indicate that gut microbes play a distinct role in both T2DM and obesity.

The gut viral community (virome), a critical component of the human gut microbiome, is highly diverse but understudied. It is dominated by prokaryotic viruses also called bacteriophages (phages), which are viruses that attack bacteria in a host-specific manner. Recent evidence has mounted that the gut virome plays a key role in shaping the composition of the gut microbiota, and several studies have demonstrated a role of the gut virome autoimmune and inflammatory gut diseases. Increased abundance of gut phages has also been linked to T2DM. A proof-of-concept study demonstrated that fecal virome transplantation (FVT) from lean donors was effective in shifting the phenotype of obese mice to resemble lean mice. In this study, the present inventors have hypothesized that gut virome composition differs between obese and lean subjects, and the presence of T2DM is associated with further alterations of gut virome composition. The inventors performed deep shotgun metagenomic sequencing of virus like particles (VLP)-derived DNA and total bulk DNA in fecal samples to characterize the gut virome and bacteriome, respectively, in subjects with obesity and T2DM.

### Methods

### Study Cohort and Sample Collection

229 adult subjects (obese 128; lean controls 101) were recruited from two regions in China (HK: Hong Kong and KM: Kunming) and collected clinical metadata including age, gender, body mass index (BMI), T2DM, alcohol intake, smoking and medications. Lean healthy controls were recruited from the general population through advertisement and were included if they had a BMI≥18.5 and <23 kg/m². Obese subjects were recruited from bariatric clinics and were included if they had a BMI≥28 kg/m², and had no severe gastrointestinal diseases (inflammatory bowel diseases, cancer, advanced adenoma), autoimmune diseases, active infection, acquired immunodeficiency syndrome, known history of organ dysfunction or failure, abdominal surgery, radio-chemotherapy, immunotherapy or current incurable cancer. Fecal samples were collected and stored at −80° C. for gut virome and bacteriome analysis. Ethical approval and written informed consents were obtained from all study subjects.

### Fecal Virus Like Particles (VLP) DNA Extraction and Sequencing

Based on methods described from previous published studies, VLPs were extracted by following steps^{17,22}. 100-200 mg of stool was added into 400 μl saline-magnesium buffer, then vortexing for 10 min. Suspensions were then centrifugated at 2,000×g to remove the debris and cells. The supernatant from previous suspensions was passed through 0.45 μm and 0.22 μm filters, to remove large particles including residual host cells and bacteria. To remove residual bacterial and host cell membranes, samples were treated with lysozyme (1 mg/ml at 37° C. for 30 min) followed by chloroform (0.2× volume at RT for 10 min). Non-virus protected DNA was removed by treatment with 1U Baseline zero DNase (Epicenter) followed by heat inactivation of DNases at 65° C. for 10 min. To extract nucleic acid from VLP, samples were cleaved with 4% SDS plus 38 mg/ml proteinase K at 56° C. then treated with CTAB buffer and Phenol: Chloroform: Isoamyl Alcohol (pH 8.0). Aqueous portion was washed once with equal volume of chloroform, followed by concentration kit (DNA Clean & Concentrator TM 89-5, Zymo Research). VLP DNA was amplified for 2 hours using Phi29 polymerase before sequencing. DNA libraries were constructed through processes of end repairing, purification, and PCR amplification, and sequenced by Illumina Novaseq 6000 with paired-end 150 bp sequencing strategy by Novogene, Beijing, China.

### Fecal Whole DNA Extraction and Sequencing

Stool DNA was extracted using Maxwell® RSC PureFood GMO and Authentication Kit. To be brief, add 1 ml of CTAB buffer to stool samples (100 mg), and vortex for 30 s, then heat the solution at 95° C. for 5 min. Afterwards, the sample was thoroughly vortexed with beads (equal volume of 0.1 mm and 0.5 mm) at max speed for 15 minutes. Subsequently, 40 μl of proteinase K and 20 μl of RNase A were added, and then incubate the mixture at 70° C. for 10 minutes. Finally, the supernatant was centrifuged at 13,000×g for 5 minutes and then placed in a Maxwell® RSC instrument for DNA extraction. DNA libraries were constructed through the processes of end repairing, purification, and PCR amplification. After DNA libraries construction, DNA libraries were sequenced by Illumina Novaseq 6000 with paired-end 150 bp sequencing strategy by Novogene, Beijing, China.

### Sequence Reads Quality Control

Shotgun metagenomic reads were quality-filtered and decontaminated of human sequences using KneadData (v0.7.2). Java8 (v1.8.0_152-release), Bowtie2 (v2.3.4.3) and Trimmomatic (v0.39.1) were pre-installed to run KneadData. Any leading or trailing N-bases and other bases that had Phred quality scores of 3 or below were trimmed, sequence reads with less than average quality score of 15 using a 4-base sliding window were cut, and short sequence reads with 50 or fewer bases were removed. Adapter sequences in paired-end reads were then cut by checking for maximum mismatch count, simple and palindromic matches of 2, 10 and 30 bases, respectively, with a library of universal Illumina TruSeq3-PE-2.fa adapter sequences. Post quality-trimmed metagenomic reads were passed to Bowtie2 for host decontamination. End-to-end Bowtie-2 alignment with “very-sensitive” preset options was perform against an indexed human genome (hg38). Reads not aligned to the human genome were kept as clean reads.

### Viral Taxonomy Annotation

Paired end VLPs reads were assembled into contigs by Megahit (v1.0.3) and contigs with length larger than 1,000 bp were kept and the contigs at a 95% identity level were clustered using CD-HIT (v4.7) to generate a unique contigs reference database. Open Reading Frame (ORF) was extracted from the 95% identify level contigs by Glimmer3 (v 3.02), only ORFs passed threshold of 100 amino acids were kept. A standalone entire UniProt TrEMBL database for virus and phage reference proteins was download on Feb. 11, 2019. the ORFs which extracted from contigs were blastx to the UniProt TrEMBL database with e<10⁻⁵ by Diamond (v 0.9.24). To assign taxonomy for each contigs, a voting system was use to choose the best assignment at order, family, genus and species, resepectively^{22,23}. The taxonomy was kept only for contigs greater than one ORF per 10 kb to reduce false taxonomy assignment on the contigs with limited similarity. In parallel, the contigs were blasted to NCBI RefSeq genome reads downloaded at Nov. 5, 2019, and any contig assign to cellular organisms were removed. The whole DNA sequencing reads (after removed bacterial, fungal and archaea reads) were then aligned to the unique contig reference database by Bowtie2 to get reads count table for each sample. The mapped read counts, contig lengths and total read counts were used to normalize the original read counts to Reads Per Kilobase Million (RPKM) and exported for downstream analysis.

### Bacterial Taxonomy Annotation

For whole DNA metagenomes, Kraken2 (v2.0.8-beta) was used to generate a species-level community composition. The reference bacterial genome was downloaded from NCBI RefSeq on Nov. 5, 2019, and the database was built with default parameters. Each query was thereafter classified to a taxon with the highest total hits of k-mer matched by pruning the general taxonomic trees affiliated with mapped genomes.

### Statistical Analysis

Alpha, beta diversity was calculated with R package phyloseq and vegan. Data process and visualization were performed by R packages (dplyr, readr, stringr, ggplot2, aPCoA, pheatmap and ggsignif). Two-tailed Wilcoxon Rank Sum test and Kruskal-Wallis test was used to determine statistically significant difference for alpha diversity indices between groups. Multivariate association with linear models (MaAsLin2) was used to identify associations between clinical metadata and microbial abundance while controlling for confounders. Machine learning by random forest were performed to develop prediction models for classify diseases from controls by gut vriome profile and metadata. Receiver operating characteristic (ROC) analysis was performed with the area under the curve (AUC) to assess the performance of the prediction models. Inter-kingdom correlations were calculated by SparCC, and p value was corrected with false discovery rate (FDR). All statistic tests were done by R (v3.6.1) and p value <0.05 was considered statistically significant.

### Data Availability

All sequence files are available from the NCBI bio-projects (accession number PRJNA648796 and PRJNA648797).

### Machine Learning Model

Random forest (RF) was chosen to build various prediction model (Ob vs lean; ObT2 vs lean; ObT2 vs Ob) using fecal microbes because of its superior performance for classification with binary features. Random Forest⁷ is one of the most popular approaches in metagenomics data analysis to identify the discriminative features and build prediction models. As a widely used ensemble learning algorithm, Random Forest consists of a series of classification and regression trees (CARTs) to form a strong classifier. A subset of data randomly sampled from the original dataset with replacement is known as bootstrap sampling, applying to build the trees. When the training dataset for the current tree is drawn by the bootstrap method,

$$1 {( 1 - \frac{1}{N} )}^{N}$$

observations are left out from the overall dataset. With infinite N, there are 35.11%, 29.25% and 24.36% data not occurred in the training samples called out-of-bag (OOB) observations, which would not be used for constructing the trees. In addition, extra randomness introduced to the random forest as each decision tree splits nodes based on a random subset of features selected from the overall features. The features with the least Gini (Gini are used to evaluate the purity of the node) would be utilized to split the nodes in each iteration to generate the trees. With different subsets of data and features, the algorithm is able to train different trees and obtain the final classification by averaging the result from the tree models. In addition to the prediction model, Random Forest has the capability to assess the importance of variables⁸. The OOB observations are used to estimate the classification error for each tree in the forest. To measure the importance of a given variable, the values of the variable in the OOB data are randomly altered, and then the changed OOB data is used to generate new predictions. The difference of the error rate between the altered and the original OOB observations divided by the standard error is calculated as the importance of a variable. To classify a new sample, the features of the sample passed down to each tree to estimate the probability for classification. The Random Forest used the average probability of all trees to determine the final result of the classification.

The importance value of each species to the classification model was evaluated by recursive feature elimination. According to descending importance value, the selected species were added one by one to the random forest model if its Pearson correlation value with any already existing probe in the model was <0.7. Each time a new feature was added to the model, the performance of the model was re-evaluated using 10-fold cross-validation. These models were compared in terms of binary classifiers with Area Under the Curve (AUC) in Receiver Operating Characteristic (ROC) curves. The final model was chosen when best accuracy and kappa were achieved. These analysis was done using R packages randomForest v4.6-14⁷ and pROC v1.15.3⁹.

### Results

### Clinical Characteristics of Study Subjects

128 obese subjects and 101 lean controls from two regions (Kunming and Hong Kong) in China were included. 131 subjects (78 obese subjects and 53 lean controls) were recruited in Hong Kong (HK) and 98 subjects (50 obese subjects and 48 lean controls) were recruited in Kunming (KM) (Table 6). In the HK cohort, the median age was 47 and 53 years for lean controls and obese subjects respectively. In the KM cohort, the median age was 48.5 and 37 years for lean controls and obesity, respectively. 87.2% of obese subjects in HK had concurrent T2DM (defined as a confirmed diagnosis for at least 3 months) and 12% of obese subjects in KM had T2DM. Other clinical characteristics (gender, alcohol intake and smoking) were comparable between obese subjects and lean controls. On average, 47168171±8610924 clean paired-end reads were obtained from the VLP metagenomic sequencing. In addition, 89829852±13267099 clean paired-end reads were obtained from bulk DNA metagenomic sequencing.

TABLE 6
Demographic and clinical details of obese subjects and lean controls

| Cohort | Factor | Lean | Obese |
|---|---|---|---|
| Hong Kong | Sample size | 53 | 78 |
| (n = 131) | BMI* | 21.2(19.8-22.2) | 33.4(30.7-37.2) |
| | Age* | 47(33-57) | 53(44-60.8) |
| | Male# | 30(56.6%) | 50(64.1%) |
| | Alcohol# | 3(5.7%) | 3(3.9%) |
| | Smoking# | 6(11.3%) | 8(10.3%) |
| | T2DM# | 0 | 68(87.2%) |
| Kunming | Sample size | 48 | 50 |
| (n = 98) | BMI* | 20.8(19.8-21.7) | 29.8(29.1-31.7) |
| | Age* | 48.5(38.8-57) | 37(28.2-52) |
| | Male# | 26(54.2%) | 33(66%) |
| | Alcohol# | 6(12.5%) | 13(26%) |
| | Smoking# | 10(20.8%) | 9(18%) |
| | T2DM# | 0 | 6(12%) |

*median (Interquartile range); # count (percentage); BMI, Body Mass Index (Kg/m2).

### Alterations of Gut Viral Diversity and Taxonomy in Obesity

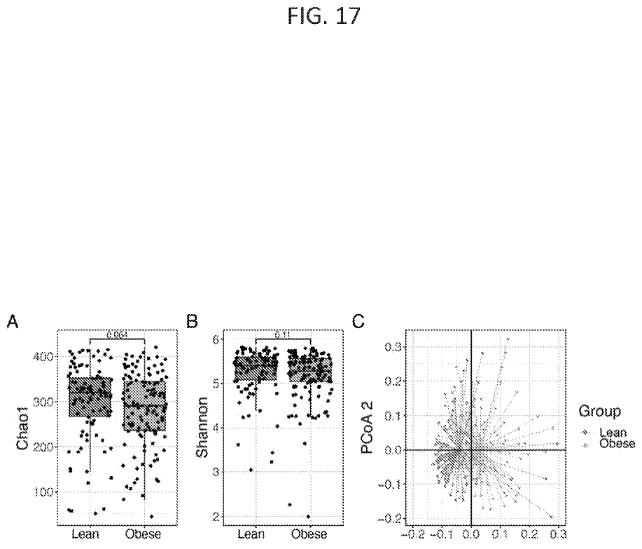

To study the difference of gut virome in obese subjects and lean controls, the inventors first explored viral alpha diversity indices between subjects. At the contig levels, a decreased trend of richness (Chao1) and diversity (Shannon) of gut virome was found in obese subjects compared with lean controls (p=0.064 and p=0.11, respectively, FIG. 17A and FIG. 17B). To exclude confounder effects on the gut virome profiles between obese subjects and lean controls, beta diversity of gut viral contigs was analyzed by adjusting the covariant T2DM. Principal coordinates analysis (PCoA) based on Bray-Curtis distance between individual virome revealed that the gut virome composition of obese subjects and lean controls were separated into two distinct clusters, indicating different gut virome profiles between obese subjects and lean controls (FIG. 17C).

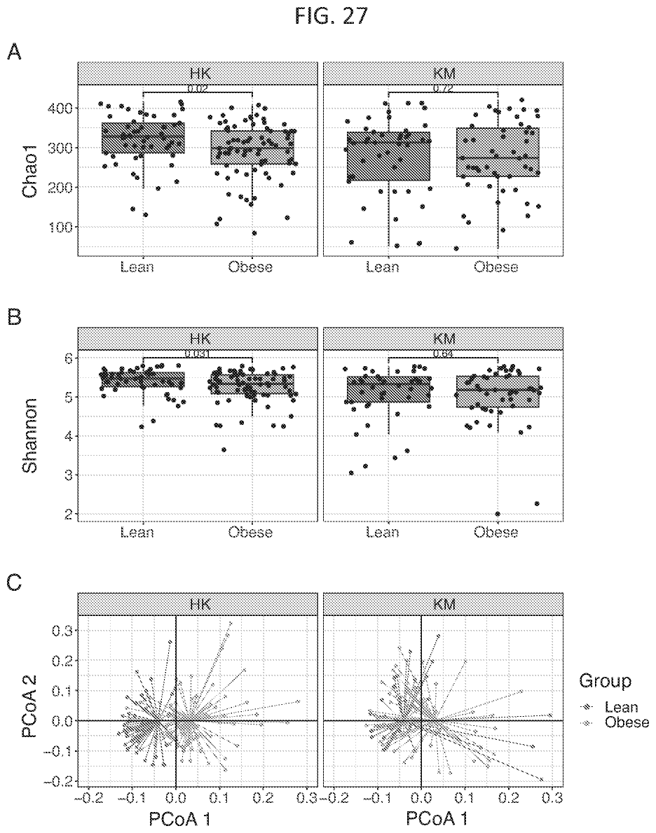

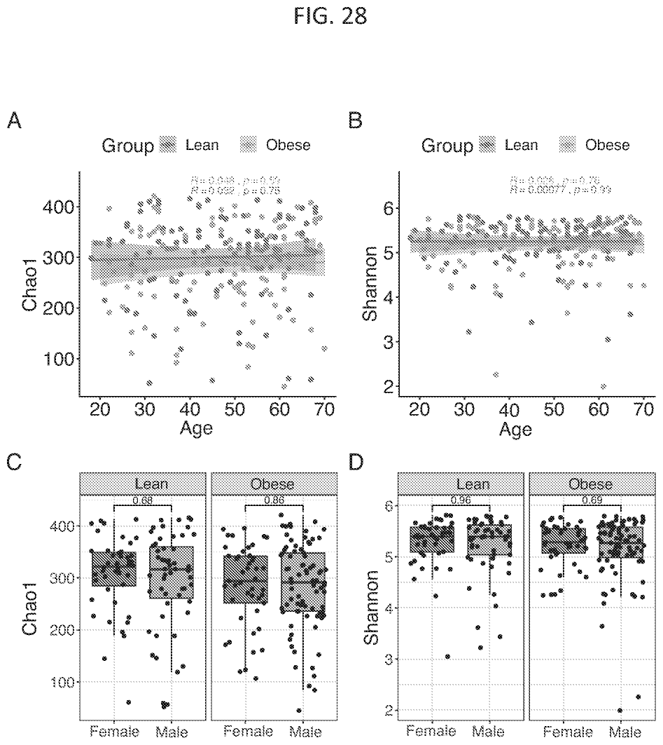

The alpha and beta diversity between cohorts (HK and KM) were further compared to identify the impact of geography on the gut virome. Obese subjects in HK showed a lower alpha diversity (Chao1 and Shannon) compared with lean controls (p<0.05, FIGS. 27A and 27B). In contrast, there was no significant difference in gut viral diversity between obese subjects and lean controls in KM (FIGS. 27A and 27B). PCoA analysis showed a distinct gut viral profile between obese subjects and lean controls in HK and KM cohort (FIG. 27C). These results indicate that although obesity play an important role in gut viral alterations, geographical factor also contributes to variations of the gut virome between cohorts. There was no significant correlation between age and alpha diversity indices (FIGS. 28A and 28B). In addition, no significant difference of alpha diversity between gender in obese subjects and lean controls was observed (FIGS. 28C and 28D). These results indicate that age and gender have little effect on the gut virome profile.

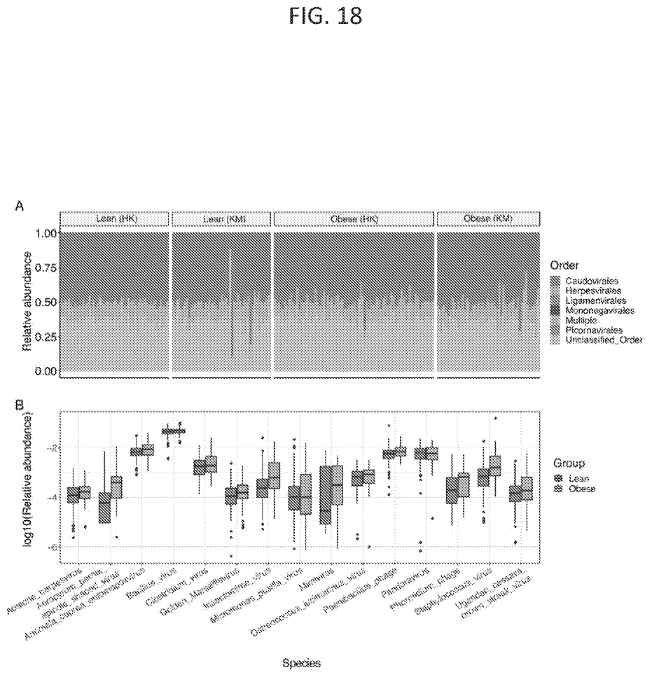

Among the viral orders, Caudovirales which comprise bacteriophages dominated the gut virome in both obese subjects and lean controls (FIG. 18A). Abundance of viral species was correlated with disease phenotype via MaAsLin2 to define gut viral signatures associated with obesity. After correcting for confounders (age, gender, T2DM, cohort, alcohol intake and smoking), fifteen viral species were identified to be significantly enriched in obese subjects compared with lean controls (FIG. 18B; Table 7). Among the differential species, Staphylococcus virus, Bacillus virus and Anomala cuprea entomopoxvirus had the largest effect size. Taken together, these results indicate a different gut viral diversity and taxonomic composition between obese subjects and lean controls.

TABLE 7
Viral species enriched in obese subjects compared with lean controls

| Name | NCBI:txid |
|---|---|
| Abalone herpesvirus | 1821058, 1636535, 1241371, 1003448, 860344 |
| Aeropyrum pernix spindle | 1032473 |
| shaped virus | |
| Anomala cuprea | 62099 |
| entomopoxvirus | |
| Bacillus virus | 396034, 701257, 513550, 1910935, 1273739, 1984785, 10778, |
| | 1918005, 1985182, 1985177, 1985178, 1236573, 2560329, |
| | 1987727, 341938, 1918011, 1985175, 1985176, 1918006, |
| | 1406782, 1910936, 1910937, 1918012, 1918722, 1986015, |
| | 1273740, 1985179, 1273741, 2560330, 1273742, 1084719, |
| | 12345, 2169759, 1406784, 1921017, 1987729, 2560331, 552525, |
| | 1985180, 1918723, 2560332, 1987728, 2560333, 2560334, |
| | 1921018, 10683, 1921185, 1921186, 1921187, 1921188, |
| | 1921189, 1406788, 1918007, 2560335, 2560336, 1921019, 10685, |
| | 66797, 2495532, 1918724, 2560337, 1921710, 1918008, |
| | 1921711, 1921712, 1921713, 57478, 1273744, 1918009, 1985183, |
| | 1922328, 359961, 2169760, 10756, 2511848, 1925723, |
| | 1926346, 2008614, 2008617, 2008616, 2008615 |
| Clostridium virus | 262071, 320122, 559189 |
| Golden Marseillevirus | 1720526 |
| Insectomime virus | 1370065 |
| Micromonas pusilia virus | 1592765, 1592766, 1592767, 1592768, 1592769, 1592770, |
| | 1592771, 1592772, 1592773, 1592774, 755272, 1592775, |
| | 1592776, 1592777, 1592778, 1592779, 1592780, 1592781, |
| | 1592782, 374002, 374001, 374000, 373999, 373997, 373998, |
| | 373996, 374160, 374003 |
| Mimivirus | 315393 |
| Ostreococcus | 880162, 1663208, 1663209, 703950, 703948, 703949, 754062, |
| lucimarinus virus | 703931, 703932, 703933, 703934, 703935, 703936, 754063, |
| | 703937, 703947, 703940, 703941, 703942, 703943, 703944, |
| | 703938, 703939, 754064, 703945, 703946, 754065 |
| Paenibacillus phage | 1296661, 1296662, 1296660, 1296659, 1296658, 2249772 |
| | 2249763, 2070189, 2249764, 2249773, 2249774, 1636254, |
| | 1589750, 2070190, 2249765, 1636255, 2249766, 2249775, |
| | 1589749, 1636256, 1636257, 1702260, 1636258, 2249776, |
| | 2249767, 2249777, 2070191, 2249768, 2070192, 2070193, |
| | 2249769, 2249778, 2070194, 754053, 2070195, 1702259, 1589752, |
| | 2249770, 1636259, 1589754, 1589755, 2070196, 2249771, |
| | 1718161, 2282396, 1636260, 1636261, 2249779, 1636262, |
| | 1636263, 2249780, 666474, 1959007, 1337877, 2530020 |
| Pandoravirus | 2060084 |
| Phormidium_phage | 400567, 440250, 394231, 1391456, 1391455, |
| Staphylococcus virus | 360398, 12360, 186153, 55511, 2734233, 320846, 320840, |
| | 215167, 320837, 204086, 320839, 320847, 320835, 320845, |
| | 2732595, 320834, 320844, 259901, 405947, 53369, 59506, |
| | 320848, 320849, 320842, 2732591, 2500557, 2732596, 2734046, |
| | 2734047, 2562362, 398839, 2732597, 320841, 292029, |
| | 1924732, 2732598, 575610, 1197952, 1197953, 575608, 1980931, |
| | 2508849, 1924729, 221915, 1924733, 1924731, 399185, |
| | 2732601, 2732599, 2734234, 1857890, 1980962, 2732602, |
| | 1980964, 1924734, 1204543, 1924730, 2732603, 872294, |
| | 2732604, 1980930, 1922247, 2732605, 2508850, 2508851, |
| | 2560781, 1922246, 2734053, 2732592, 2042206, 55510, 320850, |
| | 2736346, 2736347, 186152, 2732600, 106284, 326036, 326037, |
| | 379501, 487152, 387907, 387908, 387910, 130478 |
| Ugandan cassava brown | 946046, 980531, 980521, 980522, 980525, 980526, 980527, |
| streak virus | 980528, 980529, 980523, 980530, 980524, 980540, 980534, |
| | 980532, 980533, 980539, 931971, 980537, 980535, 980538, |
| | 980536 |

As such, gut virome richness (Chao1) and diversity (Shannon) can be used alone or in combination with other factors such as geography, urbanicity as an indicator for risk of obesity. Further, viral species listed in Table 7 can be used either alone or in different combinations to determine the risk of obesity. For example, the relative abundance can be determined using as a panel of qPCR primer or by metagenomics sequencing, and such relative abundance can be compared to a reference population to calculate the risk.

### T2DM Contributed to Gut Virome Alterations in Obesity

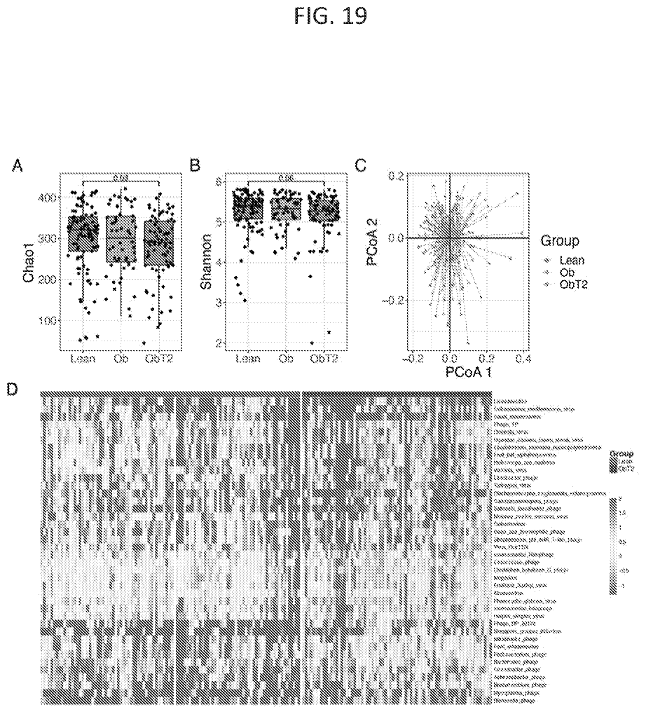

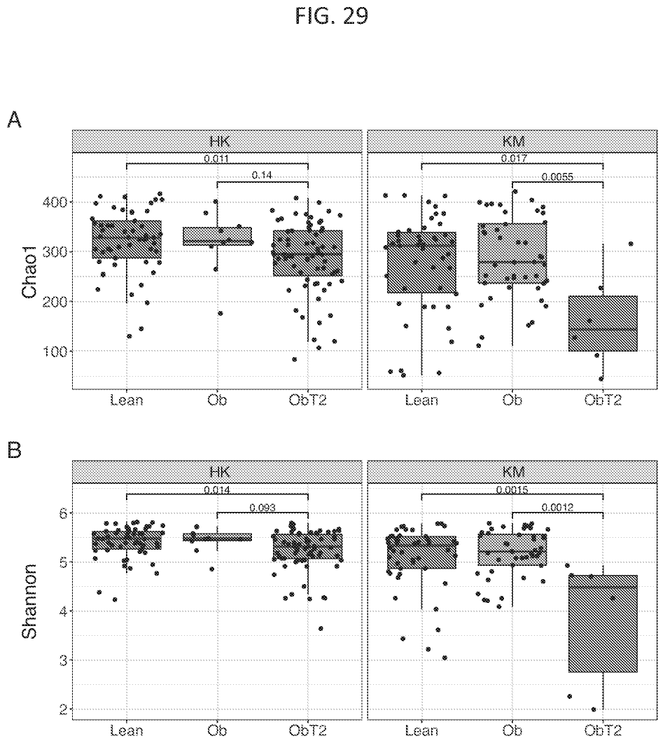

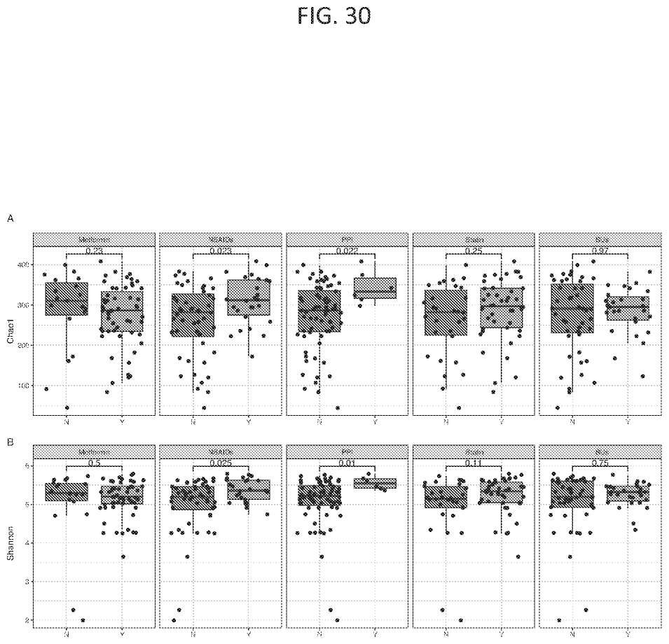

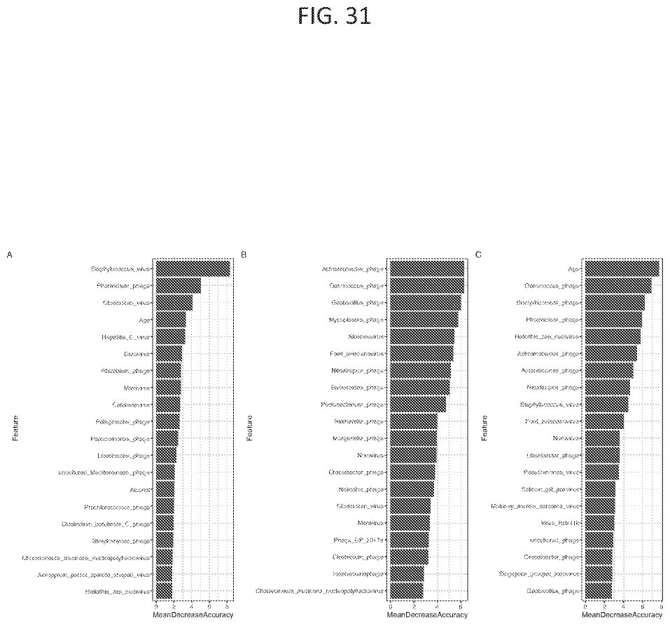

To explore whether T2DM affects the gut virome in obesity, alpha diversity was compared among obese subjects without T2DM (Ob), obese subjects with T2DM (ObT2), and lean controls. Though a decreased viral Chao1 richness and Shannon diversity were observed in Ob compared with lean controls, the decrease was greater in ObT2 compared with lean controls (FIG. 19A and FIG. 19B). ObT2 showed a significantly decreased viral richness compared with lean controls (p=0.03, FIG. 19A). Similarly, a reduced trend of viral diversity was observed in ObT2 compared with lean controls (p=0.06, FIG. 19B). A decreased viral richness and diversity was also observed in ObT2 compared with lean controls both in HK and KM cohorts, indicating that ObT2 exhibited unique gut viral profiles distinct from lean controls in both cohorts (FIGS. 29A and 29B). PCoA analysis revealed that the gut virome composition of ObT2 separated from Ob and lean controls, indicating significant alterations for gut virome profile in ObT2 compared with Ob and lean controls (FIG. 19C).

The associations between gut viral diversity and common medications including Metformin, Sulfonylureas (SUs), Statin, Proton-pump inhibitors (PPIs) and Non-Steroidal Anti-Inflammatory Drugs (NASIDs) were next explored (FIG. 30). In the ObT2, NSAIDS and PPI users had a higher viral alpha diversity than non-medication users (all p value <0.05), but there was no significant difference in viral diversity in subjects with Metformin, SUs and Statin and those without suggesting that antidiabetic and anti-lipid agents did not have a major impact on the gut virome.

To further explore viral species associated with subjects who had concurrent obesity and T2DM, 40 viral species were found to significantly associate with ObT2 subjects compared with lean controls after adjusting for age, gender, cohort, alcohol intake and smoking (FIG. 19D). Among them, 11 viral species (Phage DP 2017a, Croceibacter phage, Bacteroides phage, etc.) were increased in ObT2 (Table 8) whereas 29 viral species (Klosneuvirus, Oenococcus phage, Clostridium botulinum C phage, etc.) were depleted in ObT2 compared with lean controls (Table 9). Among the differential viral species compared with lean controls, only Ugandan cassava brown streak virus was found to be highly present in both ObT2 and obese subjects. These results indicate different gut viral taxonomic signature between Ob, ObT2, and lean controls at the species level. Overall, T2DM was associated with a more perturbed gut viral dysbiosis in obesity.

TABLE 8
Viral species enriched in ObT2 subjects compared with lean controls

| Viral Species | NCBI:txid |
|---|---|
| Achromobacter phage | 1589747, 2723726, 2723727, 2292880, 1664247, 1664246, 1610509, 2591403, |
| | 1589746, 1589748, 1416008, 1416009, 2723728, 2591054, 2591053, 2591039, |
| | 2591040, 2591041, 2591042, 2591043, 2591044, 2591045, 2591046, 2591047, |
| | 2591048, 2591049, 2591050, 2591051, 2591052 |
| Bacteroides phage | 2710493, 2710494, 2304657, 1105171, 99179, 2596712, 2710495, 2710496, |
| | 2710497, 2710498, 2710499, 2710500, 2710501, 2710502, 2710503, 2303977, |
| | 2710504, 2710505, 2710506, 2710507, 2710508, 2710509, 2710510, 2710511, |
| | 2710512, 2710513, 2710514, 2710515, 2710516, 2710517, 2710518, 2710519, |
| | 2301731, 2306278, 2486354, 2486353, 2733869 |
| Bradyrhizobium phage | 1983459 |
| Croceibacter phage | 1327037,1176422 |
| Fowl aviadenovirus | 586029, 10553, 10547, 172857, 172859, 172860, 130663, 172861, 172862, |
| | 172863, 66295, 172864, 190061, 190062, 190063, 190064, 190065, 1972696 |
| Mycoplasma phage | 35238, 280702, 75590, |
| Nitratiruptor phage | 1230469 |
| Pectobacterium phage | 2053078, 1907173, 2488835, 2500577, 2500578, 2489617, 1792242, 2652426, |
| | 2652427, 2489618, 2489619, 2489620, 2489621, 2489622, 2489623, 2489624, |
| | 2489625, 2041488, 2041489, 2041490, 2041491, 2041492, 2489626, 2320194, |
| | 2163634, 2489627, 2489628, 2489629, 2320195, 2662283, 2686474, 2662284, |
| | 2686475, 2608319, 2608298, 2608323, 2320196, 1204539, 2163635, 2320197, |
| | 2489630, 2489631, 2489632, 2489633, 1399915, 1965269, 1217810, 1916414, |
| | 1873958, 1897743, 1932882, 1916101, 1927014, 1873959, 1932883, 1685500, |
| | 2153295, 1961914, 1654601, 1211386, 2489634, 2500576, 2320198, 2652428, |
| | 2652429, 1127516, 2489635, 2489636, 1965354, 1116482, 1958916, 1958917, |
| | 1958918, 1958919, 1983582 |
| Phage DP 2017a | 1955560 |
| Riemerella phage | 936152 |
| Singapore grouper | 262968 |
| iridovirus | |

TABLE 9
Viral species depleted in ObT2 subjects compared with lean controls

| Viral Species | NCBI:txid |
|---|---|
| Chlorella virus | 10507 |
| Choristoneura murinana | 1987479 |
| nucleopolyhedrovirus | |
| Clostridium botulinum C | 12336 |
| phage | |
| Deep sea thermophilic phage | 749413 |
| Diachasmimorpha | 109981 |
| longicaudata entomopoxvirus | |
| Emiliania huxleyi virus | 181208 |
| Environmental halophage | 436674 |
| Environmental Halophage | 1168827 |
| Fruit bat alphaherpesvirus | 1343901 |
| Gokushovirus | 1758150, 2507516, 2073143, 2723245, 2723244 |
| Helicoverpa zea nudivirus | 1128424 |
| Herpes simplex virus | 126283, 10298, 10299, 10300, 10301, 10302, 37106, 10304, 10303, 10305, |
| | 10306, 37107, 10307, 10308, 36345, 36346, 946522, 10309, 10310, 10368, |
| | 10372, 10312, 10313, 103921, 10314, 10315, 10316, 10369, 10370, 36351, |
| | 57278, 57279 |
| Klosneuvirus | 1977639 |
| Lausannevirus | 999883 |
| Liberibacter phage | 1072683, 1903185, 2212825, 2212812, 2212813, 2212814, 2212815, |
| | 2212824, 2212816, 2212817, 1965455, 2212818, 2212819, 2212820, |
| | 2212821, 2212822, 2212823, 2212826, 2212827, 941969, 941970, 1903184 |
| Megavirus | 1643304, 1643300, 1643303, 1643305, 1242814, 1686594, 1643301, |
| | 1128139, 1094892, 1128140, 1128141, 1128135, 1686595, 1235314, |
| | 1242815, 1686770, 1128142, 1643508, 2751880, 1686596, 1686597, |
| | 1643296, 1128143, 1686598, 1643298, 2109586, 2711275 |
| Moloney murine sarcoma | 11809, 31691, 11811, 11810 |
| virus | |
| Oenococcus phage | 2036701, 1432848, 1432847, 2201414, 1435411, 1885654, 1885653, |
| | 1885652, 1885651, 1885650, 1885649, 1885648, 1885647, 1885646, |
| | 1885645, 1885644, 1885643, 1885642, 1885641, 1885640, 1303346 |
| Ostreococcus mediterraneus | 1663210, 2726183 |
| virus | |
| Phaeocystis globosa virus | 251749 |
| Phage TP | 282785 |
| Saccharomonospora phage | 182851 |
| Salisaeta icosahedral phage | 1183239 |
| Saudi moumouvirus | 1956188 |
| Streptococcus phi m46 1-like | 1028788 |

phage

| | |
|---|---|
| Turkeypox virus | 336486 |
| Ugandan cassava brown | 946046, 980531, 980521, 980522, 980525, 980526, 980527, 980528, |
| streak virus | 980529, 980523, 980530, 980524, 980540, 980534, 980532, 980533, |
| | 980539, 931971, 980537, 980535, 980538, 980536 |
| Vaccinia virus | 332193, 10245, 126794, 10249, 10250, 502057, 10251, 31531, 10248, |
| | 10246, 130665, 10252, 691321, 301352, 10253, 1651169, 10247, 10254, |
| | 696871, 130666 |
| Virus Rctr197k | 1924548 |

As such, viral species listed in Table 8 and Table 9 can be used either alone or in different combinations to predict the risk of obesity with type 2 diabetes. In particular, Ugandan cassava brown streak virus can be used as a marker to predict risk of obesity. For example, the relative abundance can be determined using as a panel of qPCR primer or by metagenomics sequencing to calculate the predicted severity.

Furthermore, viral species listed Table 9 can be administered to subjects with obesity or type 2 diabetes for reduction of body weight and control of type 2 diabetes.

### Machine Learning Model to Predict Risk of T2DM

### Model 1: Obese (Ob) Vs Lean Control (Lean)

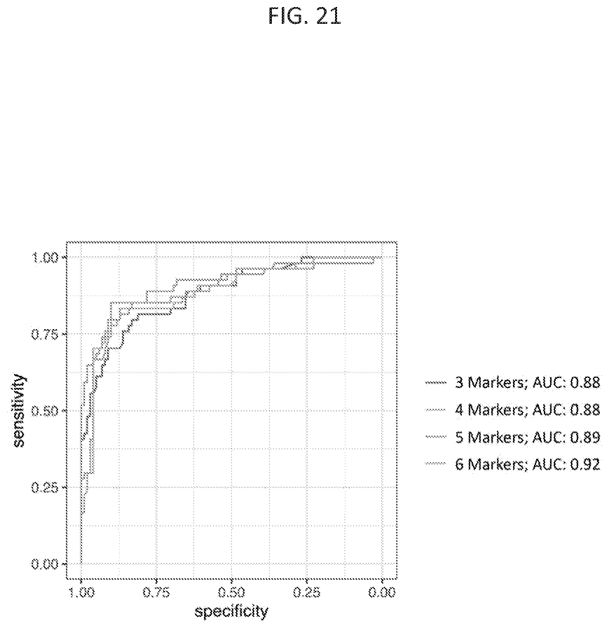

A total of 54 Ob subjects and 101 lean controls were included as the discovery cohort for modelling. Five viral markers, including Staphylococcus virus, Phormidium phage, Clostridium virus, Hepatitis C virus, Catovirus, and age were included in the machine learning model (Table 10). The final models using these 6 markers has an Area Under the Curve (AUC) in Receiver Operating Characteristic (ROC) curves of 91.51% (FIG. 21).

TABLE 10
Viral species included in the machine learning model for prediction of obesity

| Viral Species | NCBI:txid |
|---|---|
| Staphylococcus virus | 360398, 12360, 186153, 55511, 2734233, 320846, 320840, 215167, 320837, 204086, |
| | 320839, 320847, 320835, 320845, 2732595, 320834, 320844, 259901, 405947, 53369, 59506, |
| | 320848, 320849, 320842, 2732591, 2500557, 2732596, 2734046, 2734047, 2562362, 398839, |
| | 2732597, 320841, 292029, 1924732, 2732598, 575610, 1197952, 1197953, 575608, |
| | 1980931, 2508849, 1924729, 221915, 1924733, 1924731, 399185, 2732601, 2732599, |
| | 2734234, 1857890, 1980962, 2732602, 1980964, 1924734, 1204543, 1924730, 2732603, |
| | 872294, 2732604, 1980930, 1922247, 2732605, 2508850, 2508851, 2560781, 1922246, |
| | 2734053, 2732592, 2042206, 55510, 320850, 2736346, 2736347, 186152, 2732600, 106284, |
| | 326036, 326037, 379501, 487152, 387907, 387908, 387910, 130478 |
| Phormidium phage | 400567, 440250, 394231, 1391456, 1391455 |
| Clostridiu virus | 262071, 320122, 559189 |
| Hepatitis C virus | 11103 |
| Catovirus | 1977635, 1977631 |

TABLE 11
Relative abundance of vial species listed in Table 13 and age of obese (Ob) subjects and
lean control (lean)

| Group | Staphylococcus virus | Phormidium phage | Costridium virus | age | Hepatitis C virus | Catovirus |
|---|---|---|---|---|---|---|
| Lean | 6.21E−04 | 0 | 0.005948 | 28 | 5.87E−05 | 0.029335 |
| Lean | 2.48E−05 | 0 | 0.002368 | 32 | 0 | 0.032699 |
| Lean | 2.56E−04 | 0.001933 | 4.01E−04 | 68 | 7.77E−05 | 0.033345 |
| Lean | 0.001303 | 5.96E−04 | 0.001055 | 68 | 6.30E−05 | 0.051852 |
| Lean | 1.32E−04 | 0 | 0.00149 | 36 | 0 | 0.03316 |
| Lean | 4.10E−04 | 0 | 0.003241 | 40 | 0.004351 | 0.034886 |
| Lean | 2.78E−04 | 0 | 0.002496 | 33 | 6.54E−05 | 0.052365 |
| Lean | 7.34E−04 | 0.001458 | 0.005713 | 52 | 0 | 0.023672 |
| Ob | 0.001546 | 1.06E−04 | 0.001632 | 45 | 3.01E−06 | 0.02479 |
| Ob | 6.78E−04 | 0 | 0.001126 | 63 | 0 | 0.010507 |
| Ob | 0.002111 | 0 | 9.10E−04 | 29 | 0.003158 | 0.030216 |
| Lean | 5.95E−04 | 0 | 8.64E−04 | 42 | 1.55E−04 | 0.014543 |
| Lean | 7.13E−04 | 4.12E−05 | 0.003641 | 29 | 2.58E−05 | 0.015157 |
| Ob | 0.001556 | 0 | 0.001152 | 52 | 0 | 0.030068 |
| Ob | 6.05 E−04 | 0 | 0.006743 | 25 | 0 | 0.022372 |
| Ob | 0.001529 | 0 | 0.006676 | 29 | 0 | 0.025146 |
| Ob | 0.002294 | 0 | 0.003266 | 47 | 0 | 0.026386 |
| Ob | 0.00434 | 0 | 2.82E−04 | 22 | 0 | 0.03235 |
| Ob | 9.37E−04 | 3.13E−04 | 0.002289 | 20 | 0 | 0.014212 |
| Ob | 3.87E−04 | 0 | 0.001305 | 53 | 0 | 0.00627 |
| Ob | 8.11 E−04 | 9.49E−04 | 0.002014 | 22 | 0 | 0.014801 |
| Ob | 0.018848 | 0 | 7.50E−04 | 27 | 0 | 0.013434 |
| Ob | 0.001341 | 0.001022 | 7.45E−04 | 33 | 1.10E−05 | 0.017425 |
| Ob | 0.001183 | 0 | 0.001859 | 22 | 0 | 0.029945 |
| Ob | 0.001168 | 0.001488 | 0.001284 | 51 | 0.001454 | 0.019601 |
| Ob | 1.11E−04 | 7.76E−04 | 0.016158 | 46 | 0 | 0.05085 |
| Ob | 0.00211 | 0 | 9.94E−04 | 52 | 0 | 0.028407 |
| Ob | 0.001408 | 1.52E−04 | 9.41E−04 | 61 | 0.002706 | 0.020631 |
| Ob | 4.19 E−04 | 1.66E−04 | 0.0021 | 37 | 0 | 0.01225 |
| Ob | 0.001864 | 0 | 0.005847 | 29 | 7.80E−05 | 0.048852 |
| Ob | 9.19E−04 | 0 | 0.007584 | 66 | 2.32E−04 | 0.030828 |
| Lean | 0 | 0 | 1.41E−04 | 51 | 0 | 0.030054 |
| Lean | 2.27E−04 | 0 | 0.004189 | 67 | 0 | 0.026095 |
| Lean | 0.005055 | 0 | 0.00148 | 38 | 0 | 0.024364 |
| Lean | 5.45E−04 | 0 | 0.00457 | 44 | 1.43E−05 | 0.026927 |
| Lean | 1.66E−04 | 0.00161 | 0.006335 | 66 | 6.43E−05 | 0.012674 |
| Lean | 0 | 0 | 0.003059 | 38 | 0 | 0.04624 |
| Lean | 0.018097 | 0 | 0.002992 | 27 | 3.02E−05 | 0.025492 |
| Lean | 1.95E−04 | 0 | 9.73E−04 | 25 | 0 | 0.026459 |
| Lean | 0 | 0 | 0.00179 | 41 | 0 | 0.024466 |
| Lean | 8.24E−04 | 0.005501 | 4.55E−04 | 47 | 0 | 0.020003 |
| Lean | 1.40E−04 | 3.36E−05 | 0.001278 | 39 | 3.02E−05 | 0.035033 |
| Lean | 0.001017 | 1.62E−04 | 7.45E−04 | 22 | 0.001674 | 0.033753 |
| Lean | 0.001392 | 0 | 0.006749 | 53 | 0.001079 | 0.027511 |
| Lean | 0.002544 | 0 | 7.58E−04 | 21 | 0 | 0.026756 |
| Lean | 0.001599 | 3.02E−04 | 0.001886 | 44 | 3.90E−06 | 0.038925 |
| Lean | 3.73E−05 | 0 | 4.49E−04 | 62 | 0 | 0.005166 |
| Lean | 0.001969 | 5.72E−05 | 0.002205 | 62 | 0.003449 | 0.041215 |
| Lean | 9.79E−06 | 0 | 0 | 67 | 0 | 0.020989 |
| Lean | 0.00285 | 0 | 0.002397 | 44 | 1.04E−04 | 0.009984 |
| Lean | 0.007308 | 0 | 7.32E−04 | 26 | 0 | 0.035633 |
| Lean | 0 | 0 | 3.49E−04 | 31 | 0 | 8.34E−04 |
| Lean | 1.14E−05 | 3.38E−05 | 0.001298 | 67 | 9.12E−04 | 0.031348 |
| Lean | 4.59E−04 | 2.62E−05 | 4.12E−04 | 42 | 1.19E−05 | 0.026506 |
| Lean | 1.79E−04 | 0 | 6.43E−04 | 46 | 0 | 0.024674 |
| Lean | 0.00395 | 0 | 9.93E−04 | 37 | 0 | 0.026912 |
| Lean | 6.45 E−04 | 0 | 0.007022 | 60 | 0 | 0.029398 |
| Lean | 0.001396 | 1.11E−04 | 4.25E−04 | 47 | 3.77E−05 | 0.016399 |
| Lean | 6.14E−04 | 1.30E−05 | 8.31 E−04 | 55 | 3.88E−05 | 0.038126 |
| Lean | 4.92E−04 | 2.41E−04 | 0.00496 | 60 | 1.27E−05 | 0.048877 |
| Lean | 7.38E−04 | 0 | 0.005708 | 62 | 0 | 0.013032 |
| Lean | 0.002131 | 0 | 7.77E−04 | 43 | 3.23E−05 | 0.028016 |
| Lean | 1.62E−04 | 0 | 0.005666 | 62 | 0 | 0.049429 |
| Lean | 8.79E−04 | 3.35E−04 | 0.001374 | 59 | 0 | 0.024066 |
| Lean | 0.002266 | 0.001807 | 5.45E−04 | 60 | 2.91E−05 | 0.033033 |
| Lean | 0.001214 | 1.78E−04 | 0.002773 | 65 | 6.87E−06 | 0.024187 |
| Lean | 0.001221 | 5.15E−05 | 0.001728 | 51 | 6.84E−05 | 0.025296 |
| Lean | 4.58E−04 | 1.92E−04 | 0.001949 | 31 | 0.002418 | 0.029075 |
| Lean | 3.47E−04 | 0 | 0.00209 | 53 | 1.14E−05 | 0.026249 |
| Lean | 0.002758 | 0 | 6.57E−04 | 31 | 0 | 0.038326 |
| Lean | 9.39E−05 | 0 | 0.005701 | 48 | 3.03E−05 | 0.042563 |
| Lean | 5.18E−04 | 0 | 0.004292 | 55 | 1.66E−04 | 0.052959 |
| Lean | 0.0014 | 0 | 2.46E−04 | 36 | 2.21E−06 | 0.0164 |
| Lean | 7.91E−04 | 0.001455 | 0.002255 | 34 | 1.32E−04 | 0.029501 |
| Lean | 0.001131 | 0 | 7.20E−04 | 50 | 0 | 0.021854 |
| Lean | 7.22E−05 | 0 | 8.80E−04 | 35 | 0 | 0.023162 |
| Lean | 0.001036 | 1.28E−04 | 0.002112 | 26 | 4.97E−05 | 0.025395 |
| Lean | 1.27E−04 | 0 | 8.44E−04 | 47 | 0 | 0.03368 |
| Lean | 7.87E−04 | 0 | 0.002597 | 39 | 0 | 0.028225 |
| Lean | 5.50E−04 | 7.58E−05 | 0.002055 | 33 | 1.27E−04 | 0.021027 |
| Lean | 5.09E−04 | 0 | 0.001047 | 32 | 0 | 0.032201 |
| Lean | 1.45E−04 | 0 | 0.012521 | 51 | 0 | 0.017634 |
| Lean | 0.002599 | 0 | 0.001572 | 22 | 0 | 0.024597 |
| Lean | 2.64E−04 | 0 | 0.001247 | 31 | 2.61E−05 | 0.014875 |
| Lean | 3.37E−04 | 5.60E−05 | 0.005855 | 19 | 1.97E−05 | 0.026275 |
| Lean | 9.43E−04 | 0 | 0.002564 | 18 | 0 | 0.021251 |
| Lean | 3.47E−04 | 0 | 6.96E−04 | 37 | 1.65E−05 | 0.020037 |
| Ob | 0.001313 | 4.31E−04 | 0.007352 | 53 | 0.005308 | 0.047924 |
| Ob | 8.19E−04 | 1.01E−04 | 0.001849 | 57 | 0 | 0.026161 |
| Ob | 1.08E−04 | 5.44E−04 | 0.00134 | 34 | 0 | 0.029482 |
| Ob | 3.41E−04 | 0 | 6.08E−04 | 37 | 0.003792 | 0.036274 |
| Ob | 8.95E−04 | 0 | 6.21E−04 | 32 | 0 | 0.012512 |
| Ob | 0.001333 | 6.62E−04 | 0.006101 | 27 | 0 | 0.030985 |
| Ob | 2.44E−04 | 9.83E−05 | 0.003151 | 50 | 2.37E−05 | 0.020424 |
| Ob | 0.004855 | 0.005181 | 0.002815 | 29 | 1.72E−06 | 0.026896 |
| Ob | 0.151513 | 6.33E−04 | 0.001817 | 23 | 0 | 0.027432 |
| Ob | 0.008583 | 0 | 7.84E−04 | 27 | 0 | 0.008296 |
| Ob | 0.004488 | 0.001695 | 0.004697 | 29 | 1.47E−05 | 0.025986 |
| Ob | 0.00623 | 0 | 0.02542 | 30 | 0 | 0.022349 |
| Ob | 0.005652 | 0 | 0.006687 | 21 | 0 | 0.014061 |
| Ob | 0.004695 | 0.002092 | 0.003087 | 56 | 7.60E−06 | 0.018955 |
| Ob | 5.13E−04 | 0 | 0.001871 | 50 | 0 | 0.0433 |
| Ob | 0.004872 | 0.002041 | 0.002793 | 27 | 0 | 0.02379 |
| Ob | 0.001719 | 8.17E−04 | 0.000577 | 27 | 6.39E−04 | 0.014872 |
| Ob | 6.28E−04 | 0 | 0.006495 | 42 | 0.00264 | 0.033006 |
| Ob | 0.002989 | 1.57E−05 | 0.001047 | 32 | 1.53E−05 | 0.014925 |
| Ob | 0.001876 | 0 | 0.001241 | 31 | 2.79E−04 | 0.02062 |
| Ob | 0.006665 | 1.75E−05 | 6.11E−04 | 40 | 0 | 0.005906 |
| Ob | 0.007194 | 0 | 0.001556 | 69 | 2.08E−05 | 0.01976 |
| Ob | 4.55E−04 | 0 | 0.008637 | 51 | 0 | 0.020866 |
| Ob | 4.86E−04 | 8.86E−04 | 0.010499 | 26 | 0 | 0.04836 |
| Ob | 0.003521 | 0 | 0.002759 | 37 | 0 | 0.015875 |
| Ob | 6.04E−04 | 6.32E−05 | 0.002035 | 39 | 0.002155 | 0.033567 |
| Ob | 0.0058 | 3.00E−05 | 0.012033 | 28 | 0 | 0.026641 |
| Ob | 0.009639 | 0 | 0.00143 | 41 | 0 | 0.03939 |
| Ob | 0.016706 | 0 | 9.40E−04 | 52 | 3.33E−04 | 0.009904 |
| Ob | 0.001919 | 5.27E−05 | 0.002087 | 55 | 3.74E−05 | 0.010425 |
| Ob | 0.001846 | 6.56E−04 | 0.001443 | 32 | 4.34E−04 | 0.026081 |
| Ob | 0.00336 | 0 | 3.97E−04 | 33 | 4.48E−06 | 0.02144 |
| Ob | 2.34E−04 | 8.25E−04 | 6.23E−04 | 61 | 7.59E−05 | 0.021981 |
| Lean | 5.47E−04 | 0 | 0.004019 | 55 | 0 | 0.021615 |
| Lean | 1.09E−04 | 0 | 7.42E−04 | 56 | 0 | 0.032477 |
| Lean | 7.76E−04 | 0 | 0.002045 | 53 | 0 | 0.021443 |
| Lean | 9.79E−04 | 0 | 0.001034 | 33 | 0 | 0.041047 |
| Lean | 5.31E−04 | 0 | 0.002152 | 48 | 0 | 0.039607 |
| Lean | 0.001754 | 0 | 0.001394 | 60 | 0 | 0.016276 |
| Lean | 0.006561 | 0 | 0.001258 | 49 | 0 | 0.028331 |
| Lean | 0.001187 | 0 | 0.003325 | 38 | 4.38E−05 | 0.029655 |
| Lean | 9.19E−04 | 0 | 0.002366 | 57 | 0 | 0.022977 |
| Lean | 4.96E−04 | 0 | 0.004904 | 55 | 1.25E−05 | 0.025503 |
| Lean | 9.83E−06 | 0 | 0.001908 | 28 | 0 | 0.037191 |
| Lean | 0 | 0 | 0.002239 | 40 | 0 | 0.026268 |
| Lean | 7.13E−04 | 0 | 6.40E−04 | 68 | 0 | 0.0168 |
| Lean | 3.22E−04 | 2.41E−04 | 0.001179 | 65 | 0 | 0.02342 |
| Lean | 6.23E−05 | 1.09E−04 | 4.59 E−04 | 53 | 0 | 0.02796 |
| Lean | 1.44E−05 | 0 | 1.29E−04 | 45 | 0 | 5.89E−04 |
| Lean | 0 | 0 | 0 | 56 | 0 | 0.002771 |
| Lean | 0.001712 | 0 | 0.002417 | 29 | 9.57E−05 | 0.029518 |
| Lean | 6.28E−04 | 7.83E−04 | 0.001902 | 50 | 1.18E−05 | 0.039551 |
| Lean | 0.001382 | 4.42 E−04 | 0.003956 | 48 | 0 | 0.024446 |
| Lean | 0 | 0 | 0 | 63 | 0 | 0 |
| Lean | 4.13E−05 | 7.43E−06 | 0.001604 | 59 | 7.11E−06 | 0.022832 |
| Lean | 2.14E−04 | 2.35E−04 | 0.007805 | 42 | 0.0014 | 0.027204 |
| Lean | 0.01046 | 0 | 6.36E−04 | 56 | 0 | 0.017992 |
| Lean | 0.000347 | 0 | 6.93E−04 | 57 | 0 | 0.026813 |
| Lean | 0.001064 | 0 | 0.002986 | 26 | 1.03E−05 | 0.056305 |
| Lean | 0.001075 | 7.15E−04 | 0.001199 | 58 | 0.010884 | 0.050082 |
| Lean | 0.00191 | 0 | 0.002232 | 66 | 0 | 0.028495 |
| Lean | 4.22E−04 | 1.99E−04 | 0.005225 | 59 | 8.57E−05 | 0.021384 |
| Lean | 0.00136 | 0 | 0.003265 | 56 | 2.05E−05 | 0.031192 |
| Lean | 0.002242 | 0 | 7.75E−04 | 56 | 0 | 0.024712 |
| Lean | 0.007695 | 8.02E−05 | 0.001729 | 57 | 1.74E−04 | 0.025373 |
| Lean | 0.001226 | 0 | 0.001113 | 62 | 0 | 0.018139 |
| Lean | 3.11E−04 | 0 | 0.004017 | 53 | 0 | 0.01883 |
| Lean | 6.98E−04 | 0 | 6.58E−04 | 53 | 2.76E−05 | 0.04949 |

As such, to determine the risk of obesity in a subject, the following steps will be carried out:
1. Obtain a set of training data by determine the age of subjects and relative abundance of species selected from Table 10* in a cohort of obese subjects and lean controls.
2. Determine the relative abundance of these species in the subject whose risk of obesity is to be determined.
3. Compare the relative abundance of these species in the subject with the training data using random forest model.
4. Decision trees will be generated by random forest from the training data. The relative abundances will be run down the decision trees and generate a risk score. If more than 50% trees in the model consider the subject obese, the outcome will be “subject being tested is deemed to be at an increased risk for obesity”. If less than 50% trees in the model consider the subject as lean, the outcome will be “subject being tested is deemed to be at low risk for obesity”.
   *species selected from Table 10 comprise of
   1. Staphylococcus virus, Phormidium phage, Costridium virus (top 3 markers; AUC: 88.19%; FIG. 21);
   2. Staphylococcus virus, Phormidium phage, Costridium virus, age (top 4 markers; AUC: 88.33%; FIG. 21);
   3. Staphylococcus virus, Phormidium phage, Costridium virus, age, Hepatitis C virus (top 5 markers; AUC: 88.89%; FIG. 21); or
   4. Staphylococcus virus, Phormidium phage, Costridium virus, age, Hepatitis C virus, Catovirus (all 6 markers; AUC: 91.51%; FIG. 21)

### EXAMPLE

The relative abundance of 6 species listed in Table 10 from Lean (n=101) and Ob (n=54) was determined by metagenomics sequencing and taxonomy assigned as described in methods (relative abundance listed in Table 10). Decision trees were generated by random forest from data in Table 10 with parameter: trees=100, mtry=4.

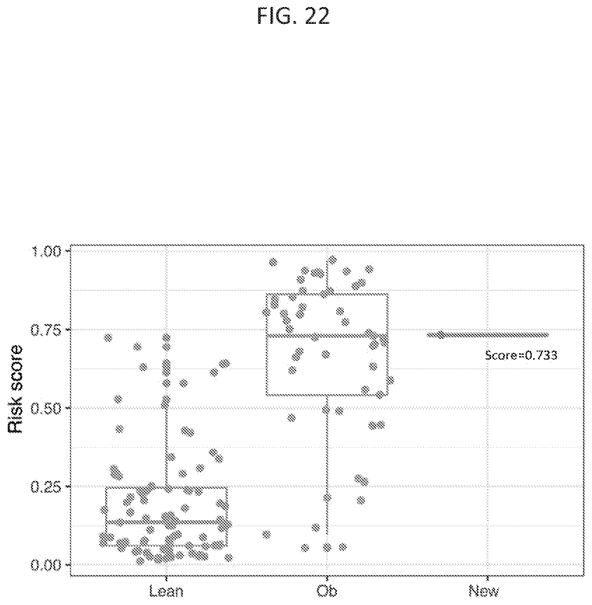

The likelihood of having obesity in a 34-year-old female subject (FB004) was determined. The relative abundance of the 5 species listed in Table 10 in fecal sample of this subject was determined by metagenomics sequencing and taxonomy assigned as described in method. Relative abundance of the 5 species in this subject is shown in Table 12. The relative abundances were run down the decision trees and a risk score was generated using relative abundances listed in Table 11 as training data. The score of the subject was 0.733 (FIG. 22), and therefore the subject was deemed to a have higher risk for obesity. This subject had BMI 41.5 (obese).

TABLE 12
Relative abundance of 5 vial species listed in Table 13 and age
of a subject whose risk of obesity is to be determined

| Staphylococcus | Phormidium | Clostridium | | Hepatitis | |
|---|---|---|---|---|---|
| virus | phage | virus | age | C virus | Catovirus |
| 9.48E−04 | 0 | 0.006826 | 34 | 0.00204 | 0.039188 |

Model 2: Obese with Type 2 Diabetes (ObT2) Vs Lean Control

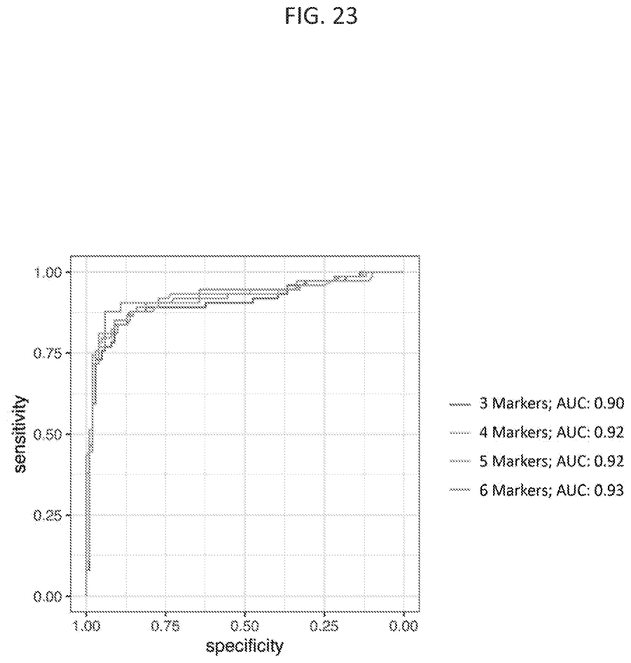

A total of 74 ObT2 subjects and 101 lean controls were included as the discovery cohort for modelling. Six viral markers, including Achromobacter phage, Oenococcus phag, Geobacillus phage, Mycoplasma phage, Klosneuvirus, and Fowl aviadenovirus were included in the machine learning model (Table 13). The final models using these 6 markers has an Area Under the Curve (AUC) in Receiver Operating Characteristic (ROC) curves of 93.2% (FIG. 23).

TABLE 13
Viral species included in the machine learning model for prediction of obesity
with type2 diabetes (ObT2)

| Viral Species | NCBI:txid |
|---|---|
| Achromobacter phage | 1589747, 2723726, 2723727, 2292880, 1664247, 1664246, 1610509, 2591403, |
| | 1589746, 1589748, 1416008, 1416009, 2723728, 2591054, 2591053, 2591039, |
| | 2591040, 2591041, 2591042, 2591043, 2591044, 2591045, 2591046, 2591047, |
| | 2591048, 2591049, 2591050, 2591051, 2591052 |
| Oenococcus phage | 2036701, 1432848, 1432847, 2201414, 1435411, 1885654, 1885653, 1885652, |
| | 1885651, 1885650, 1885649, 1885648, 1885647, 1885646, 1885645, 1885644, |
| | 1885643, 1885642, 1885641, 1885640, 1303346 |
| Geobacillus phage | 1572712, 1965361, 365048, 1458842, 2686286 |
| Mycoplasma phage | 35238, 75590, 280702 |
| Klosneuvirus | 1977639 |
| Fowl aviadenovirus | 586029, 10553, 10547, 172857, 172859, 172860, 130663, 172861, 172862, |
| | 172863, 66295, 172864, 190061, 190062, 190063, 190064, 190065, 1972696 |

TABLE 14
Relative abundance of vial species listed in Table 16 and age of obese (Ob) subjects and
lean control (lean)

| | Achromobacter | Oenococcus | | Mycoplasma | | Fowl |
|---|---|---|---|---|---|---|
| group | phage | phage | Geobacillus phage | phage | Klosneuvirus | aviadenovirus |
| Lean | 2.82E−05 | 0.007996 | 0.011501 | 0 | 0.013441 | 2.97E−05 |
| Lean | 2.16E−06 | 0.022371 | 9.57E−04 | 0 | 0.004452 | 1.16E−04 |
| Lean | 1.73E−06 | 0.010301 | 0.003416 | 0 | 0.021655 | 1.43E−05 |
| Lean | 4.44E−06 | 0.013305 | 0.002099 | 6.39E−06 | 0.024338 | 1.37E−05 |
| Lean | 0 | 0.009906 | 0.004651 | 0 | 0.018631 | 3.43E−05 |
| Lean | 4.41E−06 | 0.008578 | 0.001461 | 6.43E−05 | 0.021187 | 2.21E−05 |
| Lean | 3.82E−06 | 0.00931 | 0.001693 | 4.30E−05 | 0.023346 | 9.18E−06 |
| Lean | 3.64E−05 | 0.011814 | 0.0023 | 5.98E−05 | 0.023836 | 1.82E−06 |
| ObT2 | 2.17E−05 | 0.00995 | 0.0051 | 3.54E−05 | 0.028791 | 1.79E−05 |
| ObT2 | 6.51E−06 | 0.0085 | 0.00266 | 6.98E−05 | 0.025446 | 1.19E−05 |
| ObT2 | 1.94E−04 | 0.011135 | 6.13E−04 | 1.39E−04 | 0.006819 | 1.90E−05 |
| ObT2 | 0 | 0.015196 | 0.008531 | 0 | 0.022655 | 5.37E−06 |
| Lean | 2.30E−04 | 0.01016 | 0.002861 | 4.27E−05 | 0.021006 | 5.27E−05 |
| Lean | 2.40E−05 | 0.007695 | 0.001981 | 2.14E−05 | 0.021183 | 4.90E−05 |
| ObT2 | 2.10E−04 | 0.013868 | 0.005288 | 4.38E−06 | 0.032934 | 3.14E−05 |
| ObT2 | 4.09 E−05 | 0.002428 | 0.001585 | 0 | 0.009372 | 3.21E−06 |
| ObT2 | 1.43E−05 | 0.002985 | 0.001078 | 3.75E−05 | 0.014465 | 1.79E−05 |
| ObT2 | 0 | 0.033672 | 0.001937 | 0 | 0.013946 | 5.04E−05 |
| ObT2 | 9.22E−05 | 0.00299 | 0.013036 | 3.93E−05 | 0.008352 | 3.89E−05 |
| ObT2 | 2.69E−05 | 0.00871 | 0.001207 | 0 | 0.020019 | 9.89E−06 |
| ObT2 | 5.52E−05 | 0.005935 | 8.63E−04 | 5.94E−04 | 0.016195 | 1.14E−04 |
| ObT2 | 1.73E−05 | 0.001472 | 7.66E−04 | 1.03E−05 | 0.025688 | 2.52E−05 |
| ObT2 | 0 | 0.006542 | 0.002988 | 3.13E−06 | 0.019793 | 1.80E−05 |
| ObT2 | 0 | 0.005078 | 0.005799 | 0 | 0.013167 | 2.27E−05 |
| ObT2 | 1.53E−05 | 0.012257 | 7.22E−04 | 1.13E−04 | 0.020212 | 5.27E−06 |
| ObT2 | 4.15E−06 | 0.01195 | 0.001079 | 3.16E−05 | 0.007767 | 6.60E−05 |
| ObT2 | 1.77E−05 | 0.007965 | 0.003167 | 0 | 0.022452 | 1.20E−04 |
| ObT2 | 1.24E−06 | 0.003527 | 0.005635 | 0 | 0.013235 | 1.03E−05 |
| ObT2 | 0 | 0.002704 | 9.74E−04 | 4.41E−04 | 0.00756 | 6.80E−05 |
| ObT2 | 1.91E−06 | 0.006455 | 7.48E−04 | 0 | 0.001125 | 8.29E−05 |
| ObT2 | 0 | 0.007553 | 8.69E−04 | 0 | 0.012343 | 6.21E−05 |
| ObT2 | 8.94E−06 | 0.009917 | 3.93E−04 | 0 | 0.006584 | 3.07E−06 |
| ObT2 | 8.83E−05 | 0.002343 | 3.11E−04 | 5.47E−06 | 0.031682 | 2.47E−05 |
| ObT2 | 8.48E−05 | 0.008693 | 0.001723 | 0 | 0.016627 | 6.30E−05 |
| ObT2 | 2.15E−06 | 0.002624 | 0.001755 | 0 | 0.006873 | 3.99E−05 |
| ObT2 | 6.04E−05 | 0.004935 | 0.003547 | 0 | 0.013586 | 1.15E−04 |
| ObT2 | 4.51E−04 | 0.005344 | 0.002787 | 0 | 0.01818 | 5.25E−05 |
| ObT2 | 5.71E−06 | 0.004648 | 0.002457 | 1.65E−05 | 0.011506 | 8.21E−05 |
| ObT2 | 2.23E−05 | 0.006576 | 0.007299 | 7.88E−05 | 0.013239 | 1.41E−05 |
| Lean | 0 | 0.024663 | 0.002334 | 0 | 0.009182 | 0 |
| Lean | 0 | 0.013313 | 0.006468 | 0 | 0.003988 | 1.25E−05 |
| Lean | 1.95E−05 | 0.006726 | 0.004954 | 0 | 0.006472 | 0 |
| Lean | 2.60E−05 | 0.00986 | 0.002666 | 6.02E−06 | 0.016682 | 5.75E−06 |
| Lean | 2.25E−06 | 0.007626 | 0.002271 | 0 | 0.028084 | 0 |
| Lean | 0 | 4.85 E−04 | 1.85E−04 | 0 | 0.003152 | 0 |
| Lean | 1.03E−05 | 0.008283 | 0.001588 | 0 | 0.009517 | 0 |
| Lean | 0 | 0.011772 | 0.001795 | 0 | 0.014987 | 1.63E−05 |
| Lean | 0 | 0.021353 | 0.007178 | 0 | 0.019018 | 0 |
| Lean | 4.78E−06 | 0.020756 | 0.002137 | 0 | 0.021183 | 0 |
| Lean | 1.64E−06 | 0.008847 | 0.001326 | 0.0022 | 0.02188 | 0 |
| Lean | 3.68E−06 | 0.002388 | 0.013958 | 1.66E−04 | 0.010477 | 0 |
| Lean | 1.95E−06 | 0.009731 | 0.002611 | 0 | 0.025696 | 1.00E−05 |
| Lean | 0 | 0.002078 | 0.006904 | 0 | 0.004543 | 0 |
| Lean | 3.54E−05 | 0.004664 | 0.002229 | 0 | 0.01565 | 1.38E−05 |
| Lean | 1.37E−05 | 2.36E−05 | 0.042081 | 0 | 0.001686 | 4.24E−05 |
| Lean | 1.33E−06 | 0.010106 | 0.002847 | 0 | 0.012446 | 8.54E−06 |
| Lean | 5.50E−04 | 0.005669 | 0 | 0 | 0.001366 | 0 |
| Lean | 0 | 0.003123 | 0.001589 | 0 | 0.011134 | 0 |
| Lean | 2.22E−06 | 0.008062 | 0.01774 | 0 | 0.008868 | 1.26E−05 |
| Lean | 0 | 3.78E−04 | 0.001337 | 0 | 0.001132 | 1.25E−05 |
| Lean | 3.75E−06 | 0.00348 | 0.001864 | 1.63E−04 | 0.010392 | 1.74E−05 |
| Lean | 9.02E−05 | 0.006732 | 0.002617 | 0 | 0.019311 | 1.07E−05 |
| Lean | 5.45E−07 | 0.022821 | 0.007078 | 0 | 0.031415 | 0 |
| Lean | 0 | 0.009465 | 7.04E−04 | 0 | 0.007843 | 0 |
| Lean | 6.23E−06 | 0.008264 | 0.001351 | 0 | 0.012241 | 8.57E−06 |
| Lean | 2.05E−06 | 0.003674 | 0.002921 | 2.95E−06 | 0.021078 | 1.91E−05 |
| Lean | 3.98E−05 | 0.010166 | 0.002815 | 2.52E−06 | 0.024633 | 7.24E−06 |
| Lean | 7.61E−07 | 0.009745 | 0.00364 | 4.38E−06 | 0.026931 | 0 |
| Lean | 0 | 0.006448 | 0.002672 | 2.77E−05 | 0.00965 | 1.98E−05 |
| Lean | 3.26E−05 | 0.00268 | 0.007466 | 2.21E−05 | 0.020536 | 9.04E−06 |
| Lean | 1.25E−05 | 0.019472 | 2.50E−04 | 0 | 0.002921 | 5.62E−05 |
| Lean | 6.03E−06 | 0.00329 | 0.002347 | 0 | 0.014638 | 7.95E−05 |
| Lean | 8.10E−06 | 0.012082 | 0.002407 | 2.56E−06 | 0.027198 | 0 |
| Lean | 5.61E−06 | 0.009469 | 0.002409 | 4.84E−06 | 0.018906 | 0 |
| Lean | 4.19E−06 | 0.004623 | 0.001326 | 0 | 0.043265 | 0 |
| Lean | 4.72E−06 | 0.005786 | 0.001926 | 0 | 0.01638 | 1.95E−05 |
| Lean | 0 | 0.007083 | 0.003611 | 0 | 0.022715 | 3.33E−05 |
| Lean | 2.73E−05 | 0.008374 | 0.001541 | 3.19E−06 | 0.006356 | 2.29E−05 |
| Lean | 0 | 0.004317 | 0.001331 | 0 | 0.018038 | 3.69E−05 |
| Lean | 0 | 0.006532 | 0.002899 | 0 | 0.015261 | 1.90E−06 |
| Lean | 1.75E−05 | 0.014799 | 0.005749 | 5.39E−05 | 0.038981 | 0 |
| Lean | 7.59E−06 | 0.005572 | 0.002846 | 2.05E−05 | 0.021693 | 4.50E−05 |
| Lean | 3.51E−05 | 0.005512 | 0.002318 | 0 | 0.018606 | 7.52E−05 |
| Lean | 0 | 0.009558 | 0.003608 | 0 | 0.020998 | 0 |
| Lean | 1.85E−06 | 0.014457 | 0.003579 | 0 | 0.024622 | 1.53E−05 |
| Lean | 1.19E−06 | 0.016044 | 0.008419 | 0 | 0.01993 | 0 |
| Lean | 0 | 0.018982 | 0.006574 | 0 | 0.012631 | 0 |
| Lean | 2.82E−06 | 0.004169 | 0.00161 | 0 | 0.015117 | 1.94E−06 |
| Lean | 4.75E−06 | 0.006396 | 0.008516 | 2.76E−04 | 0.013254 | 0 |
| Lean | 8.04E−06 | 0.007436 | 0.00133 | 0 | 0.006818 | 7.46E−05 |
| Lean | 3.29E−05 | 0.006285 | 0.002284 | 0 | 0.017173 | 1.43E−04 |
| Lean | 0 | 0.010328 | 0.005378 | 0 | 0.026672 | 2.63E−05 |
| Lean | 3.95E−05 | 0.002773 | 0.001097 | 1.39E−05 | 0.022887 | 7.06E−05 |
| Lean | 2.42E−05 | 0.007892 | 0.004259 | 1.23E−05 | 0.026422 | 5.87E−06 |
| Lean | 1.21E−04 | 0.004829 | 0.004137 | 0 | 0.019326 | 1.31E−04 |
| ObT2 | 0 | 0.002601 | 5.84E−04 | 1.44E−06 | 3.15E−04 | 0 |
| ObT2 | 1.93E−04 | 0.008448 | 0.002392 | 2.09E−05 | 0.010696 | 1.58E−04 |
| ObT2 | 3.92E−05 | 0.004357 | 0.001919 | 0 | 0.010513 | 5.39E−06 |
| ObT2 | 2.49E−06 | 0.011753 | 0.003621 | 1.17E−04 | 0.030835 | 7.37E−05 |
| ObT2 | 4.63E−05 | 0.002441 | 0.001475 | 2.38E−06 | 0.011707 | 3.75E−05 |
| ObT2 | 0 | 0.013913 | 0.02458 | 0.002114 | 0.024082 | 1.22E−04 |
| ObT2 | 5.10E−04 | 0.00163 | 6.18E−04 | 0 | 0.004425 | 7.03E−05 |
| ObT2 | 0.007325 | 4.57E−04 | 0 | 0 | 0.00173 | 0 |
| ObT2 | 4.23E−05 | 3.15E−04 | 8.17E−05 | 5.09E−04 | 0.004157 | 3.43E−05 |
| ObT2 | 0 | 9.31E−04 | 4.96E−04 | 0.02639 | 0.001283 | 0 |
| ObT2 | 1.29E−05 | 0.001745 | 7.78E−04 | 0 | 0.001099 | 0 |
| ObT2 | 4.84E−06 | 0.009492 | 0.003715 | 9.86E−05 | 0.022782 | 0 |
| ObT2 | 1.70E−04 | 6.00E−04 | 4.42 E−04 | 0 | 0.003983 | 1.06E−04 |
| ObT2 | 1.03E−04 | 0.009829 | 0.001996 | 0 | 0.021409 | 1.77E−04 |
| ObT2 | 1.55E−05 | 0.008122 | 0.001538 | 0 | 0.008074 | 1.75E−04 |
| ObT2 | 0 | 0.004329 | 0.001584 | 2.95E−05 | 0.018093 | 8.30E−05 |
| ObT2 | 5.85E−05 | 0.002629 | 0.001172 | 0.001263 | 0.04466 | 6.41E−05 |
| ObT2 | 2.34E−04 | 0.003172 | 0.002855 | 0 | 0.006852 | 2.94E−04 |
| ObT2 | 1.18E−06 | 0.002964 | 6.38E−04 | 8.48E−06 | 0.014392 | 2.43E−06 |
| ObT2 | 5.24E−06 | 0.001456 | 7.43E−04 | 2.49E−05 | 0.026748 | 1.73E−05 |
| ObT2 | 2.61E−04 | 0.010825 | 0.002243 | 0 | 0.036694 | 0 |
| ObT2 | 2.05E−05 | 0.004272 | 9.30E−04 | 6.93E−05 | 0.01091 | 6.81E−05 |
| ObT2 | 2.63E−04 | 0.010132 | 0.003806 | 1.94E−05 | 0.01369 | 1.30E−04 |
| ObT2 | 2.34E−04 | 0.001701 | 3.87E−04 | 0 | 0.006793 | 1.33E−04 |
| ObT2 | 2.64E−05 | 0.003118 | 3.58E−04 | 0 | 0.001351 | 1.95E−05 |
| ObT2 | 2.05E−06 | 0.050797 | 4.23E−05 | 0 | 2.32E−04 | 1.31E−05 |
| ObT2 | 1.87E−05 | 0.001446 | 0.002627 | 2.77E−04 | 0.008339 | 2.59E−04 |
| ObT2 | 0 | 0.0054 | 9.35E−04 | 0 | 0.009478 | 2.73E−05 |
| ObT2 | 1.90E−05 | 0.003429 | 0.00339 | 1.42E−04 | 0.011077 | 8.94E−07 |
| ObT2 | 1.61E−04 | 0.01057 | 0.001892 | 2.02E−04 | 0.020124 | 4.66E−05 |
| ObT2 | 2.78E−05 | 0.010387 | 0.002856 | 1.06E−04 | 0.017218 | 2.95E−05 |
| ObT2 | 9.16E−05 | 0.001338 | 6.99 E−04 | 0 | 0.001881 | 5.06E−06 |
| ObT2 | 0 | 0.012655 | 0.002464 | 6.48E−06 | 0.017464 | 4.65E−05 |
| ObT2 | 1.34E−06 | 0.00191 | 4.84E−04 | 5.30E−05 | 0.003987 | 3.18E−05 |
| ObT2 | 1.09E−05 | 0.004102 | 9.32E−04 | 0 | 0.001855 | 6.29E−05 |
| ObT2 | 1.05E−05 | 0.006962 | 3.67E−04 | 0 | 0.00323 | 1.09E−04 |
| ObT2 | 1.92E−06 | 0.001534 | 9.18E−04 | 3.54E−04 | 0.036841 | 8.89 E−06 |
| ObT2 | 1.11E−04 | 0.005911 | 0.002988 | 2.21E−04 | 0.013483 | 2.95E−05 |
| ObT2 | 2.00E−05 | 0.009536 | 0.001812 | 0 | 0.014113 | 1.48E−04 |
| ObT2 | 3.18E−05 | 0.003201 | 0.003291 | 4.57E−05 | 0.008068 | 2.66E−05 |
| ObT2 | 9.06E−06 | 0.002159 | 0.001241 | 0.001654 | 0.007256 | 2.17E−04 |
| ObT2 | 5.99E−06 | 0.009897 | 0.003244 | 6.26E−06 | 0.028931 | 1.12E−05 |
| ObT2 | 3.14E−05 | 0.002399 | 0.006387 | 9.78E−06 | 0.01113 | 6.31E−05 |
| ObT2 | 8.80E−05 | 0.00249 | 0.007052 | 5.20E−05 | 0.006962 | 0 |
| ObT2 | 1.68E−04 | 0.00259 | 0.001567 | 1.11E−05 | 0.00789 | 0 |
| Lean | 6.43E−07 | 0.009499 | 0.001409 | 0 | 0.004294 | 0 |
| Lean | 1.35E−06 | 7.68E−04 | 0.001517 | 0 | 0.005584 | 0 |
| Lean | 1.05E−05 | 0.003745 | 0.001889 | 0 | 0.014798 | 1.38E−05 |
| Lean | 9.94E−07 | 0.006407 | 0.003379 | 0 | 0.016714 | 0 |
| Lean | 1.23E−05 | 0.010489 | 0.002576 | 1.96E−04 | 0.016947 | 4.23E−06 |
| Lean | 2.36E−06 | 0.007293 | 0.002684 | 0 | 0.0157 | 2.19E−05 |
| Lean | 0 | 0.008382 | 0.002627 | 0 | 0.005408 | 8.02E−06 |
| Lean | 1.18E−05 | 0.004121 | 0.001937 | 0 | 0.008856 | 2.03E−06 |
| Lean | 0 | 0.024416 | 0.00118 | 0 | 0.003989 | 6.73E−06 |
| Lean | 7.21E−06 | 0.009733 | 0.002751 | 2.07E−06 | 0.015203 | 2.97E−06 |
| Lean | 2.96E−06 | 0.01926 | 0.003049 | 0 | 0.010665 | 0 |
| Lean | 0 | 0.023932 | 0.006561 | 0 | 0.015793 | 0 |
| Lean | 6.80E−07 | 0.006392 | 0.002006 | 9.78E−07 | 0.035148 | 4.21E−06 |
| Lean | 7.29E−07 | 0.005937 | 0.001867 | 5.55E−04 | 0.014468 | 9.03E−06 |
| Lean | 0 | 0.006481 | 0.001375 | 0 | 0.009125 | 0 |
| Lean | 2.15E−06 | 1.20E−04 | 2.44E−05 | 0 | 7.15E−04 | 3.44E−05 |
| Lean | 0 | 0 | 2.86E−04 | 0 | 0.009215 | 0 |
| Lean | 2.84E−06 | 0.005091 | 0.001435 | 0 | 0.014185 | 3.52E−06 |
| Lean | 3.06E−06 | 0.003203 | 0.003758 | 5.50E−06 | 0.008431 | 0 |
| Lean | 2.13E−05 | 0.003732 | 0.003925 | 0 | 0.014439 | 1.70E−05 |
| Lean | 0 | 0 | 0 | 0 | 0.058426 | 0 |
| Lean | 0 | 0.00283 | 0.005065 | 0 | 0.007066 | 0 |
| Lean | 5.37E−05 | 0.005988 | 0.003671 | 0 | 0.017244 | 0 |
| Lean | 5.25E−05 | 0.003822 | 0.002373 | 0 | 0.010978 | 2.38E−05 |
| Lean | 1.90E−04 | 0.014019 | 0.001598 | 0 | 0.008752 | 1.07E−06 |
| Lean | 1.13E−05 | 0.013581 | 0.002496 | 0 | 0.026521 | 2.38E−05 |
| Lean | 2.03E−06 | 0.002577 | 5.05E−04 | 0 | 0.014075 | 0 |
| Lean | 0 | 0.015924 | 0.005717 | 0 | 0.027881 | 1.02E−05 |
| Lean | 3.41E−05 | 0.002899 | 0.005702 | 0 | 0.011395 | 1.62E−05 |
| Lean | 3.81E−06 | 0.016288 | 0.002617 | 0 | 0.019974 | 3.71E−04 |
| Lean | 0 | 0.01031 | 0.001867 | 2.37E−05 | 0.014556 | 1.51E−05 |
| Lean | 4.77E−05 | 0.007257 | 0.00168 | 7.69E−05 | 0.015301 | 9.24E−05 |
| Lean | 0 | 0.007514 | 0.002947 | 0 | 0.016136 | 6.01E−05 |
| Lean | 3.16E−05 | 0.005387 | 0.002588 | 2.43E−04 | 0.020113 | 3.62E−06 |
| Lean | 0 | 0.01221 | 0.001344 | 0 | 0.016138 | 4.05E−05 |

As such, to determine the risk of obesity with type 2 diabetes (ObT2) in a subject, the following steps will be carried out:
1. Obtain a set of training data by determine the relative abundance of species selected from Table 13* in a cohort of obese with type 2 diabetes (ObT2) subjects and lean controls.
2. Determine the relative abundance of these species in the subject whose risk of obesity is to be determined.
3. Compare the relative abundance of these species in the subject with the training data using random forest model.
4. Decision trees will be generated by random forest from the training data. The relative abundances will be run down the decision trees and generate a risk score. If more than 50% trees in the model consider the subject obese with type 2 diabetes, the outcome will be “subject being tested is deemed to be at an increased risk for obesity with type 2 diabetes”. If less than 50% trees in the model consider the subject as lean, the outcome will be “subject being tested is deemed to be at low risk for obesity with type 2 diabetes”.
   *species selected from Table 13 comprise of
   1. Achromobacter phage, Oenococcus phage, Geobacillus phage (top 3 markers; AUC: 90.41%; FIG. 23);
   2. Achromobacter phage, Oenococcus phage, Geobacillus phage, Mycoplasma phage (top 4 markers; AUC: 91.45%; FIG. 23);
   3. Achromobacter phage, Oenococcus phage, Geobacillus phage, Mycoplasma phage, Klosneuvirus (top 5 markers; AUC: 91.87%; FIG. 23); or
   4. Achromobacter phage, Oenococcus phage, Geobacillus phage, Mycoplasma phage, Klosneuvirus, Fowl aviadenovirus (all 6 markers; AUC: 93.2%; FIG. 23).

### EXAMPLE

The relative abundance of 6 species listed in Table 13 from Lean (n=101) and ObT2 (n=74) was determined by metagenomics sequencing and taxonomy assigned as described in methods (relative abundance listed in Table 14). Decision trees were generated by random forest from data in Table 13 with parameter: trees=1000, mtry=4.

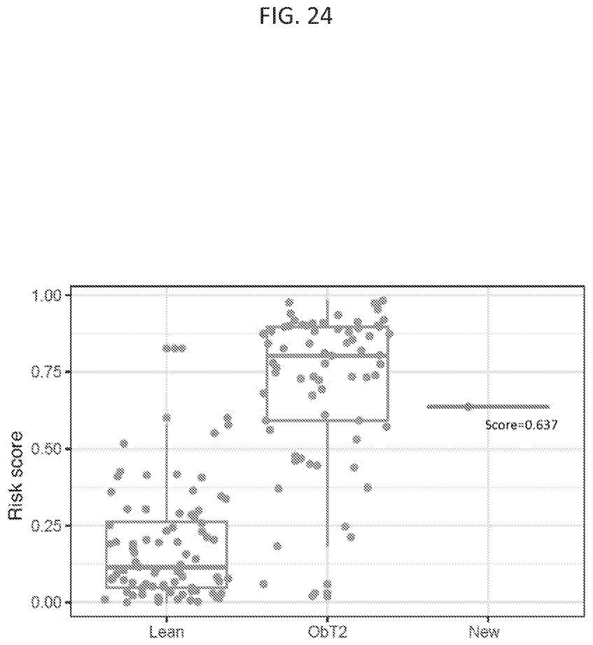

The likelihood of having obesity with type 2 diabetes in a 57-year-old male a subject (FB006) was determined. The relative abundance of the 5 species listed in Table 13 in fecal sample of this subject was determined by metagenomics sequencing and taxonomy assigned as described in method. Relative abundance of the 6 species in this subject is shown in Table 15. The relative abundances were run down the decision trees and a risk score was generated using relative abundance in Table 14 as training data. The score of the subject was 0.637 (FIG. 24), and therefore the subject was deemed to have a medium risk for obesity combined with T2DM. The subject has a BMI of 35.6 and was diagnosed with type 2 diabetes.

TABLE 15
Relative abundance of 6 vial species listed in Table 16 of a subject whose risk
of obesity with type 2 diabetes is to be determined

| Achromobacter | Oenococcus | Geobacillus | Mycoplasma | | Fowl |
|---|---|---|---|---|---|
| phage | phage | phage | phage | Klosneuvirus | aviadenovirus |
| 8.26E−06 | 0.004647273 | 0.001886758 | 1.76E−04 | 0.009639865 | 3.07E−05 |

Model 3: Obese with Type 2 Diabetes (ObT2) Vs Obese (Ob)

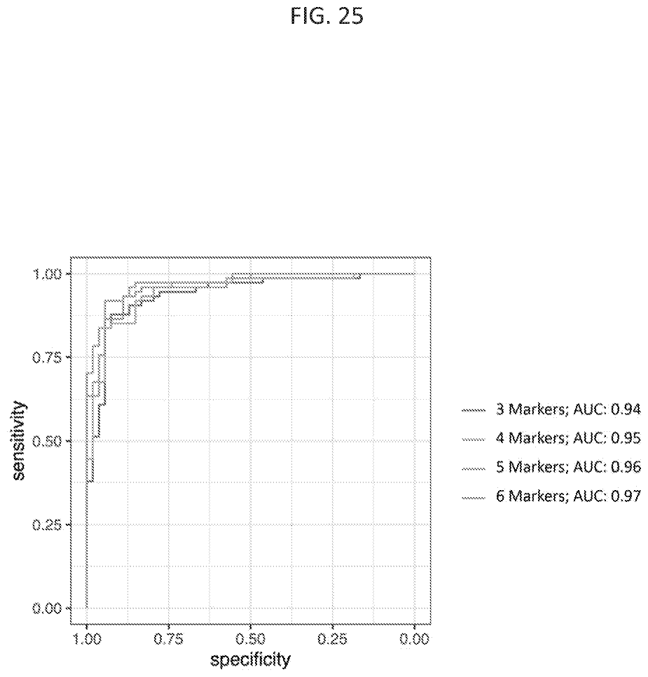

A total of 74 ObT2 and 54 Ob subjects were included as the discovery cohort for modelling. Five viral markers, including Oenococcus phage, Bradyrhizobium phage, Phormidium phage, Heliothis zea nudivirus and Achromobacter phage, and age were included in the machine learning model (Table 16). The final models using these 6 markers has an Area Under the Curve (AUC) in Receiver Operating Characteristic (ROC) curves of 97.22% (FIG. 25).

TABLE 16
Viral species included in the machine learning model for prediction of type 2
diabetes in obese subjects

| Viral Species | NCBI:txid |
|---|---|
| Oenococcus phage | 2036701, 1432848, 1432847, 2201414, 1435411, 1885654, 1885653, |
| | 1885652, 1885651, 1885650, 1885649, 1885648, 1885647, 1885646, |
| | 1885645, 1885644, 1885643, 1885642, 1885641, 1885640, 1303346 |
| Bradyrhizobium phage | 1983459 |
| Phormidium phage | 400567, 440250, 394231, 1391456, 1391455 |
| Heliothis zea nudivirus | 29250 |
| Achromobacter phage | 1589747, 2723726, 2723727, 2292880, 1664247, 1664246, 1610509, 2591403, |
| | 1589746, 1589748, 1416008, 1416009, 2723728, 2591054, 2591053, 2591039, |
| | 2591040, 2591041, 2591042, 2591043, 2591044, 2591045, 2591046, 2591047, |
| | 2591048, 2591049, 2591050, 2591051, 2591052 |

TABLE 17
Relative abundance of vial species listed in Table 19 and age of subjects with obese with type 2
diabetes (ObT2) and obesity alone (Ob)

| | | Oenococcus | Bradyrhizobium | Phormidium | Heliothis zea | Achromobacter |
|---|---|---|---|---|---|---|
| group | age | phage | phage | phage | nudivirus | phage |
| Ob | 57 | 0.011676 | 0 | 0.000101 | 8.42E−06 | 4.81E−05 |
| ObT2 | 57 | 0.0085 | 6.25E−06 | 0 | 0 | 6.51E−06 |
| Ob | 29 | 0.009617 | 0.000037 | 0 | 0.000202 | 1.17E−05 |
| ObT2 | 30 | 0.013913 | 0 | 0 | 0.000208 | 0 |
| Ob | 37 | 0.001961 | 0 | 0 | 0.000247 | 3.16E−05 |
| Ob | 25 | 0.004647 | 0 | 0 | 3.84E−05 | 0 |
| Ob | 29 | 0.00733 | 0 | 0 | 0.000115 | 0 |
| Ob | 47 | 0.005402 | 0 | 0 | 3.48E−06 | 0 |
| Ob | 29 | 0.004974 | 0.000195 | 0.005181 | 0.000271 | 1.45E−05 |
| Ob | 27 | 0.009416 | 0 | 0 | 1.72E−05 | 1.48E−06 |
| Ob | 20 | 0.00375 | 0 | 0.000313 | 1.35E−05 | 0 |
| Ob | 30 | 0.007728 | 0 | 0 | 0 | 0 |
| Ob | 50 | 0.011491 | 0 | 0 | 0 | 0 |
| Ob | 27 | 0.009689 | 0 | 0.002041 | 5.33E−05 | 0 |
| Ob | 42 | 0.013275 | 0.000209 | 0 | 4.99E−06 | 7.98E−07 |
| ObT2 | 58 | 0.013868 | 0 | 0.000262 | 2.29E−05 | 0.00021 |
| Ob | 32 | 0.004373 | 8.53E−05 | 1.57E−05 | 0.00027 | 0.000035 |
| Ob | 51 | 0.010529 | 0 | 0.001488 | 7.17E−06 | 6.36E−07 |
| Ob | 51 | 0.011181 | 0 | 0 | 0 | 0 |
| ObT2 | 55 | 0.002428 | 0 | 0.000026 | 0 | 4.09E−05 |
| Ob | 39 | 0.006111 | 0 | 6.32E−05 | 0 | 4.46E−06 |
| Ob | 61 | 0.002612 | 0 | 0.000152 | 0 | 7.79E−06 |
| Ob | 32 | 0.010735 | 2.18E−06 | 0.000656 | 7.65E−06 | 6.56E−07 |
| Ob | 29 | 0.007736 | 0 | 0 | 0.000657 | 0 |
| Ob | 66 | 0.019965 | 1.67E−05 | 0 | 9.75E−07 | 0 |
| ObT2 | 57 | 0.009492 | 2.53E−06 | 6.56E−06 | 0.00024 | 4.84E−06 |
| ObT2 | 50 | 0.009829 | 0.000053 | 0 | 0 | 0.000103 |
| ObT2 | 51 | 0.00299 | 3.28E−05 | 0 | 0 | 9.22E−05 |
| ObT2 | 36 | 0.002629 | 1.85E−06 | 0.000108 | 0.000362 | 5.85E−05 |
| ObT2 | 41 | 0.003172 | 0.000331 | 2.11E−05 | 0.000422 | 0.000234 |
| ObT2 | 63 | 0.00871 | 1.66E−05 | 0.000144 | 0.000459 | 2.69E−05 |
| ObT2 | 52 | 0.002964 | 1.64E−05 | 0 | 0.000134 | 1.18E−06 |
| ObT2 | 59 | 0.004272 | 5.06E−05 | 0 | 8.94E−06 | 2.05E−05 |
| ObT2 | 64 | 0.006542 | 6.58E−05 | 0.00056 | 3.63E−06 | 0 |
| ObT2 | 61 | 0.005078 | 0 | 0 | 0.00086 | 0 |
| ObT2 | 40 | 0.003118 | 0.000242 | 0 | 0.003151 | 2.64E−05 |
| ObT2 | 53 | 0.050797 | 0 | 0 | 0.000131 | 2.05E−06 |
| ObT2 | 49 | 0.010387 | 3.35E−05 | 0 | 0.000963 | 2.78E−05 |
| ObT2 | 60 | 0.006455 | 7.59E−05 | 7.97E−05 | 0 | 1.91E−06 |
| ObT2 | 67 | 0.001338 | 0.000021 | 0 | 0.000727 | 9.16E−05 |
| ObT2 | 55 | 0.012655 | 0 | 0 | 0.001735 | 0 |
| ObT2 | 54 | 0.007553 | 4.13E−06 | 0 | 0.000017 | 0 |
| ObT2 | 70 | 0.006962 | 3.04E−05 | 0 | 0 | 1.05E−05 |
| ObT2 | 50 | 0.004935 | 4.31E−05 | 8.38E−05 | 0.000696 | 6.04E−05 |
| ObT2 | 64 | 0.005344 | 0.000242 | 0.001115 | 0.005155 | 0.000451 |
| ObT2 | 33 | 0.005911 | 0 | 0 | 0.001038 | 0.000111 |
| ObT2 | 41 | 0.009536 | 0 | 0 | 0.000174 | 0.00002 |
| ObT2 | 46 | 0.002159 | 0.000191 | 0 | 0 | 9.06E−06 |
| ObT2 | 60 | 0.002399 | 4.72E−05 | 0.00036 | 0.000727 | 3.14E−05 |
| ObT2 | 45 | 0.006576 | 4.85E−05 | 0 | 0.000328 | 2.23E−05 |
| Ob | 45 | 0.005808 | 1.06E−05 | 0.000106 | 0.000444 | 8.33E−05 |
| Ob | 53 | 0.006666 | 0 | 0.000431 | 0 | 0 |
| ObT2 | 44 | 0.00995 | 1.95E−05 | 0.000537 | 0.000584 | 2.17E−05 |
| ObT2 | 37 | 0.002601 | 0 | 0 | 0 | 0 |
| ObT2 | 49 | 0.008448 | 0 | 0 | 0.000367 | 0.000193 |
| Ob | 63 | 0.022831 | 0 | 0 | 0 | 0 |
| ObT2 | 59 | 0.004357 | 0.000057 | 0.00031 | 5.89E−05 | 3.92E−05 |
| ObT2 | 32 | 0.011135 | 0.001526 | 0 | 0.000831 | 0.000194 |
| ObT2 | 62 | 0.011753 | 0 | 0 | 0.000753 | 2.49E−06 |
| Ob | 34 | 0.007446 | 4.38E−05 | 0.000544 | 0 | 2.59E−06 |
| ObT2 | 60 | 0.002441 | 9.07E−05 | 0 | 0 | 4.63E−05 |
| Ob | 32 | 0.006993 | 1.22E−05 | 0 | 0.001334 | 1.71E−05 |
| Ob | 27 | 0.006782 | 0.000117 | 0.000662 | 0 | 1.29E−05 |
| ObT2 | 43 | 0.015196 | 0 | 0 | 0.002578 | 0 |
| Ob | 50 | 0.019179 | 1.88E−05 | 9.83E−05 | 1.29E−05 | 9.87E−06 |
| ObT2 | 50 | 0.00163 | 0.001175 | 1.44E−05 | 0.000224 | 0.00051 |
| Ob | 52 | 0.01459 | 0 | 0 | 4.85E−05 | 1.63E−06 |
| Ob | 23 | 0.005136 | 0 | 0.000633 | 1.97E−06 | 1.57E−06 |
| Ob | 22 | 0.016336 | 0 | 0 | 3.09E−05 | 0 |
| Ob | 29 | 0.003235 | 0 | 0.001695 | 2.07E−05 | 0.000063 |
| Ob | 53 | 0.000103 | 0.000695 | 0 | 0.000185 | 0 |
| Ob | 21 | 0.006395 | 0 | 0 | 2.16E−05 | 0 |
| Ob | 56 | 0.006474 | 0 | 0.002092 | 1.49E−05 | 2.71E−06 |
| Ob | 22 | 0.004053 | 0 | 0.000949 | 0 | 1.49E−06 |
| Ob | 27 | 0.008162 | 0 | 0 | 0 | 0 |
| Ob | 27 | 0.00334 | 7.48E−06 | 0.000817 | 6.5E−06 | 3.46E−06 |
| Ob | 33 | 0.007669 | 1.24E−05 | 0.001022 | 4.85E−05 | 1.08E−06 |
| Ob | 22 | 0.004393 | 0 | 0 | 0 | 0 |
| Ob | 31 | 0.004091 | 1.99E−06 | 0 | 0.000014 | 0 |
| Ob | 40 | 0.006472 | 0 | 1.75E−05 | 8.77E−06 | 1.08E−05 |
| Ob | 69 | 0.006748 | 4.48E−06 | 0 | 0 | 0 |
| ObT2 | 61 | 0.000457 | 0 | 0 | 0 | 0.007325 |
| Ob | 26 | 0.016244 | 8.63E−05 | 0.000886 | 6.18E−06 | 1.21E−05 |
| Ob | 37 | 0.003847 | 1.49E−05 | 0 | 0 | 1.46E−05 |
| ObT2 | 53 | 0.000315 | 0 | 0 | 0 | 4.23E−05 |
| Ob | 46 | 0.013324 | 0 | 0.000776 | 0 | 0 |
| Ob | 52 | 0.013304 | 1.49E−05 | 0 | 9.36E−05 | 0 |
| Ob | 28 | 0.00795 | 0 | 0.00003 | 0 | 0 |
| Ob | 41 | 0.009358 | 3.75E−06 | 0 | 0.00028 | 1.3E−06 |
| Ob | 52 | 0.008727 | 0 | 0 | 6.3E−06 | 0 |
| Ob | 37 | 0.006012 | 0 | 0.000166 | 4.08E−05 | 6.02E−05 |
| Ob | 55 | 0.009843 | 0 | 5.27E−05 | 2.74E−06 | 2.92E−06 |
| ObT2 | 37 | 0.000931 | 0 | 0 | 0.000406 | 0 |
| Ob | 33 | 0.006439 | 0 | 0 | 7.07E−06 | 0 |
| ObT2 | 30 | 0.001745 | 3.61E−05 | 0 | 3.59E−06 | 1.29E−05 |
| Ob | 61 | 0.004662 | 1.06E−05 | 0.000825 | 9.24E−06 | 0 |
| ObT2 | 66 | 0.002985 | 7.18E−05 | 0 | 0 | 1.43E−05 |
| ObT2 | 54 | 0.0006 | 0.000231 | 0 | 0 | 0.00017 |
| ObT2 | 56 | 0.033672 | 0 | 0 | 0 | 0 |
| ObT2 | 51 | 0.008122 | 0.000033 | 0 | 0.000763 | 1.55E−05 |
| ObT2 | 47 | 0.004329 | 7.44E−06 | 0 | 0.006045 | 0 |
| ObT2 | 69 | 0.005935 | 4.81E−05 | 0 | 0 | 5.52E−05 |
| ObT2 | 54 | 0.001456 | 0 | 8.43E−05 | 0 | 5.24E−06 |
| ObT2 | 47 | 0.001472 | 6.21E−06 | 0.000166 | 0 | 1.73E−05 |
| ObT2 | 34 | 0.010825 | 3.71E−05 | 0 | 3.43E−06 | 0.000261 |
| ObT2 | 39 | 0.010132 | 0.000422 | 0 | 0.001177 | 0.000263 |
| ObT2 | 61 | 0.001701 | 9.49E−05 | 0 | 0 | 0.000234 |
| ObT2 | 68 | 0.012257 | 1.23E−05 | 0.000246 | 3.2E−06 | 1.53E−05 |
| ObT2 | 57 | 0.001446 | 0 | 0 | 0.001739 | 1.87E−05 |
| ObT2 | 36 | 0.0054 | 0.000671 | 0 | 0.001679 | 0 |
| ObT2 | 45 | 0.01195 | 5.72E−05 | 9.88E−06 | 0 | 4.15E−06 |
| ObT2 | 44 | 0.007965 | 6.21E−06 | 3.29E−05 | 0.000317 | 1.77E−05 |
| ObT2 | 67 | 0.003429 | 0.00012 | 0 | 0.000223 | 0.000019 |
| ObT2 | 41 | 0.01057 | 0.00024 | 0 | 0 | 0.000161 |
| ObT2 | 65 | 0.003527 | 2.33E−05 | 0 | 0 | 1.24E−06 |
| ObT2 | 68 | 0.002704 | 0 | 0 | 0.002752 | 0 |
| ObT2 | 59 | 0.009917 | 1.72E−05 | 0 | 0 | 8.94E−06 |
| ObT2 | 43 | 0.002343 | 3.44E−05 | 0 | 0.000142 | 8.83E−05 |
| ObT2 | 54 | 0.00191 | 0.000285 | 0 | 0.000902 | 1.34E−06 |
| ObT2 | 62 | 0.008693 | 0.000147 | 8.83E−05 | 7.02E−06 | 8.48E−05 |
| ObT2 | 47 | 0.004102 | 3.64E−05 | 0 | 0.000422 | 1.09E−05 |
| ObT2 | 61 | 0.002624 | 4.35E−05 | 0 | 0.002805 | 2.15E−06 |
| ObT2 | 57 | 0.001534 | 8.28E−06 | 1.06E−05 | 6E−07 | 1.92E−06 |
| ObT2 | 58 | 0.003201 | 2.62E−05 | 0 | 0 | 3.18E−05 |
| ObT2 | 61 | 0.004648 | 1.74E−05 | 6.18E−05 | 0.000116 | 5.71E−06 |
| ObT2 | 53 | 0.009897 | 2.91E−05 | 9.06E−05 | 0.000309 | 5.99E−06 |
| ObT2 | 69 | 0.00249 | 7.74E−05 | 0 | 9.25E−05 | 0.000088 |
| ObT2 | 65 | 0.00259 | 0.000674 | 0 | 0.002474 | 0.000168 |

As such, to determine the risk of type 2 diabetes (ObT2) in a subject, the following steps will be carried out:
1. Confirm the subject to be tested has obesity or not. If the subject do not have obesity, should use model 1 or model 2. If the subject already shown sign of obesity, use this model.
2. Obtain a set of training data by determine the relative abundance of species selected from Table 16* in a cohort of obese with type 2 diabetes (ObT2) subjects and obese (Ob) subjects.
3. Determine the relative abundance of these species in the obese subject whose risk of type 2 diabetes is to be determined.
4. Compare the relative abundance of these species in the subject with the training data using random forest model.
5. Decision trees will be generated by random forest from the training data. The relative abundances will be run down the decision trees and generate a risk score. If more than 50% trees in the model consider the obese subject with type 2 diabetes, the outcome will be “subject being tested is deemed to be at an increased risk for type 2 diabetes”. If less than 50% trees in the model consider the subject as obese, the outcome will be “subject being tested is deemed to be at low risk for type 2 diabetes”.
   *species selected from Table 16 comprise of
   1. Age, Oenococcus phage, Bradyrhizobium phage (top 3 markers; AUC: 93.87%; FIG. 25);
   2. Age, Oenococcus phage, Bradyrhizobium phage, Phormidium phage (top 4 markers; AUC: 94.77%; FIG. 25);
   3. Age, Oenococcus phage, Bradyrhizobium phage, Phormidium phage, Heliothis zea nudivirus (top 5 markers; AUC: 96%; FIG. 25); or
   4. Age, Oenococcus phage, Bradyrhizobium phage, Phormidium phage, Heliothis zea nudivirus, Achromobacter phage (all 6 markers; AUC: 97.22%; FIG. 25)

### EXAMPLE

The relative abundance of 5 species listed in Table 16 from Ob (n=54) and ObT2 (n=74) was determined by metagenomics sequencing and taxonomy assigned as described in methods (relative abundance listed in Table 17). Decision trees were generated by random forest from data in Table 16 with parameter: trees=1000, mtry=4.

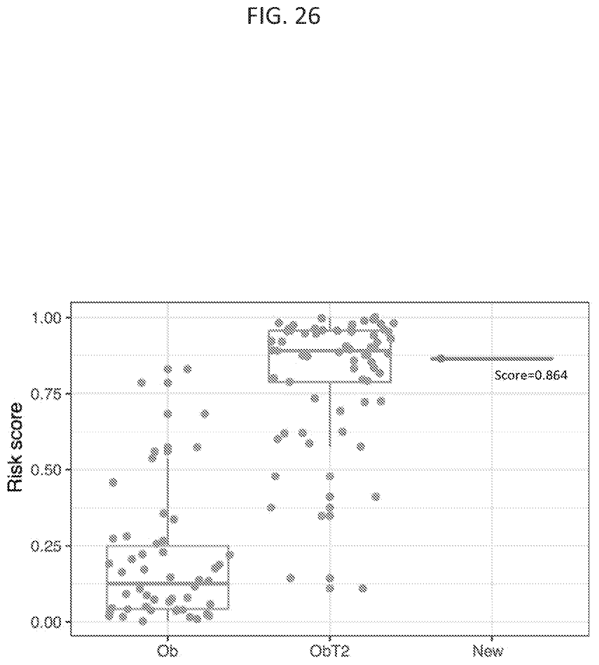

The likelihood of having obesity with T2DM in a 46-year-old male subject (FB001) was determined. The relative abundance of the 5 species listed in Table 16 in fecal sample of this subject was determined by metagenomics sequencing and taxonomy assigned as described in method. The relative abundances were run down the decision trees and a risk score was generated using relative abundance in Table 17 as training data. The score of the subject was 0.864 (FIG. 26), and therefore the subject was deemed to have a higher risk of obesity combined with T2DM. This subject had a BMI of 37 and was diagnosed with T2DM.

TABLE 18
Relative abundance of 5 vial species listed in Table 16 and age of a subject
whose risk of obesity with type 2 diabetes is to be determined

| | | | | Heliothis | |
|---|---|---|---|---|---|
| | Oenococcus | Bradyrhizobium | Phormidium | zea | Achromobacter |
| age | phage | phage | phage | nudivirus | phage |
| 46 | 0.006557 | 5.67E−05 | 1.16E−05 | 1.02E−04 | 2.15E−05 |

### Decreased Viral-Bacterial Inter-Kingdom Ecological Interactions in Obesity

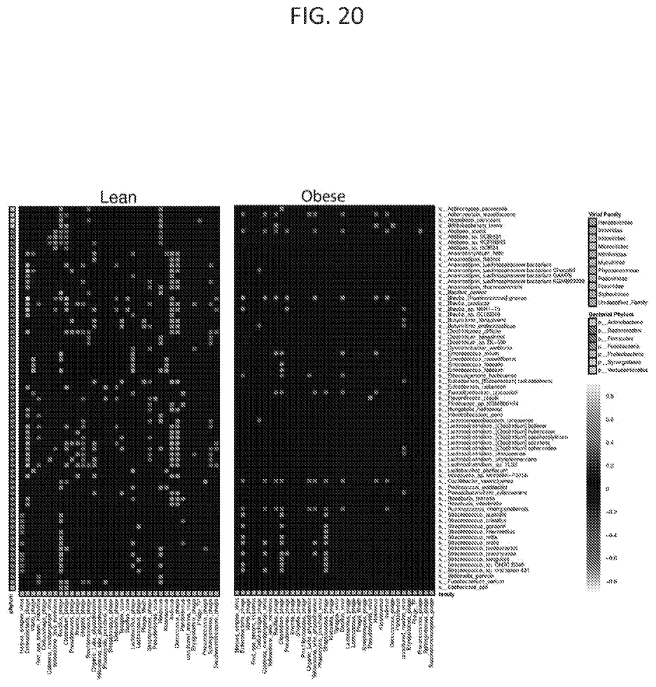

Studies have shown complex gut viral-bacterial ecological interactions beyond phage-host interactions in health and disease. The gut viral-bacterial inter-kingdom correlations in obese subjects and lean controls were calculated to explore associations between gut virome and bacteriome. It was discovered that decreased number of correlations between virome and bacteriome in obese subjects compared with lean controls (106 vs 317; Chi-squared test, p<0.001; FIG. 20). Among the significant inter-kingdom correlations, 49.1% (n=52) were positive (co-occurrence) correlation in obese subjects, while 63.4% (n=116) correlations were positive in lean controls (p=0.012). This result indicates that not only the number of interactions was depleted, but also the ratio between positive and negative interactions was imbalanced in obese subjects compared with lean controls. Depleted correlations in obese subjects were primarily driven by correlations between viral species from the Myoviridae, Siphoviridae and unclassified family, and bacterial species from the phylum Firmicutes (FIG. 20). In particular, Bacillus phage showed a strong positive correlation with its bacterial host (Bacillus cereus) in lean controls. Bacillus cereus strains were reported as probiotics for food animals to suppress Salmonella and Campylobacter in the gut²⁵. However, this correlation was significantly reduced in obese subjects. In the lean controls, other bacterial species belonging to Firmicutes showed robust positive interaction with Bacillus phage, as well as strong negative interactions between bacterial species belonging to Bacteroidetes and the phage species Bacillus phage.

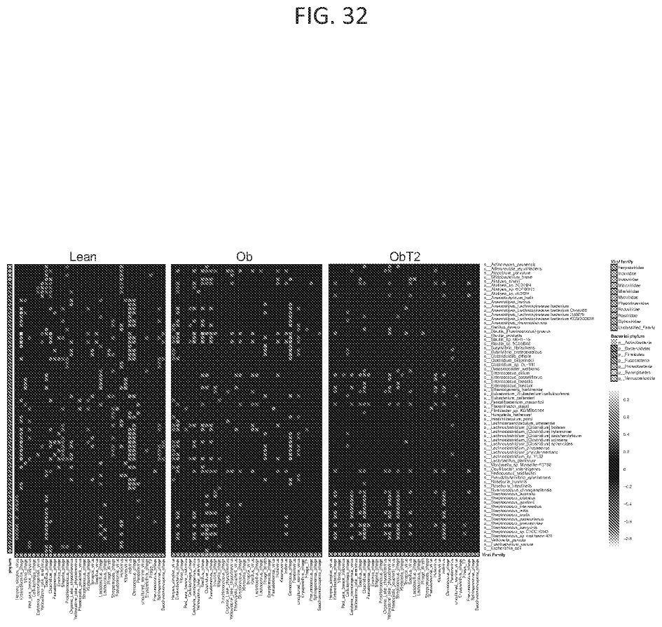

To explore the effect of T2DM on viral-bacterial interactions, inter-kingdom correlations difference between ObT2, Ob and lean controls was explored. There was decreased number of inter-kingdom correlations in Ob compared with lean controls (250 vs 317; p=0.003; FIG. 32). In particular, the number of inter-kingdom correlations was reduced more significantly in ObT2 (176 vs 317; p<0.001; FIG. 32), including positive correlations between viruses and bacteria from the phylum Firmicutes. Altogether, these results indicate that a strong viral-bacterial inter-kingdom interaction may contribute to maintaining a healthy ecological network of the gut microbiota in lean subjects which was substantially lost in obesity and T2DM.

Bacteriophages, which are the predominant members in the gut virome, are widely reported to be associated with bacterial microbiome ecology and host health³⁰. Data obtained in this study indicate a complex ecological network between gut virome and bacteriome in lean controls, while the correlations were markedly weakened in obesity, especially in obese subject who also had T2DM. Among the inter-kingdom correlations, several virus-bacteria correlations were seen beyond the common phage-host relationship suggesting a complex ecological system between gut virome and bacteriome in shaping a healthy gut microbiota. Lactic acid and Short-Chain Fatty Acids (SCFA) producing bacteria including Bifidobacterium breve, Blautia spp. and species under Lachnoclostridium, showed a strong positive interaction with gut viruses in lean controls. SCFA producing bacteria are known to exert a beneficial effect on metabolic diseases^{31,32}. Correlations between these probiotic bacteria and gut viruses highlight the potential role of gut virome in shaping a healthy gut microbiota.

As such, restoration of inter-kingdom interactions by FMT is useful for reduction of the risk, development, and progress of obesity/excess body weight and control of type 2 diabetes. Furthermore, administration of Bacillus phage, or Bacillus cereus or both to subjects with obesity or type 2 diabetes is useful for reduction of body weight and control of type 2 diabetes, whereas administration of Bifidobacterium breve, Blautia spp. or species under Lachnoclostridium to subjects with obesity or type 2 diabetes is useful for reduction of body weight and control of type 2 diabetes by boosting inter-kingdom interactions.

All patents, patent applications, and other publications, including GenBank Accession Numbers and the like, cited in this application are incorporated by reference in the entirety for all purposes.

### REFERENCES

1. Yach D, Stuckler D, Brownell K D. Epidemiologic and economic consequences of the global epidemics of obesity and diabetes. Nat Med 2006; 12:62-66.
2. Jiang Y, Xu Y, Bi Y, et al. Prevalence and trends in overweight and obesity among Chinese adults in 2004-10: data from three nationwide surveys in China. The Lancet 2015; 386:S77.
3. Xu H, Cupples L A, Stokes A, et al. Association of Obesity With Mortality Over 24 Years of Weight History: Findings From the Framingham Heart Study. JAMA Netw Open 2018; 1:e184587—e184587.
4. Anon. Obesity and overweight. Available at: https://www.who.int/news-room/fact-sheets/detail/obesity-and-overweight [Accessed May 10, 2019].
5. WHO. Diabetes. Available at: https://www.who.int/westernpacific/health-topics/diabetes [Accessed Feb. 17, 2020].
6. Guo F, Garvey W T. Cardiometabolic Disease Staging Predicts Effectiveness of Weight-Loss Therapy to Prevent Type 2 Diabetes: Pooled Results From Phase III Clinical Trials Assessing Phentermine/Topiramate Extended Release. Diabetes Care 2017; 40:856-862.
7. Lazar M A. How Obesity Causes Diabetes: Not a Tall Tale. Science 2005; 307:373-375.
8. Qin J, Li Y, Cal Z, et al. A metagenome-wide association study of gut microbiota in type 2 diabetes. Nature 2012; 490:55-60.
9. Liu R, Hong J, Xu X, et al. Gut microbiome and serum metabolome alterations in obesity and after weight-loss intervention. Nat Med 2017; 23:859-868.
10. Hartstra A V, Bouter K E C, Bäckhed F, et al. Insights Into the Role of the Microbiome in Obesity and Type 2 Diabetes. Diabetes Care 2015; 38:159-165.
11. Hossain P, Kawar B, El Nahas M. Obesity and Diabetes in the Developing World—A Growing Challenge. N Engl J Med 2007; 356:213-215.
12. Thingholm L B, Rühlemann MC, Koch M, et al. Obese Individuals with and without Type 2 Diabetes Show Different Gut Microbial Functional Capacity and Composition. Cell Host Microbe 2019; 0. Available at: https://www.cell.com/cell-host-microbe/abstract/S1931-3128(19)30348-8 [Accessed Aug. 7, 2019].
13. Ogilvie L A, Jones B V. The human gut virome: a multifaceted majority. Front Microbiol 2015; 6. Available at: https://www.frontiersin.org/articles/10.3389/fmicb.2015.00918/full [Accessed Apr. 24, 2018].
14. Reyes A, Haynes M, Hanson N, et al. Viruses in the faecal microbiota of monozygotic twins and their mothers. Nature 2010; 466:334-338.
15. Moreno-Gallego J L, Chou S-P, Rienzi S C D, et al. Virome Diversity Correlates with Intestinal Microbiome Diversity in Adult Monozygotic Twins. Cell Host Microbe 2019; 25:261-272.e5.
16. Shkoporov A N, Hill C. Bacteriophages of the Human Gut: The “Known Unknown” of the Microbiome. Cell Host Microbe 2019; 25:195-209.
17. Norman J M, Handley S A, Baldridge M T, et al. Disease-Specific Alterations in the Enteric Virome in Inflammatory Bowel Disease. Cell 2015; 160:447-460.
18. Zuo T, Lu X-J, Zhang Y, et al. Gut mucosal virome alterations in ulcerative colitis. Gut 2019:gutjnl-2018-318131.
19. Zhao G, Vatanen T, Droit L, et al. Intestinal virome changes precede autoimmunity in type I diabetes-susceptible children. Proc Natl Acad Sci 2017:201706359.
20. Ma Y, You X, Mai G, et al. A human gut phage catalog correlates the gut phageome with type 2 diabetes. Microbiome 2018; 6:24.
21. Rasmussen T S, Mentzel C M J, Kot W, et al. Faecal virome transplantation decreases symptoms of type 2 diabetes and obesity in a murine model. Gut 2020:gutjnl-2019-320005.
22. Zuo T, Wong S H, Lam K, et al. Bacteriophage transfer during faecal microbiota transplantation in Clostridium difficile infection is associated with treatment outcome. Gut 2017:gutjnl-2017-313952.
23. Hannigan G D, Meisel J S, Tyldsley A S, et al. The Human Skin Double-Stranded DNA Virome: Topographical and Temporal Diversity, Genetic Enrichment, and Dynamic Associations with the Host Microbiome. mBio 2015; 6:e01578-15.
24. Draper L A, Ryan F J, Dalmasso M, et al. Autochthonous faecal virome transplantation (FVT) reshapes the murine microbiome after antibiotic perturbation. Microbiology; 2019. Available at: http://biorxiv.org/lookup/doi/10.1101/591099 [Accessed Nov. 26, 2019].
25. Cutting S M. Bacillus probiotics. Food Microbiol 2011; 28:214-220.
26. Koutnikova H, Genser B, Monteiro-Sepulveda M, et al. Impact of bacterial probiotics on obesity, diabetes and non-alcoholic fatty liver disease related variables: a systematic review and meta-analysis of randomised controlled trials. BMJ Open 2019; 9:e017995.
27. Foligné B, Dewulf J, Breton J, et al. Probiotic properties of non-conventional lactic acid bacteria: Immunomodulation by Oenococcus oeni. Int J Food Microbiol 2010; 140:136-145.
28. Sakaguchi Y, Hayashi T, Kurokawa K, et al. The genome sequence of Clostridium botulinum type C neurotoxin-converting phage and the molecular mechanisms of unstable lysogeny. Proc Natl Acad Sci 2005; 102:17472-17477.
29. Bustamante F, Brunaldi V O, Bernardo W M, et al. Obesity Treatment with Botulinum Toxin-A Is Not Effective: a Systematic Review and Meta-Analysis. Obes Surg 2017; 27:2716-2723.
30. Mirzaei M K, Maurice C F. Ménage à trois in the human gut: interactions between host, bacteria and phages. Nat Rev Microbiol 2017; 15:397-408.
31. MINAMI J, IWABUCHI N, TANAKA M, et al. Effects of Bifidobacterium breve B-3 on body fat reductions in pre-obese adults: a randomized, double-blind, placebo-controlled trial. Biosci Microbiota Food Health 2018; 37:67-75.
32. Kobyliak N, Conte C, Cammarota G, et al. Probiotics in prevention and treatment of obesity: a critical view. Nutr Metab 2016; 13. Available at: https://www.ncbi.nlm.nih.gov/pmc/articles/PMC4761174/[Accessed May 26, 2020].
33. Parras-Moltó M, Rodríguez-Galet A, Suárez-Rodríguez P, et al. Evaluation of bias induced by viral enrichment and random amplification protocols in metagenomic surveys of saliva DNA viruses. Microbiome 2018; 6:119.
34. Edwards R A, Rohwer F. Viral metagenomics. Nat Rev Microbiol 2005; 3:504-510.
35. Kim Min-Soo, Bae Jin-Woo. Spatial disturbances in altered mucosal and luminal gut viromes of diet-induced obese mice. Environ Microbiol 2016; 18:1498-1510.
36. Minot S, Sinha R, Chen J, et al. The human gut virome: Inter-individual variation and dynamic response to diet. Genome Res 2011; 21:1616-1625.
37. Vich Vila A, Collij V, Sanna S, et al. Impact of commonly used drugs on the composition and metabolic function of the gut microbiota. Nat Commun 2020; 11:362.

1. A method for reducing the risk of a metabolic disease or treating a metabolic disease in a subject, comprising administering to the subject a composition comprising an effective amount of one or more of the microbial species selected from the group consisting of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and viruses in Table 9.

2. The method of claim 1, wherein the metabolic disease is obesity, pre-diabetes, or type-2 diabetes.

3. The method of claim 1, wherein the administering step comprises oral administration or delivery to the small intestine, ileum, or large intestine of the subject.

4. The method of claim 1, wherein the administering step comprises fecal microbiota transplantation (FMT).

5. The method of claim 4, wherein the FMT comprises administration to the subject a composition comprising processed donor fecal material.

6. The method of claim 1, wherein the composition comprises no detectable amount of any virus in Table 7 or 8.

7. The method of claim 6, wherein the composition comprises no detectable amount of Ugandan cassava brown streak virus.

8. The method of claim 1, wherein high-density lipoprotein cholesterol (HDL-C) level is increased, low-density lipoprotein cholesterol (LDL-C) level is decreased, and/or blood glucose level is decreased in the subject.

9. The method of claim 1, wherein bodyweight is reduced in the subject.

10. A kit for reducing the risk of a metabolic disease or treating a metabolic disease, comprising: a first container containing a first a composition comprising an effective amount of one microbial species selected from the group consisting of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and viruses in Table 9, and a second container containing a second composition comprising an effective amount of another microbial species selected from the group consisting of Diachasmimorpha longicaudata entomopoxvirus, Megavirus, Oenococcus phage, Saudi moumouvirus, Clostridium botulinum C phage, Emiliania huxleyi virus, Lausannevirus, Gokushovirus, Bacillus phage, Escherichia phage, Streptococcus phage, Microvirus, Candida dubliniesis, Bacillus cereus, Bifidobacterium breve, Blautia spp., species under Lachnoclostridium, and viruses in Table 9.

11. The kit of claim 10, wherein the first and/or second composition comprises processed donor fecal material for FMT.

12. The kit of claim 10, wherein the first and/or second composition is formulated for oral administration.

13. The kit of claim 10, further comprising a third container containing a third composition comprising an effective amount of an antiviral agent inhibiting the viruses in Tables 7 and 8.

14. The kit of claim 13, wherein the antiviral agent inhibits Ugandan cassava brown streak virus.

15. A method for assessing risk of developing a metabolic disease among two subjects, comprising:
(1) determining, in a stool sample from a first subject, the level or relative abundance of one or more of the viral species selected from the group consisting of Bacteroides phage, Pectobacterium phage, Achromobacter phage, Azobacteroides phage, crAssphage, and the viruses in Tables 7 and 8;
(2) detecting the level or relative abundance from step (1) being higher than the level or relative abundance of the same virial species in a stool sample from a second subject; and
(3) determining the first subject as having a higher risk of developing a metabolic disease than the second subject.

16. The method of claim 15, wherein the one or more viral species comprise Ugandan cassava brown streak virus.

17. A kit for assessing developing a metabolic disease in a subject, comprising reagents for detecting one or more of the virial species selected from the group consisting of Bacteroides phage, Pectobacterium phage, Achromobacter phage, Azobacteroides phage, crAssphage, and the viruses in Tables 7 and 8.

18. The kit of claim 39, wherein the reagents comprise a set of oligonucleotide primers for amplification of a polynucleotide sequence from any one of Bacteroides phage, Pectobacterium phage, Achromobacter phage, Azobacteroides phage, and crAssphage, or the virial species in Tables 7 and 8.

19. The kit of claim 18, wherein the one or more viral species comprise Ugandan cassava brown streak virus.

20. The kit of claim 19, wherein the amplification is PCR, preferably quantitative PCR (qPCR).

21. A method for determining risk for obesity and/or type 2 diabetes risk in an obese test subject, comprising:
(a) quantitatively determining the relative abundance of viral species selected from Table 10, Table 13, or Table 16 in a stool sample taken from the test subject;
(b) quantitatively determining the relative abundance of viral species selected from Table 10, Table 13, or Table 16 in a stool sample taken from a reference cohort comprising obese subjects, obese with type 2 diabetes subjects, and lean controls;
(c) generating decision trees by random forest model using data obtained from (b);
(d) running the relative abundance obtained from (a) down the decision trees from (b) to generate a risk score; and
(e) determining the test subject with a score greater than 0.5 as having an increased risk for obesity and/or type 2 diabetes, and determining the test subject with a score no greater than 0.5 as having no increased risk for obesity and/or type 2 diabetes.

22. A method for determining obesity risk in a test subject, comprising:
(1) obtaining from a cohort of obese subjects and lean controls a set of training data by determine the age of subjects and relative abundance of viral species Staphylococcus virus, Phormidium phage, and Costridium virus in stool samples;
(2) determining the relative abundance of the viral species in a stool sample taken from the test subject whose risk of obesity is to be determined;
(3) comparing the relative abundance of the viral species from step (2) with the training data using random forest model;
(4) generating decision trees by random forest from the training data and running the relative abundance from step (2) down the decision trees to generated a risk score; and
(5) determining the test subject with a risk score greater than 0.5 as at increased risk for obesity and determining the test subject with a risk score no greater than 0.5 as at no increased risk for obesity.

23. The method of claim 22, wherein the viral species further comprise Hepatitis C virus and/or Catovirus.

24. A method for determining risk of obesity with type 2 diabetes in a test subject, comprising:
(1) obtaining from a cohort of obese with type 2 diabetes subjects and lean controls a set of training data by determine the age of subjects and relative abundance of viral species Achromobacter phage, Oenococcus phage, and Geobacillus phage in stool samples;
(2) determining the relative abundance of the viral species in a stool sample taken from the test subject whose risk of obesity with type 2 diabetes is to be determined;
(3) comparing the relative abundance of the viral species from step (2) with the training data using random forest model;
(4) generating decision trees by random forest from the training data and running the relative abundance from step (2) down the decision trees to generated a risk score; and
(5) determining the test subject with a risk score greater than 0.5 as at increased risk for obesity with type 2 diabetes and determining the test subject with a risk score no greater than 0.5 as at no increased risk for obesity with type 2 diabetes.

25. The method of claim 24, wherein the viral species further comprise one or more of Mycoplasma phage, Klosneuvirus, and Fowl aviadenovirus.

26. A method for determining type 2 diabetes risk in an obese test subject, comprising:
(1) obtaining from a cohort of obese with type 2 diabetes subjects and obese controls a set of training data by determine the age of subjects and relative abundance of viral species Oenococcus phage and Bradyrhizobium phage in stool samples;
(2) determining the relative abundance of the viral species in a stool sample taken from the test subject whose type 2 diabetes risk is to be determined;
(3) comparing the relative abundance of the viral species from step (2) with the training data using random forest model;
(4) generating decision trees by random forest from the training data and running the relative abundance from step (2) down the decision trees to generated a risk score; and
(5) determining the test subject with a risk score greater than 0.5 as at increased risk for type 2 diabetes and determining the test subject with a risk score no greater than 0.5 as at no increased risk for type 2 diabetes.

27. The method of claim 26, wherein the viral species further comprise one or more of Phormidium phage, Heliothis zea nudivirus, and Achromobacter phage.
